(19) United States
(12) Patent Application Publication
(10) Pub. No.: US 2026/0284014 A1
(43) Pub. Date: Sep. 24, 2026

(54) 3,5-DIAMINOTRIAZOLE DERIVATIVES AND USES THEREOF FOR TREATING OCULAR DISEASES

(71) Applicants: CENTRE NATIONAL DE LA RECHERCHE SCIENTIFIQUE, PARIS (FR); INSTITUT NATIONAL DE LA SANTÉ ET DE LA RECHERCHE MÉDICALE, PARIS (FR); UNIVERSITE COTE D'AZUR, NICE (FR)

(72) Inventors: RACHID BENHIDA, NICE (FR); MAEVA DUFIES, NICE (FR); MARIE FABRE, VALBONNE (FR); LETICIA GONCALVES-PIRES, NICE (FR); JULIE LE DU, NICE (FR); GILLES PAGES, MONACO (MC); OLIVIA RASTOIN, NICE (FR); CYRIL RONCO, NICE (FR)

(21) Appl. No.: 19/476,244

(22) PCT Filed: Apr. 18, 2024
(86) PCT No.: PCT/EP2024/060593

§ 371 Date: Oct. 17, 2025

(30) Foreign Application Priority Data
Apr. 18, 2023 (EP) 23305589.6

(51) Int. Cl. A61K 31/4196; A61K 31/4439; A61P 27/02; C07D 249/14; C07D 401/12

(52) U.S. Cl. CPC A61K 31/4196; A61K 31/4439; A61P 27/02; C07D 249/14; C07D 401/12

(57) ABSTRACT

The present invention relates to 3,5-diaminotriazole derivatives of formula (I) and pharmaceutical or veterinary compositions comprising such derivatives. It further relates to the compounds of formula (I) for use for treating an ocular disease, preferably diabetic retinopathy, macular degeneration, neovascular glaucoma, macular oedema or Stargardt's disease.

### FIELD OF THE INVENTION

The present invention relates to the field of medicine, in particular to 3,5-diaminotriazole derivatives, and uses thereof in the treatment of ocular diseases.

### BACKGROUND OF THE INVENTION

Angiogenesis is a physiological process involving the formation of new capillary blood vessels from pre-existing micro-vessels. There are a large number of diseases characterized by an exacerbated or impaired angiogenesis and increased inflammation such as eye diseases. Eye diseases (also called herein ocular disease) associated with angiogenesis include, without limitation, corneal graft angiogenesis, neovascular glaucoma, diabetic retinopathy, corneal diseases induced by new blood vessels, macular degeneration, pterygium, retinal degeneration, retrolental fibroplasia, granular conjunctivitis, Stargardt's disease, and the like. The overdevelopment of the blood vessels can often lead to severe damage for the patient. Macular degeneration, for example, impairs central vision and, in acute cases, can lead to blindness. Also, 30% of patients suffering from neovascular glaucoma are forced to undergo enucleation to relieve the pain.

In this context, there is an urgent need for the development of new therapeutic approaches to prevent and/or treat highly vascularized and inflammatory eye diseases. In 2012, WO 2012/027289 disclosed aminopyridine- and aminopyrimidine carboxamide derivatives targeting CXCR2 receptors, that may be suitable for the treatment of inflammatory and neoplastic disorders such as angiogenic eye diseases, ocular inflammation, diabetic retinopathy, macular degeneration and corneal neovascularization. A decade later, Wang et al. (Redox Biol., 2022, 56, 102438) reported that selective blockade of CXCL1-CXCL2 activation could be a potential treatment for hypertensive retinopathy. Monickaraj et al. (Investigative Ophthalmology & Visual Science, 2021, 62, 3032) reported on the role of the chemokine CXCL1 in altering the blood-retinal barrier in diabetic retinopathy, and thus as a potential new therapeutic target.

In previous studies, the inventors reported the use of urea derivatives as CXCR1 and CXCR2 receptor antagonists for the treatment of macular degeneration (WO 2020/079184). They also reported in WO 2022/219123 that these urea derivatives are effective in treating uveal melanoma by inhibiting the production of ROS, which is also known to modulate angiogenesis and inflammation in neovascular glaucoma (Masuda et al.: Oxid. Med. Cell Longev., Volume 2017; Nita et al.: Oxid. Med. Cell Longev., Volume 2016).

There are a variety of targets (ROS species, chemokines receptors . . . ) and others remain to be investigated to develop new therapeutic approaches for the treatment of ocular diseases. Therefore, there is a need for the development of new drugs or new compounds that have a therapeutic effect against eye diseases (or ocular diseases) and at the same time are being safe for the patient.

### SUMMARY OF THE INVENTION

In this context, the inventors have synthesized and provided novel 3,5-diaminotriazole derivatives of formula (I). They demonstrated a therapeutic interest, particularly for the treatment of ocular diseases, especially those associated with an exacerbated angiogenesis. More specifically, the inventors have shown that the compounds of formula (I) according to the invention have inhibitory properties, such as an inhibitory effect on therapeutic targets associated with ocular diseases. The inventors have further shown that the compounds of formula (I) according to the invention were able to inhibit ocular (retinal) neo-angiogenesis in the zebrafish model.

The present invention therefore relates to a compound of formula (I) or a pharmaceutically acceptable salt thereof:

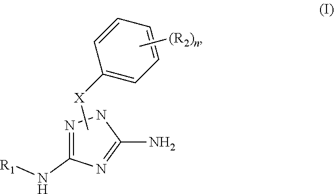

in which:
   X is —(CH₂)—, —(CH₂)—CO—, or —(CH₂)—(CH₂)—;
   R₁ is an aryl or a heteroaryl group, said aryl or heteroaryl groups are optionally substituted by at least one radical selected in the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro;
   n is an integer number comprised between 0 and 5; and
   each R₂ is independently a radical selected in the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro;
with the proviso that said compound is not a compound selected in a group consisting of:
   1-benzyl-N-phenyl-1H-1,2,4-triazole-3,5-diamine; and
   1-phenethyl-N³-phenyl-1H-1,2,4-triazole-3,5-diamine.

Particularly, n is an integer number comprised between 1 and 5.

In a particular embodiment, the compound of the invention is of the following formula (IA) or (IB):

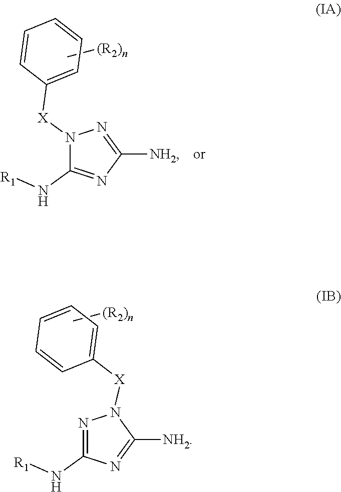

Preferably, the compounds of the invention of formulae (I), (IA), and (IB) are such that X is —(CH₂)—.

In a particular embodiment, the compounds of the invention of formulae (I), (IA), and (IB) are such that R₁ is a phenyl or a pyridinyl group, preferably a phenyl, said phenyl or pyridinyl groups are optionally substituted by at least one radical selected in the group consisting of a (C₁-C₆)alkyloxy, preferably a methoxy, a (C₁-C₆)alkyl, preferably a methyl, a halogen, preferably a chlorine or a bromine, and a —CO₂R₃ with R₃ being a (C₁-C₆)alkyl, preferably a methyl.

In a further particular, the compounds of the invention of formulae (I), (IA), and (IB) are such that n is an integer comprised between 0 and 3, preferably n is 0, 1, or 2, more preferably 1.

In a further particular, the compounds of the invention of formulae (I), (IA), and (IB) are such that n is an integer comprised between 1 and 3, preferably n is 1 or 2, more preferably 1.

More particularly, each R₂ is independently a radical selected in the group consisting of a (C₁-C₆)alkyl optionally substituted by at least one halogen, preferably a methyl, a trifluoromethyl, or an isopropyl, a (C₁-C₆)alkyloxy, preferably a methoxy, a halogen, preferably a fluorine, a bromine, or a chlorine, a nitro, and a nitrile. In a preferred embodiment, each R₂ is independently a radical selected in the group consisting of a an isopropyl, and a bromine, preferably a bromine.

In a preferred embodiment, a compound of the invention is a compound of formula (I), (IA), or (IB) in which:
X is —(CH₂)—;
R₁ is a phenyl or a pyridinyl group, preferably a phenyl, said phenyl or pyridinyl groups are optionally substituted by at least one radical selected in the group consisting of a methyl, a chlorine, a bromine, and a —CO₂R₃ with R₃ being a methyl;
n is 1; and
R₂ is a radical selected in the group consisting of an isopropyl, and a bromine.

In a more preferred embodiment, the compound of formulae (I), (IA), and (IB) is selected in the group consisting of:
MCK248: 1-benzyl-N⁵-phenyl-1H-1,2,4-triazole-3,5-diamine;
MCK250: 1-(2-methylbenzyl)-N³-phenyl-1H-1,2,4-triazole-3,5-diamine;
MCK253: 1-benzyl-N³-(pyridin-2-yl)-1H-1,2,4-triazole-3,5-diamine;
MCK254: 1-benzyl-N³-(pyridin-3-yl)-1H-1,2,4-triazole-3,5-diamine;
MCK257: 1-(2,6-difluorobenzyl)-N⁵-phenyl-1H-1,2,4-triazole-3,5-diamine;
MCK259: 1-(2,6-difluorobenzyl)-N⁵-(pyridin-2-yl)-1H-1,2,4-triazole-3,5-diamine;
MCK260: 1-(3-methoxybenzyl)-N⁵-phenyl-1H-1,2,4-triazole-3,5-diamine;
MCK261: 1-(3-methoxybenzyl)-N³-phenyl-1H-1,2,4-triazole-3,5-diamine;
MCK262: 1-(4-isopropylbenzyl)-N⁵-phenyl-1H-1,2,4-triazole-3,5-diamine;
MCK263: 1-(4-isopropylbenzyl)-N³-phenyl-1H-1,2,4-triazole-3,5-diamine;
MCK264: 1-(3-bromobenzyl)-N³-phenyl-1H-1,2,4-triazole-3,5-diamine;
MCK268: 1-(2,6-dichlorobenzyl)-N³-phenyl-1H-1,2,4-triazole-3,5-diamine;
MCK269: 1-(2-methylbenzyl)-N³-(pyridin-2-yl)-1H-1,2,4-triazole-3,5-diamine;
MCK271: 1-(4-methylbenzyl)-N³-phenyl-1H-1,2,4-triazole-3,5-diamine;
MCK272: 1-(4-chlorobenzyl)-N³-phenyl-1H-1,2,4-triazole-3,5-diamine;
MCK273: N³-phenyl-1-(3-(trifluoromethyl)benzyl)-1H-1,2,4-triazole-3,5-diamine;
MCK274: 1-(3,5-difluorobenzyl)-N³-phenyl-1H-1,2,4-triazole-3,5-diamine;
MCK275: 1-(3-nitrobenzyl))-N³-phenyl-1H-1,2,4-triazole-3,5-diamine;
MCK276: 1-(3-bromobenzyl)-N³-(pyridin-2-yl)-1H-1,2,4-triazole-3,5-diamine;
MCK278: 1-(2,6-dichlorobenzyl)-N³-(pyridin-2-yl)-1H-1,2,4-triazole-3,5-diamine;
MCK279: 1-(4-methylbenzyl)-N³-(pyridin-2-yl)-1H-1,2,4-triazole-3,5-diamine;
MCK280: 1-(4-chlorobenzyl)-N³-(pyridin-2-yl)-1H-1,2,4-triazole-3,5-diamine;
MCK281: 4-((5-amino-3-(pyridin-2-ylamino)-1H-1,2,4-triazol-1-yl)methyl)benzonitrile;
MCK282: N³-(pyridin-2-yl)-1-(3-(trifluoromethyl)benzyl)-1H-1,2,4-triazole-3,5-diamine;
MCK283: 1-(3,5-difluorobenzyl)-N³-(pyridin-2-yl)-1H-1,2,4-triazole-3,5-diamine;
MCK284: 1-(2,4-difluorobenzyl)-N³-(pyridin-2-yl)-1H-1,2,4-triazole-3,5-diamine;
MCK288: 1-(4-nitrobenzyl))-N³-phenyl-1H-1,2,4-triazole-3,5-diamine;
MCK289: 1-(2-nitrobenzyl)-N³-(pyridin-2-yl)-1H-1,2,4-triazole-3,5-diamine;
MCK290: 1-(4-nitrobenzyl)-N³-(pyridin-2-yl)-1H-1,2,4-triazole-3,5-diamine;
MCK292: 1-(3-nitrobenzyl)-N³-(pyridin-2-yl)-1H-1,2,4-triazole-3,5-diamine;
MCK295: 2-(5-amino-3-(pyridin-2-ylamino)-1H-1,2,4-triazol-1-yl)-1-(3-nitrophenyl)ethan-1-one;
MCK297: 1-(2-nitrobenzyl)-N³-phenyl-1H-1,2,4-triazole-3,5-diamine;
MCK298: N³-phenyl-1-(3,4,5-trimethoxybenzyl)-1H-1,2,4-triazole-3,5-diamine;
MCK299: 2-(5-amino-3-(phenylamino)-1H-1,2,4-triazol-1-yl)-1-phenylethan-1-one;
MCK300: 1-phenethyl-N-phenyl-1H-1,2,4-triazole-3,5-diamine;
MCK338: N⁵-phenyl-1-(3-(trifluoromethyl)benzyl)-1H-1,2,4-triazole-3,5-diamine;
MCK339: 1-(3-bromobenzyl)-N⁵-phenyl-1H-1,2,4-triazole-3,5-diamine;
MCK340: 1-(3-bromobenzyl)-N³-(4-methoxyphenyl)-1H-1,2,4-triazole-3,5-diamine;
MCK341: 1-(3-bromobenzyl)-N³-(4-methylphenyl)-1H-1,2,4-triazole-3,5-diamine;
MCK342: 1-(3-bromobenzyl)-N³-(4-methylphenyl)-1H-1,2,4-triazole-3,5-diamine;
MCK343: 1-(3-bromobenzyl)-N⁵-(4-chlorophenyl)-1H-1,2,4-triazole-3,5-diamine;
MCK344: 1-(3-bromobenzyl)-N³-(4-chlorophenyl)-1H-1,2,4-triazole-3,5-diamine;
MCK345: 1-(3-bromobenzyl)-N⁵-(4-bromophenyl)-1H-1,2,4-triazole-3,5-diamine;
MCK346: 1-(3-bromobenzyl)-N³-(4-bromophenyl)-1H-1,2,4-triazole-3,5-diamine;
MCK347: methyl-4-((3-amino-1-(3-bromobenzyl)-1H-1,2,4-triazol-5-yl)amino)benzoate;
MCK348: methyl-4-((5-amino-1-(3-bromobenzyl)-1H-1,2,4-triazol-3-yl)amino)benzoate;
MCK349: 1-(3-bromobenzyl)-N³-(3,4-dichlorophenyl)-1H-1,2,4-triazole-3,5-diamine;
MCK350: N³-(3,4-dichlorophenyl)-1-(4-isopropylbenzyl)-1H-1,2,4-triazole-3,5-diamine;
MCK351: 1-(3-bromobenzyl)-N³-(pyridin-3-yl)-1H-1,2,4-triazole-3,5-diamine; and
MCK352: 1-(3-bromobenzyl)-N⁴-(pyridin-3-yl)-1H-1,2,4-triazole-3,5-diamine.

Another object of the invention is a compound according to the invention as defined herein for use as a drug or a medicament. Another object is a pharmaceutical or veterinary composition comprising a compound as defined herein, and a pharmaceutically acceptable excipient.

Another object is a compound or a pharmaceutical or veterinary composition according to the invention for use for treating an ocular disease. In a particular embodiment, the ocular disease is chosen among corneal graft angiogenesis, neovascular glaucoma, diabetic retinopathy, macular edema, corneal diseases induced by new blood vessels, macular degeneration, pterygium, retinal degeneration, retrolental fibroplasia, granular conjunctivitis, macular edema, or Stargardt's disease. In a preferred embodiment, the ocular disease is chosen among diabetic retinopathy, macular degeneration, neovascular glaucoma, macular edema, or Stargardt's disease.

In a particular embodiment, the pharmaceutical or veterinary composition for use according to the invention is administered by oral, parenteral, or topical route, preferably by topical route, more preferably by ocular topical route.

### LEGEND OF FIGURES

FIG. 1: MCK349 inhibits oxidative stress in ARPE and angiogenesis. A. ARPE-19 cells were treated with 1 μM MCK349 followed by 200 μM H₂O₂ for 2 h. ROS were analyzed by cytometry. B. ARPE-19 cells were treated with 2.5 μM MCK349 for 48 h and CXCL1 mRNA level was evaluated by qPCR. C. TIME cells were pre-treated with 2.5 μM MCK349 for 1 h then stimulated with 100 ng/ml CXCL8 for 15 min. The p-ERK levels were analyzed by immunoblotting. HSP90 served as loading control. D. Formation of vessels by TIME cells cultured on Matrigel and treated with 5 or 10 μM MCK349. E and F. TIME cells were incubated for 72 h with 100 ng/mL CXCL8 (E) or CXCL7 (F) and MCK349 (0.1, 0.2 or 0.5 μM). Cell proliferation is determined by cell number. Results are presented as the mean of three independent experiments SEM. For A and B, statistical significance (compared with the control condition) was determined using an unpaired Student's 1-test: ***P<0.001. For E and F, statistical significance (compared with the CXCL-8 or CXCL7 condition) was determined using one-way ANOVA with Bonferroni post hoc: ***P<0.001.

FIG. 2: MCK349 and MCK342 inhibit pro-inflammatory cytokines in human blood stimulated with LPS. Human whole blood will be incubated with MCK349 or MCK342 (5 or 10 μM) for 1 h prior to 10 ng/ml LPS stimulation for 18 h. IL1ß, IL6, INFγ, IL4, IL5 and IL10 cytokines were analyzed by multiplex ELISA. All analyzed cytokines were significantly increased in LPS condition in comparison with control condition. Statistical significance (compared with the LPS condition) was determined using one-way ANOVA with Bonferroni post hoc: *P<0.05; **P<0.01; ***P<0.001.

FIG. 3: MCK349 inhibits pro-inflammatory, pro-angiogenic and/or pro-fibrotic mRNA markers in diabetic retinopathy in vitro. ARPE-19 cells were cultured in hyperglycaemic conditions (high glucose) and were pre-treated with MCK349 (5 or 10 μM) for 24 h and then stimulated with cytokines (10 ng/ml IL1ß, 25 ng/ml VEGF and 25 ng/ml TNFα) for 24 h. IL1ß, VEGFC, TNFα, CXCL8, CXCL1, TGFß, MMP9, IL6 and IL33 mRNA were analyzed by qPCR. All analyzed mRNA markers were significantly increased in High glucose+cytokines condition in comparison with control condition. Statistical significance (compared with the High glucose+cytokines (CT) condition) was determined using one-way ANOVA with Bonferroni post hoc: *P<0.05; **P<0.01; ***P<0.001.

FIG. 4: MCK349 inhibits pro-inflammatory, pro-angiogenic and/or pro-fibrotic cytokines in diabetic retinopathy in vitro. ARPE-19 cells were cultured in hyperglycaemic conditions (high glucose) and were pre-treated with MCK349 (5, 10, or 25 μM) for 24 h and then stimulated with cytokines (10 ng/ml IL1ß, 25 ng/ml VEGF and 25 ng/ml TNFα) for 24 h. Cytokines (Il1ß, IFNγ, TNFα, CXCL8, IL4, IL2, IL13, IL10, IL5, IL12p70, IL6 and IL33) were analyzed by multiplex ELISA on the supernatant. All analyzed cytokines were significantly increased in High glucose+cytokines condition in comparison with control condition. Statistical significance (compared with the High glucose+cytokines (CT) condition) was determined using one-way ANOVA with Bonferroni post hoc: *P<0.05; **P<0.01; ***P<0.001.

FIG. 5: MCK263, MC342, MCK344 and MCK349 inhibit ocular (retinal) neo-angiogenesis in zebrafish. A to E. The transgenic zebrafish (fli: EGFP, blood vessels marked in GFP) was placed for 72 h in normoxia (Nx) or hypoxia 5% oxygen (Hx), in the presence of MCK263 (B), MC342 (C), MCK344 (D) or MCK349 (E)(0.25, 0.5, or 1 μM). Neo-angiogenesis in the retina was analyzed by confocal microscopy. A. Representative image is shown. On the image, the GFP blood vessels appear in white. B to E. Quantification of the area of blood vessels in the retina. Statistical significance (compared with the Hx condition) was determined using one-way ANOVA with Bonferroni post hoc: ***P<0.001.

FIG. 6: Inhibition of CXCL8 inducing ERK activation TIME cells were pre-treated with vehicle (CT) or 2.5 μM compound MCK295, MCK299 or MCK300 for 1 h then stimulated with 100 ng/ml CXCL8 for 15 min. p-ERK levels were analyzed by immunoblotting. HSP90 served as loading control.

FIG. 7: Inhibition of ROS activation by H₂O₂ ARPE-19 cells were treated with vehicle (CT) or 5 or 1 μM MCK248 or MCK249; or with 1 μM MCK263 or MCK264 followed by 200 μM H₂O₂ for 2 h. ROS were analyzed by cytometry.

FIG. 8: Inhibition of CXCL1 mRNA ARPE-19 cells were treated with vehicle (CT) or 2.5 μM MCK301 or MCK340 for 48 h and CXCL1 mRNA level was evaluated by qPCR.

FIG. 9: MCK349 inhibits choroidal neovascularization (CNV) and vascular leakage in mice. Choroidal neovascularization (CNV) in mice was induced by laser photocoagulation of retina. Vehicle (CT) and MCK349 (52 μg/ml) were delivered by binocular intravitreal injection (2 μl) four times in total. Once on Day 1 (right after modeling), Day 4, Day 7 and Day 11.

A. Grade 2b lesions (bright hyperfluorescence and late leakage beyond treated areas) were quantified by Fundus Fluorescein Angiography (FFA) at Day 9 and 15.

B. CNV volume of each lesion were measured at Day 9 and 15 by Optical Coherence Tomography (OCT).

C. Fundus Fluorescein Angiography (FFA) at Day 15 representative image are shown.

### DETAILED DESCRIPTION OF THE INVENTION

According to the present invention, the terms below have the following meanings:

The terms mentioned herein with prefixes such as for example C₁-C₃ or C₁-C₆ can also be used with lower numbers of carbon atoms such as C₁-C₂, or C₁-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, tert-butyl, pentyl or hexyl. In a preferred embodiment, the “alkyl” is a methyl, an ethyl, a propyl, an isopropyl, or a tert-butyl.

The term “alkyloxy” or “alkoxy” corresponds to the alkyl group as above defined bonded to the molecule by an —O— (ether) bond. (C₁-C₃)alkyloxy includes methoxy, ethoxy, propyloxy, and isopropyloxy. (C₁-C₆)alkyloxy includes methoxy, ethoxy, propyloxy, isopropyloxy, butyloxy, isobutyloxy, tert-butyloxy, pentyloxy and hexyloxy.

The term “halogen” corresponds to a fluorine, a chlorine, a bromine, or an iodine atom.

The term “aryl” corresponds to a mono- or bi- or tri- or tetra-yclic aromatic hydrocarbons having from 6 to 24 carbon atoms. For instance, the term “aryl” includes phenyl, naphtalenyl, anthracenyl, or pyrenyl. In a preferred embodiment, the aryl is a phenyl.

The term “heteroaryl” as used herein corresponds to an aromatic, mono- or poly-cyclic group comprising between 5 and 24 atoms and comprising at least one heteroatom such as nitrogen, oxygen or sulphur atom. As used herein, the term “heteroaryl” further includes the “fused arylheterocycloalkyl” and “fused heteroarylcycloalkyl”. The terms “fused arylheterocycloalkyl” and “fused heteroarylcycloalkyl” correspond to a bicyclic group in which an aryl as above defined or a heteroaryl is respectively bounded to the heterocycloalkyl or the cycloalkyl as above defined by at least two carbons. In other terms, the aryl or the heteroaryl shares a carbon bond with the heterocycloalkyl or the cycloalkyl. Examples of such mono- and poly-cyclic heteroaryl group, fused arylheterocycloalkyl and fused arylcycloalkyl may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, p-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolyl, benzoxazolinyl, benzoxazinyl, benzothienyl, benzothiazolyl, benzodiazepinyl, benzazepinyl, benzoxazepinyl, isatinyl, dihydropyridyl, pyrimidinyl, s-triazinyl, oxazolyl, or thiofuranyl. In a preferred embodiment, the heteroaryl is a pyridinyl.

The expression “substituted by at least one radical” means that the group or radical is substituted by one or several radicals of the list. The expression “optionally substituted” means that the group or radical is not substituted (i.e., unsubstituted) or substituted by one or several radicals of the list.

As used herein, the term “pharmaceutically acceptable salt” includes inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002.

### Compounds

The present invention relates to a compound of formula (I:

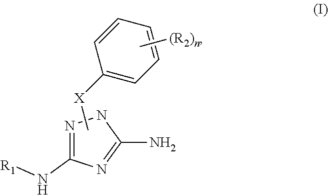

wherein
   X is —(CH₂)—, —(CH₂)—CO—, or —(CH₂)—(CH₂)—;
   R₁ is an aryl or a heteroaryl group, said aryl or heteroaryl groups are optionally substituted by at least one radical selected in the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro;
   n is an integer number comprised between 0 and 5;
   each R₂ is independently a radical selected in the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro;
or a pharmaceutically acceptable salt thereof.

In an embodiment, the present invention relates to a compound of formula (I):

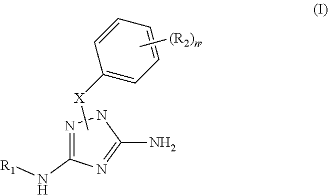

wherein:
   X is —(CH₂)—, —(CH₂)—CO—, or —(CH₂)—(CH₂)—;
   R₁ is an aryl or a heteroaryl group, said aryl or heteroaryl groups are optionally substituted by at least one radical selected in the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro;
   n is an integer number comprised between 0 and 5;
each R₂ is independently a radical selected in the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro;
with the proviso that said compound is not a compound selected in a group consisting of:
   1-benzyl-N³-phenyl-1H-1,2,4-triazole-3,5-diamine; and
   1-phenethyl-N³-phenyl-1H-1,2,4-triazole-3,5-diamine.
or a pharmaceutically acceptable salt thereof.

According to this embodiment, 1-benzyl-N³-phenyl-1H-1,2,4-triazole-3,5-diamine and 1-phenethyl-N³-phenyl-1H-1,2,4-triazole-3,5-diamine having the following formulae,

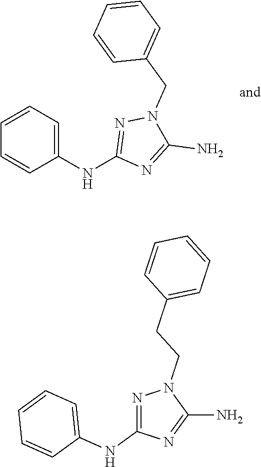 [SMILES: Nc1nc(Nc2ccccc2)nn1Cc1ccccc1 ; Nc1nc(Nc2ccccc2)nn1CCc1ccccc1]

are excluded.

In a particular embodiment, the compounds of the invention are of the following formula (IA):

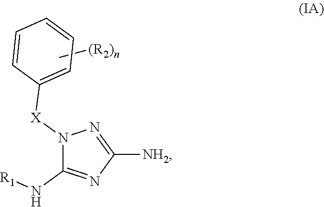

with X, R₁, n, and R₂ are such as defined herein.

In a further particular embodiment, the compounds of the invention are of the following formula (IB):

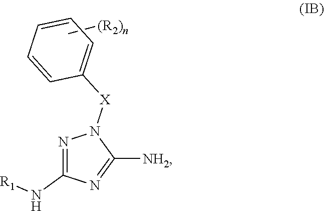

with X, R₁, n, and R₂ are such as defined herein.

According to the invention, X is —(CH₂)—, —(CH₂)—CO—, or —(CH₂)—(CH₂)—, preferably —(CH₂)—.

According to the invention, R₁ is an aryl or a heteroaryl group, optionally substituted by at least one radical selected in the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro.

In a particular embodiment, R₁ is an aryl or a heteroaryl group, preferably a phenyl or a pyridinyl group, optionally substituted by at least one radical selected in the group consisting of a (C₁-C₆)alkyl optionally substituted by at least one halogen, a (C₁-C₆)alkyloxy optionally substituted by at least one halogen, a halogen, and a —CO₂R₃ with R₃ being a hydrogen or a (C₁-C₆)alkyl.

In a more particular embodiment, R₁ is a phenyl or a pyridinyl group, preferably a phenyl group, said phenyl or pyridinyl groups are optionally substituted by at least one radical selected in the group consisting of a (C₁-C₆)alkyloxy, a (C₁-C₆)alkyl, a halogen, and a —CO₂R₃ with R₃ being a (C₁-C₆)alkyl. In an even more particular embodiment, R₁ is a phenyl or a pyridinyl group, preferably a phenyl group, said phenyl or pyridinyl groups are optionally substituted by at least one radical selected in the group consisting of a methoxy, a methyl, a halogen, preferably a chlorine or a bromine, and a —CO₂R₃ with R₃ being a methyl.

In a preferred embodiment, R₁ is a phenyl group optionally substituted by at least one radical selected in the group consisting of a methoxy, a methyl, a chlorine, a bromine, and a —CO₂R₃ with R₃ being a methyl.

In a further preferred embodiment, R₁ is a pyridinyl group.

According to the invention, n is an integer comprised between 0 and 5, i.e., n is 0, 1, 2, 3, 4, and 5. In a particular embodiment, n is an integer comprised between 0 and 3. Preferably, n is 0, 1, or 2. In a further particular embodiment, n is an integer comprised between 1 and 5, i.e., n is 1, 2, 3, 4, and 5. Preferably, n is an integer comprised between 1 and 3. More preferably n is 1 or 2, even more preferably 1.

According to the invention, each R₂ is independently a radical selected in the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro.

In a particular embodiment, each R₂ is independently a radical selected in the group consisting of a (C₁-C₆)alkyl optionally substituted by at least one halogen, a (C₁-C₆)alkyloxy, optionally substituted by at least one halogen, a halogen, a nitrile, and a nitro.

In a further particular embodiment, each R₂ is independently a radical selected in the group consisting of a (C₁-C₆)alkyl optionally substituted by at least one halogen, a (C₁-C₆)alkyloxy, a halogen, a nitro, and a nitrile. In an even more particular embodiment, each R₂ is independently a radical selected in the group consisting of a methyl, a trifluoromethyl, an isopropyl, a methoxy, a fluorine, a bromine, or a chlorine, a nitro, and a nitrile.

In a preferred embodiment, n is 1 and R₂ is a radical selected in the group consisting of a (C₁-C₆)alkyl optionally substituted by at least one halogen, preferably a methyl an isopropyl, or an isopropyl, a (C₁-C₆)alkyloxy, preferably a methoxy, a halogen, preferably a bromine or a chlorine, a nitrile, and a nitro.

In a further preferred embodiment, n is 2 and each R₂ is independently a radical selected in the group consisting of a halogen, preferably a fluorine or a chlorine

In a further preferred embodiment, n is 3 and each R₂ is a methoxy.

In a particular embodiment, X is —(CH₂)—, and R₁, n and R₂ are such as defined above.

According to this particular embodiment, R₁ is a phenyl or a pyridinyl group, preferably a phenyl, said phenyl or pyridinyl groups are optionally substituted by at least one radical selected in the group consisting of a (C₁-C₆)alkyl optionally substituted by at least one halogen, a (C₁-C₆)alkyloxy optionally substituted by at least one halogen, a halogen, and a —CO₂R₃ with R₃ being a hydrogen or a (C₁-C₆)alkyl. More particularly, R₁ is a phenyl or a pyridinyl group, preferably a phenyl group, said phenyl or pyridinyl groups are optionally substituted by at least one radical selected in the group consisting of a methoxy, a methyl, a halogen, preferably a chlorine or a bromine, and a —CO₂R₃ with R₃ being a methyl.

According to this particular embodiment, n is an integer comprised between 0 and 3. Preferably n is 0, 1, or 2. More preferably n is 1 or 2, even more preferably 1. Each R₂ is independently a radical selected in the group consisting of a (C₁-C₆)alkyl optionally substituted by at least one halogen, a (C₁-C₆)alkyloxy, optionally substituted by at least one halogen, a halogen, a nitrile, and a nitro. Preferably, R₂ is independently a radical selected in the group consisting of a methyl, a trifluoromethyl, an isopropyl, a methoxy, a fluorine, a bromine, or a chlorine, a nitro, and a nitrile.

In a preferred embodiment of the invention, a compound of formula (I), (IA), or (IB) is such that:
X is —(CH₂)—;
R₁ is a phenyl or a pyridinyl group, preferably a phenyl, said phenyl or pyridinyl groups are optionally substituted by at least one radical selected in the group consisting of a methyl, a chlorine, a bromine, and a —CO₂R₃ with R₃ being a methyl;
n is 1; and
R₂ is a radical selected in the group consisting of an isopropyl, and a bromine.

In a further particular embodiment, X is —(CH₂)—CO—, and R₁, n and R₂ are such as defined above.

According to this particular embodiment, R₁ is a phenyl or a pyridinyl group, said phenyl or pyridinyl groups are optionally substituted by at least one radical selected in the group consisting of a (C₁-C₆)alkyl optionally substituted by at least one halogen, a (C₁-C₆)alkyloxy optionally substituted by at least one halogen, a halogen, and a —CO₂R₃ with R₃ being a hydrogen or a (C₁-C₆)alkyl. Preferably, R₁ is a phenyl or a pyridinyl group.

According to this particular embodiment, n is an integer comprised between 0 and 3. Preferably n is 0, 1, or 2. More preferably n is 0 or 1. Each R₂ is independently a radical selected in the group consisting of a (C₁-C₆)alkyl optionally substituted by at least one halogen, a (C₁-C₆)alkyloxy, optionally substituted by at least one halogen, a halogen, a nitrile, and a nitro. Preferably, n is 0 or 1 and R₂ is a nitro.

In a further particular embodiment, X is —(CH₂)—(CH₂)—, and R₁, n and R₂ are such as defined above.

According to this particular embodiment, R₁ is a phenyl or a pyridinyl group, said phenyl or pyridinyl groups are optionally substituted by at least one radical selected in the group consisting of a (C₁-C₆)alkyl optionally substituted by at least one halogen, a (C₁-C₆)alkyloxy optionally substituted by at least one halogen, a halogen, and a —CO₂R₃ with R₃ being a hydrogen or a (C₁-C₆)alkyl. Preferably, R₁ is a phenyl group.

According to this particular embodiment, n is an integer comprised between 0 and 3. Preferably n is 0, 1, or 2. More preferably n is 0. Each R₂ is independently a radical selected in the group consisting of a (C₁-C₆)alkyl optionally substituted by at least one halogen, a (C₁-C₆)alkyloxy, optionally substituted by at least one halogen, a halogen, a nitrile, and a nitro.

In a preferred embodiment, a compound of formula (I), (IA), or (IB) as above defined is selected in the group consisting of:
MCK248: 1-benzyl-N⁵-phenyl-1H-1,2,4-triazole-3,5-diamine;
MCK249: 1-benzyl-N³-phenyl-1H-1,2,4-triazole-3,5-diamine;
MCK250: 1-(2-methylbenzyl)-N³-phenyl-1H-1,2,4-triazole-3,5-diamine;
MCK253: 1-benzyl-N³-(pyridin-2-yl)-1H-1,2,4-triazole-3,5-diamine;
MCK254: 1-benzyl-N³-(pyridin-3-yl)-1H-1,2,4-triazole-3,5-diamine;
MCK257: 1-(2,6-difluorobenzyl)-N⁵-phenyl-1H-1,2,4-triazole-3,5-diamine;
MCK259: 1-(2,6-difluorobenzyl)-N³-(pyridin-2-yl)-1H-1,2,4-triazole-3,5-diamine;
MCK260: 1-(3-methoxybenzyl)-N⁵-phenyl-1H-1,2,4-triazole-3,5-diamine;
MCK261: 1-(3-methoxybenzyl)-N³-phenyl-1H-1,2,4-triazole-3,5-diamine;
MCK262: 1-(4-isopropylbenzyl)-N⁵-phenyl-1H-1,2,4-triazole-3,5-diamine;
MCK263: 1-(4-isopropylbenzyl)-N³-phenyl-1H-1,2,4-triazole-3,5-diamine;
MCK264: 1-(3-bromobenzyl)-N³-phenyl-1H-1,2,4-triazole-3,5-diamine;
MCK268: 1-(2,6-dichlorobenzyl)-N³-phenyl-1H-1,2,4-triazole-3,5-diamine;
MCK269: 1-(2-methylbenzyl)-N³-(pyridin-2-yl)-1H-1,2,4-triazole-3,5-diamine;
MCK271: 1-(4-methylbenzyl)-N³-phenyl-1H-1,2,4-triazole-3,5-diamine;
MCK272: 1-(4-chlorobenzyl)-N³-phenyl-1H-1,2,4-triazole-3,5-diamine;
MCK273: N³-phenyl-1-(3-(trifluoromethyl)benzyl)-1H-1,2,4-triazole-3,5-diamine;
MCK274: 1-(3,5-difluorobenzyl)-N³-phenyl-1H-1,2,4-triazole-3,5-diamine;
MCK275: 1-(3-nitrobenzyl))-N³-phenyl-1H-1,2,4-triazole-3,5-diamine;
MCK276: 1-(3-bromobenzyl)-N³-(pyridin-2-yl)-1H-1,2,4-triazole-3,5-diamine;
MCK278: 1-(2,6-dichlorobenzyl)-N³-(pyridin-2-yl)-1H-1,2,4-triazole-3,5-diamine;
MCK279: 1-(4-methylbenzyl)-N³-(pyridin-2-yl)-1H-1,2,4-triazole-3,5-diamine;
MCK280: 1-(4-chlorobenzyl)-N³-(pyridin-2-yl)-1H-1,2,4-triazole-3,5-diamine;
MCK281: 4-((5-amino-3-(pyridin-2-ylamino)-1H-1,2,4-triazol-1-yl)methyl)benzonitrile;
MCK282: N³-(pyridin-2-yl)-1-(3-(trifluoromethyl)benzyl)-1H-1,2,4-triazole-3,5-diamine;
MCK283: 1-(3,5-difluorobenzyl)-N³-(pyridin-2-yl)-1H-1,2,4-triazole-3,5-diamine;
MCK284: 1-(2,4-difluorobenzyl)-N³-(pyridin-2-yl)-1H-1,2,4-triazole-3,5-diamine;
MCK288: 1-(4-nitrobenzyl))-N³-phenyl-1H-1,2,4-triazole-3,5-diamine;
MCK289: 1-(2-nitrobenzyl)-N³-(pyridin-2-yl)-1H-1,2,4-triazole-3,5-diamine;
MCK290: 1-(4-nitrobenzyl)-N³-(pyridin-2-yl)-1H-1,2,4-triazole-3,5-diamine;
MCK292: 1-(3-nitrobenzyl)-N³-(pyridin-2-yl)-1H-1,2,4-triazole-3,5-diamine;
MCK295: 2-(5-amino-3-(pyridin-2-ylamino)-1H-1,2,4-triazol-1-yl)-1-(3-nitrophenyl)ethan-1-one;
MCK297: 1-(2-nitrobenzyl)-N³-phenyl-1H-1,2,4-triazole-3,5-diamine;
MCK298: N³-phenyl-1-(3,4,5-trimethoxybenzyl)-1H-1,2,4-triazole-3,5-diamine;
MCK299: 2-(5-amino-3-(phenylamino)-1H-1,2,4-triazol-1-yl)-1-phenylethan-1-one;
MCK300: 1-phenethyl-N⁵-phenyl-1H-1,2,4-triazole-3,5-diamine;
MCK301: 1-phenethyl-N³-phenyl-1H-1,2,4-triazole-3,5-diamine;
MCK338: N⁵-phenyl-1-(3-(trifluoromethyl)benzyl)-1H-1,2,4-triazole-3,5-diamine;
MCK339: 1-(3-bromobenzyl)-N³-phenyl-1H-1,2,4-triazole-3,5-diamine;
MCK340: 1-(3-bromobenzyl)-N³-(4-methoxyphenyl)-1H-1,2,4-triazole-3,5-diamine;
MCK341: 1-(3-bromobenzyl)-N⁵-(4-methylphenyl)-1H-1,2,4-triazole-3,5-diamine;
MCK342: 1-(3-bromobenzyl)-N³-(4-methylphenyl)-1H-1,2,4-triazole-3,5-diamine;
MCK343: 1-(3-bromobenzyl)-N⁵-(4-chlorophenyl)-1H-1,2,4-triazole-3,5-diamine;
MCK344: 1-(3-bromobenzyl)-N³-(4-chlorophenyl)-1H-1,2,4-triazole-3,5-diamine;
MCK345: 1-(3-bromobenzyl)-N⁵-(4-bromophenyl)-1H-1,2,4-triazole-3,5-diamine;
MCK346: 1-(3-bromobenzyl)-N³-(4-bromophenyl)-1H-1,2,4-triazole-3,5-diamine;
MCK347: methyl-4-((3-amino-1-(3-bromobenzyl)-1H-1,2,4-triazol-5-yl)amino)benzoate;
MCK348: methyl-4-((5-amino-1-(3-bromobenzyl)-1H-1,2,4-triazol-3-yl)amino)benzoate;
MCK349: 1-(3-bromobenzyl)-N³-(3,4-dichlorophenyl)-1H-1,2,4-triazole-3,5-diamine;
MCK350: N³-(3,4-dichlorophenyl)-1-(4-isopropylbenzyl)-1H-1,2,4-triazole-3,5-diamine;
MCK351: 1-(3-bromobenzyl)-N³-(pyridin-3-yl)-1H-1,2,4-triazole-3,5-diamine; and
MCK352: 1-(3-bromobenzyl)-N⁴-(pyridin-3-yl)-1H-1,2,4-triazole-3,5-diamine.

In a more preferred embodiment, a compound of formula (I), (IA), or (IB) as above defined is selected in the group consisting of:
MCK250: 1-(2-methylbenzyl)-N³-phenyl-1H-1,2,4-triazole-3,5-diamine;
MCK257: 1-(2,6-difluorobenzyl)-N⁵-phenyl-1H-1,2,4-triazole-3,5-diamine;
MCK259: 1-(2,6-difluorobenzyl)-N³-(pyridin-2-yl)-1H-1,2,4-triazole-3,5-diamine;
MCK260: 1-(3-methoxybenzyl)-N⁵-phenyl-1H-1,2,4-triazole-3,5-diamine;
MCK261: 1-(3-methoxybenzyl)-N³-phenyl-1H-1,2,4-triazole-3,5-diamine;
MCK262: 1-(4-isopropylbenzyl)-N⁵-phenyl-1H-1,2,4-triazole-3,5-diamine;
MCK263: 1-(4-isopropylbenzyl)-N³-phenyl-1H-1,2,4-triazole-3,5-diamine;
MCK264: 1-(3-bromobenzyl)-N²-phenyl-1H-1,2,4-triazole-3,5-diamine;
MCK268: 1-(2,6-dichlorobenzyl)-N³-phenyl-1H-1,2,4-triazole-3,5-diamine;
MCK269: 1-(2-methylbenzyl)-N³-(pyridin-2-yl)-1H-1,2,4-triazole-3,5-diamine;
MCK271: 1-(4-methylbenzyl)-N³-phenyl-1H-1,2,4-triazole-3,5-diamine;
MCK272: 1-(4-chlorobenzyl)-N³-phenyl-1H-1,2,4-triazole-3,5-diamine;
MCK273: N²-phenyl-1-(3-(trifluoromethyl)benzyl)-1H-1,2,4-triazole-3,5-diamine;
MCK274: 1-(3,5-difluorobenzyl)-N³-phenyl-1H-1,2,4-triazole-3,5-diamine;
MCK275: 1-(3-nitrobenzyl))-N³-phenyl-1H-1,2,4-triazole-3,5-diamine;
MCK276: 1-(3-bromobenzyl)-N³-(pyridin-2-yl)-1H-1,2,4-triazole-3,5-diamine;
MCK278: 1-(2,6-dichlorobenzyl)-N³-(pyridin-2-yl)-1H-1,2,4-triazole-3,5-diamine;
MCK279: 1-(4-methylbenzyl)-N³-(pyridin-2-yl)-1H-1,2,4-triazole-3,5-diamine;
MCK280: 1-(4-chlorobenzyl)-N³-(pyridin-2-yl)-1H-1,2,4-triazole-3,5-diamine;
MCK281: 4-((5-amino-3-(pyridin-2-ylamino)-1H-1,2,4-triazol-1-yl)methyl)benzonitrile;
MCK282: N³-(pyridin-2-yl)-1-(3-(trifluoromethyl)benzyl)-1H-1,2,4-triazole-3,5-diamine;
MCK283: 1-(3,5-difluorobenzyl)-N³-(pyridin-2-yl)-1H-1,2,4-triazole-3,5-diamine;
MCK284: 1-(2,4-difluorobenzyl)-N³-(pyridin-2-yl)-1H-1,2,4-triazole-3,5-diamine;
MCK288: 1-(4-nitrobenzyl))-N³-phenyl-1H-1,2,4-triazole-3,5-diamine;
MCK289: 1-(2-nitrobenzyl)-N³-(pyridin-2-yl)-1H-1,2,4-triazole-3,5-diamine;
MCK290: 1-(4-nitrobenzyl)-N³-(pyridin-2-yl)-1H-1,2,4-triazole-3,5-diamine;
MCK292: 1-(3-nitrobenzyl)-N³-(pyridin-2-yl)-1H-1,2,4-triazole-3,5-diamine;
MCK295: 2-(5-amino-3-(pyridin-2-ylamino)-1H-1,2,4-triazol-1-yl)-1-(3-nitrophenyl)ethan-1-one;
MCK297: 1-(2-nitrobenzyl)-N³-phenyl-1H-1,2,4-triazole-3,5-diamine;
MCK298: N³-phenyl-1-(3,4,5-trimethoxybenzyl)-1H-1,2,4-triazole-3,5-diamine;
MCK338: N⁵-phenyl-1-(3-(trifluoromethyl)benzyl)-1H-1,2,4-triazole-3,5-diamine;
MCK339: 1-(3-bromobenzyl)-N⁵-phenyl-1H-1,2,4-triazole-3,5-diamine;
MCK340: 1-(3-bromobenzyl)-N³-(4-methoxyphenyl)-1H-1,2,4-triazole-3,5-diamine;
MCK341: 1-(3-bromobenzyl)-N⁵-(4-methylphenyl)-1H-1,2,4-triazole-3,5-diamine;
MCK342: 1-(3-bromobenzyl)-N³-(4-methylphenyl)-1H-1,2,4-triazole-3,5-diamine;
MCK343: 1-(3-bromobenzyl)-N⁵-(4-chlorophenyl)-1H-1,2,4-triazole-3,5-diamine;
MCK344: 1-(3-bromobenzyl)-N³-(4-chlorophenyl)-1H-1,2,4-triazole-3,5-diamine;
MCK345: 1-(3-bromobenzyl)-N⁵-(4-bromophenyl)-1H-1,2,4-triazole-3,5-diamine;
MCK346: 1-(3-bromobenzyl)-N³-(4-bromophenyl)-1H-1,2,4-triazole-3,5-diamine;
MCK347: methyl-4-((3-amino-1-(3-bromobenzyl)-1H-1,2,4-triazol-5-yl)amino)benzoate;
MCK348: methyl-4-((5-amino-1-(3-bromobenzyl)-1H-1,2,4-triazol-3-yl)amino)benzoate;
MCK349: 1-(3-bromobenzyl)-N³-(3,4-dichlorophenyl)-1H-1,2,4-triazole-3,5-diamine;
MCK350: N³-(3,4-dichlorophenyl)-1-(4-isopropylbenzyl)-1H-1,2,4-triazole-3,5-diamine;
MCK351: 1-(3-bromobenzyl)-N³-(pyridin-3-yl)-1H-1,2,4-triazole-3,5-diamine; and
MCK352: 1-(3-bromobenzyl)-N⁴-(pyridin-3-yl)-1H-1,2,4-triazole-3,5-diamine.

In an even more preferred embodiment, a compound of formula (I), (IA), or (IB) as above defined is selected in the group consisting of:
MCK263: 1-(4-isopropylbenzyl)-N³-phenyl-1H-1,2,4-triazole-3,5-diamine;
MCK264: 1-(3-bromobenzyl)-N³-phenyl-1H-1,2,4-triazole-3,5-diamine;
MCK342: 1-(3-bromobenzyl)-N³-(4-methylphenyl)-1H-1,2,4-triazole-3,5-diamine;
MCK343: 1-(3-bromobenzyl)-N⁵-(4-chlorophenyl)-1H-1,2,4-triazole-3,5-diamine;
MCK344: 1-(3-bromobenzyl)-N³-(4-chlorophenyl)-1H-1,2,4-triazole-3,5-diamine;
MCK345: 1-(3-bromobenzyl)-N⁵-(4-bromophenyl)-1H-1,2,4-triazole-3,5-diamine;
MCK348: methyl-4-((5-amino-1-(3-bromobenzyl)-1H-1,2,4-triazol-3-yl)amino)benzoate;
MCK349: 1-(3-bromobenzyl)-N³-(3,4-dichlorophenyl)-1H-1,2,4-triazole-3,5-diamine;
MCK350: N³-(3,4-dichlorophenyl)-1-(4-isopropylbenzyl)-1H-1,2,4-triazole-3,5-diamine; and
MCK352: 1-(3-bromobenzyl)-N⁴-(pyridin-3-yl)-1H-1,2,4-triazole-3,5-diamine.

### Application

According to the present invention, the terms below have the following meanings:

As used herein, the terms “treatment”, “treat” or “treating” refer to any act intended to ameliorate the health status of patients such as therapy, prevention, prophylaxis and retardation of a disease, particularly an ocular or eye disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refer to a component of a pharmaceutical composition having a therapeutic effect. Particularly, such terms designate a compound of formula (I), (IA) or (IB).

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder, particularly an ocular or eye disease.

As used herein, the term “effective amount” refers to a quantity of an active ingredient or of a pharmaceutical composition that prevents, removes or reduces the deleterious effects of the disease, particularly an ocular or eye disease. It is obvious that the quantity to be administered can be adapted by the man skilled in the art according to the subject to be treated, to the nature of the disease, etc. In particular, doses and regimen of administration may be adapted to the nature, the stage and the severity of the disease to be treated, as well as the weight, the age and the global health of the subject to be treated, as well as the judgment of the doctor.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

As illustrated by examples, the inventors have demonstrated the therapeutic interest of the compounds of the invention.

Accordingly, the present invention relates to a pharmaceutical or veterinary composition comprising a compound of formula (I), (IA), or (IB) according to the invention or a pharmaceutically acceptable salt thereof. Preferably, the pharmaceutical composition further comprises a pharmaceutically or veterinary acceptable carrier or excipient. The present invention relates to the use of a compound of formula (I), (IA), or (IB) according to the invention as a drug or a medicine. The invention further relates to a method for treating a disease in a subject, wherein a therapeutically effective amount of a compound of formula (I), (IA), or (IB) according to the invention, is administered to said subject in need thereof. The invention also relates to the use of a compound of formula (I), (IA), or (IB) according to the invention, for the manufacture of a medicine, a drug, or a medicament. The invention also relates to a pharmaceutical composition comprising a compound of formula (I), (IA), or (IB) according to the invention for use as a drug.

The present invention further relates to a compound of formula (I), (IA), or (IB) as defined herein, or a pharmaceutically acceptable salt or a tautomer thereof, for use for treating an ocular disease.

The present invention further relates to a method for treating an ocular disease, comprising administering in a subject in need thereof an effective amount of a compound of formula (I), (IA), or (IB) as defined herein, or a pharmaceutically acceptable salt thereof.

The present invention also relates to a use of a compound of formula (I), (IA), or (IB) as defined herein, or a pharmaceutically acceptable thereof, for the manufacture of a drug, a medicine, or a medicament, for treating an ocular disease.

The present invention further relates to a pharmaceutical or veterinary composition comprising a compound of formula (I), (IA), or (IB) as defined herein, a pharmaceutically acceptable salt thereof, for use for treating an ocular disease.

The present invention further relates to a method for treating an ocular disease, comprising administering in a subject in need thereof an effective amount of a pharmaceutical composition comprising a compound of formula (I), (IA), or (IB) as defined herein, or a pharmaceutically acceptable salt thereof.

The present invention also relates to a use of a compound of formula (I), (IA), or (IB) as defined herein, or a pharmaceutically acceptable salt thereof, for the manufacture of a drug, a medicament, or a pharmaceutical composition for treating an ocular disease.

In a particular embodiment, the ocular disease is an ocular disease associated with angiogenesis. The present invention thus relates to a compound of formula (I), (IA), or (IB) as defined herein or a pharmaceutically acceptable salt thereof or a pharmaceutical or veterinary composition comprising such a compound, for use for treating an ocular disease associated with angiogenesis.

The present invention further relates to a method for treating an ocular disease associated with angiogenesis, comprising administering in a subject in need thereof an effective amount of a compound of formula (I), (IA), or (IB) as defined herein or a pharmaceutically acceptable salt thereof or a pharmaceutical or veterinary composition comprising such a compound.

The present invention also relates to a use of a compound of formula (I), (IA), or (IB) as defined herein, or a pharmaceutically acceptable salt thereof, for the manufacture of a drug, a medicament, or a pharmaceutical or veterinary composition for treating an ocular disease associated with angiogenesis.

In a particular embodiment, the ocular disease is chosen among corneal graft angiogenesis, neovascular glaucoma, diabetic retinopathy, corneal diseases induced by new blood vessels, macular degeneration, pterygium, retinal degeneration, retrolental fibroplasia, granular conjunctivitis, macular edema or Stargardt's disease. Preferably, the ocular disease is chosen among diabetic retinopathy, macular degeneration, neovascular glaucoma, macular edema or Stargardt's disease.

An object of the invention is also a compound of formula (I), (IA), or (IB) as defined herein or a pharmaceutically acceptable salt thereof or a pharmaceutical or veterinary composition comprising such a compound, for use for treating an ocular disease chosen among diabetic retinopathy, macular degeneration, neovascular glaucoma, macular edema or Stargardt's disease.

The present invention further relates to a method for treating an ocular disease chosen among diabetic retinopathy, macular degeneration, neovascular glaucoma, macular edema or Stargardt's disease, comprising administering in a subject in need thereof an effective amount of a compound of formula (I), (IA), or (IB) as defined herein or a pharmaceutically acceptable salt thereof or a pharmaceutical or veterinary composition comprising such a compound.

The present invention also relates to a use of a compound of formula (I), (IA), or (IB) as defined herein, or a pharmaceutically acceptable salt thereof, for the manufacture of a drug, a medicament, or a pharmaceutical or veterinary composition for treating an ocular disease chosen among diabetic retinopathy, macular degeneration, neovascular glaucoma, macular edema or Stargardt's disease.

The route of administration can be ocular topical (i.e., topical ophthalmic instillation), transdermal, oral, rectal, sublingual, intranasal, intrathecal, intraocular or parenteral (including subcutaneous, intramuscular, intraperitoneal, intravenous and/or intradermal). Preferably, the route of administration is topical, oral or parenteral. Even more preferably, the route of administration is topical or oral. Still more preferably, the route of administration is topical.

Advantageously, the route of administration route is ocular topical. The pharmaceutical composition is suitable for one or more of the above routes.

The pharmaceutical or veterinary composition may be formulated as solutions in pharmaceutically acceptable solvents or as emulsions, suspensions or dispersions in suitable pharmaceutical solvents or carriers, or as pills, tablets or capsules that contain solid vehicles in a way known in the art. Formulations of the present invention suitable for oral administration may be in the form of individual units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations suitable for parenteral administration include, for instance, a sterile oily or aqueous preparation of the active ingredient which is preferably isotonic with the blood of the recipient. Formulations for topical application may be in the form of cream, lotion, ointment, solution, suspension, gel, in the form of an oil-in-water emulsion or a water-in-oil emulsion. Any such formulation can also contain other pharmaceutically compatible and nontoxic auxiliary agents, such as stabilizers, antioxidants, binders, dyes, emulsifiers or flavoring substances. The formulations of the present invention comprise an active ingredient in association with a pharmaceutically acceptable carrier, and optionally other therapeutic ingredients. The carrier must be “acceptable” in the sense that it is compatible with the other ingredients of the formulations and not deleterious to the recipient thereof.

In a preferred embodiment, the pharmaceutical or veterinary compositions according to the invention are formulated to be administered via a topical route, preferably an ocular topical route, typically via topical ophthalmic instillation. Preferably, the pharmaceutical or veterinary compositions according to the invention are in a form of a gel, a suspension, a solution or a sustained release device.

In a particular embodiment, the pharmaceutical or veterinary composition according to the invention is coated on an implant. Said implant dissolves naturally and slowly releases the pharmaceutical or veterinary composition over time, thereby reducing the daily need of multiple eye drops. Advantageously, the implant does not need to be removed from the patient.

Further aspects and advantages of the present invention will be described in the following examples, which should be regarded as illustrative and not limiting.

### EXAMPLES

### Example A: Chemistry

### General Information

All the commercially available products from chemical providers were used without purification. Solvents were purchased from Sigma Aldrich. All chemicals were purchased from Aldrich, Fisher or Alfa Aesar. All the chemical reactions were monitored simultaneously by LCMS, HPLC and thin-layer chromatography (TLC, Merck silica gel 60 F254 plates) and visualized both by UV radiation (254 & 365 nm) and by spraying with a relevant staining agent followed by subsequent warming with a heat gun. Column chromatography was performed on a CombiFlash Rf+, TELEDYNE Isco, using prepacked column (CHROMABOND Flash, SiOH 40-63 μm, MACHEREY NAGEL or CHROMABOND Flash, C₁₈ec 40-63 μm, MACHEREY NAGEL or POLYGOPREP 60-30 Cis, MACHEREY NAGEL). All NMR spectra (H, ¹³C, ¹⁹F) were recorded on Bruker Advance Spectrometers (200 or 400 MHz). ¹H NMR (200 and 400 MHz), ¹³C{1H} NMR (50 and 101 MHz) and ¹⁹F{1H} (188 and 377 MHz) spectra were obtained with samples dissolved in CDCl₃ and DMSO-d₆ with the solvent residual signals as internal references: CHCl₃ (¹H=7.26 ppm, ¹³C=77.16) and DMSO-d₆ (¹H=2.50 ppm, ¹³C=39.52). Chemical shifts (d) are given in ppm to the nearest 0.01 (¹H) or 0.1 ppm (¹³C). The coupling constants (J) are given in Hertz (Hz). The signals are reported as follows: chemical shift, multiplicity (s=singlet, d=doublet, t=triplet, m=multiplet, dd=doublet of doublets, br s=broad singlet), coupling constants (J) and integration. Dye syntheses were performed in a Branson 5510 ultrasound bath. Mass spectra (ESI-MS) were recorded on a ThermoFisher LCQ advantage ion trap mass spectrometer hyphenated with an Agilent 1100 HPLC or an Agilent iQ single quadrupole mass spectrometer hyphenated with an agilent 1260 infinity HPLC. HRMS was carried out on a Thermo Q-exactive Focus mass spectrometer hyphenated to Thermo Vanquish UHPLC system including degasser, binary pump, autosampler and multiwavelength detector. Separation was done on a Thermo scientific Hypersil GOLD (150 mm×2.1 mm, 1.9 μm) at 0.2 ml/min. Gradient starts at 90/10 water (0.1% formic acid)/acetonitrile (0.1% formic acid) to 98% acetonitrile (0.1% formic acid) in 15 minutes, and then kept for two minutes. Mass spectrometry was used in Electrospray ionization in switch mode (alternate positive/negative scan) using the following parameters: spray voltage was 3.7 kV (pos mode) and 2.7 kV (neg mode); capillary temperature: 320° C.; sheath gas: 30 a.u.; Auxiliary gas: 15 a.u.; probe heater temperature: 350° C. Melting points were achieved over system Kofler type WME Heizbank nr. 7563 (44-266° C.). The purity of compounds was further assayed by HPLC analysis on a JASCO PU-2089 apparatus with Supelco analytical column Ascentis Express C18, 100 mm×46 mm 5 μM. Eluent A: water with 1‰ formic acid, Eluent B: CH₃CN with 1‰ formic acid. Four different methods were used which are described as follows: Method 1: 100% A over 2 min, 100% A to 60% A over 5 min, 60% A for 3 min then from 60% A to 100% A over 1 min (11 min in total). Method 2: 100% A over 2 min, 100% A to 60% A over 8 min, 60% A for 4 min then from 60% A to 100% A over 1 min (15 min in total)). Method 3: 95% A over 2 min, 95% A to 50% A over 8 min, 50% A for 4 min, from 50% A to 40% A over 6.5 min, 40% A for 6 min, from 40% A to 95% for 2 min then 95% A for 2.5 min (30 min in total)). Method 4: 95% A over 2 min, 95% A to 50% A over 8 min, 50% A for 8 min, from 50% A to 95% A over 0.5 min, then 95% A for 0.5 min (20 min in total).

### MCK248: 1-benzyl-N⁵-phenyl-1H-1,2,4-triazole-3,5-diamine

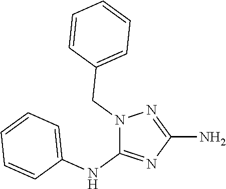 [SMILES: Nc1nc(Nc2ccccc2)n(Cc2ccccc2)n1]

N³-phenyl-1H-1,2,4-triazole-3,5-diamine (200 mg, 1.14 mmol) was dissolved in degassed DMF, then Cs₂CO₃ (447 mg, 1.37 mmol) was added. Benzyl chloride (0.14 mL, 1.26 mmol) was added and the reaction mixture was stirred at r.t. overnight. Cs₂CO₃ was filtered and DMF was evaporated under reduced pressure. Purification by silica gel flash chromatography (CH₂Cl₂/THF, 10/0 then 5/5) afforded the titled compound as a brown solid (20.9 mg, 0.0786 mmol, 7% yield) along with the N₃-benzylated regioisomer. Brown solid, 20.9 mg, 0.0786 mmol, 7% yield. Rf (CH₂Cl₂/THF, 1/1, v/v)=0.69. M.p.=166-168° C. ¹H NMR (400 MHz, DMSO-d₆) δ 8.81 (s, 1H, NH), 7.59 (d, J=7.7 Hz, 2H, CH_{Ar}), 7.34 (t, J=7.3 Hz, 2H, CH_{Ar}), 7.29-7.22 (m, 3H, CH_{Ar}), 7.22-7.17 (m, 2H, CH_{Ar}), 6.88 (t, J=7.3 Hz, 1H, CH_{Ar}), 5.14 (s, 2H, NH₂), 5.11 (s, 2H, CH₂). ¹³C NMR (101 MHz, DMSO-d₆) δ 160.7, 150.2, 141.1, 137.6, 128.6 (2C), 128.4 (2C), 127.2, 127.2 (2C), 120.5, 116.9 (2C), 48.7. LCMS: t_{R}=9.56 min. [M+H]⁺=266.07. HPLC (λ₂₅₄): Purity 97.7%; HPLC (λ₂₈₀): Purity 96.8%; t_{R}: 7.73 min (method 1).

### MCK249: 1-benzyl-N³-phenyl-1H-1,2,4-triazole-3,5-diamine

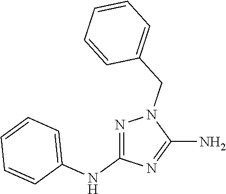 [SMILES: Nc1nc(Nc2ccccc2)nn1Cc1ccccc1]

N³-phenyl-1H-1,2,4-triazole-3,5-diamine (150 mg, 0.857 mmol) was dissolved in degassed DMF (1 mL), then potassium tert-butoxide (96 mg, 0.857 mmol) was added. Benzyl chloride (0.12 mL, 0.857 mmol), dissolved in degassed DMF (1 mL), was added dropwise and the reaction mixture was stirred at 0° C. for 1 h, then at r.t. for an additional 1 h. Water was added to the mixture reaction and was extracted with ethyl acetate, washed with water, dried over magnesium sulfate and the solvents were evaporated under reduced pressure. Purification by reversed-phase chromatography (H₂O/CH₃CN, 10/0 then 60/40) afforded the titled compound as a white solid (119 mg, 0.449 mmol, 50%) along with the N₅-benzylated regioisomer (15.9 mg, 0.06 mmol, 7%). White solid, 119 mg, 0.449 mmol, 50% yield. Rf (CH₂Cl₂/THF, 1/1, v/v)=0.54. M.p.=136-138° C. ¹H NMR (400 MHz, DMSO-d₆) δ 8.67 (s, 1H, NH), 7.46 (d, J=7.8 Hz, 2H, CH_{Ar}), 7.34 (t, J=7.3 Hz, 2H, CH_{Ar}), 7.29-7.21 (m, 3H, CH_{Ar}), 7.14 (t, J=7.9 Hz, 2H, CH_{Ar}), 6.70 (t, J=7.3 Hz, 1H, CH_{Ar}), 6.29 (s, 2H, NH₂), 5.03 (s, 2H, CH₂). ¹³C NMR (101 MHz, DMSO-d₆) δ 157.1, 154.1, 142.4, 137.6, 128.4 (2C), 128.4 (2C), 127.2 (2C), 127.2, 118.4, 115.5 (2C), 48.7. LCMS: t_{R}=9.65 min. [M+H]⁺=266.07. HPLC (λ₂₅₄): Purity 99.4%; HPLC (λ₂₈₀): Purity 97.9%; t_{R}: 8.25 min (method 1).

### MCK250: 1-(2-methylbenzyl)-N³-phenyl-1H-1,2,4-triazole-3,5-diamine

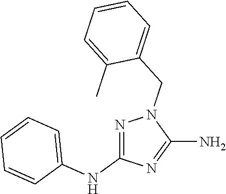 [SMILES: Cc1ccccc1Cn1nc(Nc2ccccc2)nc1N]

N³-phenyl-1H-1,2,4-triazole-3,5-diamine (150 mg, 0.857 mmol) was dissolved in degassed DMF, then potassium tert-butoxide (96 mg, 0.857 mmol) was added. 2-Methylbenzyl chloride (150 mg, 1.07 mmol) was added and the reaction mixture was stirred at r.t. for 2 h. Water was added to the mixture reaction and was extracted with ethyl acetate, washed with water, dried over magnesium sulfate and the solvents were evaporated under reduced pressure. Purification by reversed-phase chromatography (H₂O/CH₃CN, 10/0 then 60/40) afforded the titled compound as a brown solid (111 mg, 0.398 mmol, 46% yield). Brown solid, 111 mg, 0.398 mmol, 46% yield. Rf (CH₂Cl₂/THF, 1/1, v/v)=0.51. M.p.=156-158° C. ¹H NMR (400 MHz, DMSO-d₆) δ 8.67 (s, 1H, NH), 7.48-7.41 (m, 2H, CH_{Ar}), 7.14 (ddd, J=15.8, 13.8, 7.6 Hz, 5H, CH_{Ar}), 6.82 (d, J=6.8 Hz, 1H, CH_{Ar}), 6.68 (t, J=7.3 Hz, 1H, CH_{Ar}), 6.23 (s, 2H, NH₂), 4.99 (s, 2H, CH₂), 2.33 (s, 3H, CH₃). ¹³C NMR (101 MHz, DMSO-d₆) δ 157.1, 154.3, 142.4, 135.8, 135.4, 129.9, 128.4 (2C), 126.9, 126.6, 125.8, 118.4, 115.5 (2C), 46.9, 18.7. LCMS: t_{R}=10.10 min. [M+H]⁺=280.13. HPLC (λ₂₅₄): Purity 99.5%; HPLC (λ₂₈₀): Purity 98.5%; t_{R}: 8.78 min (method 1).

### MCK253: 1-benzyl-N³-(pyridin-2-yl)-1H-1,2,4-triazole-3,5-diamine

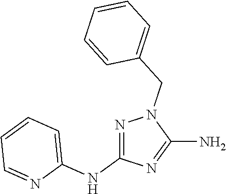 [SMILES: Nc1nc(Nc2ccccn2)nn1Cc1ccccc1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. Benzyl chloride (0.16 mL, 1.42 mmol), dissolved in degassed DMF (5 mL), was added dropwise and the reaction mixture was stirred at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (140 mg, 0.526 mmol, 37% yield). Yellow solid, 16.4 mg, 0.062 mmol, 2% yield. Rf (CH₂Cl₂/MeOH:9/1, v/v)=0.61. M.p.=204° C. ¹H NMR (400 MHz, DMSO-d₆) δ 9.48 (s, 1H, NH), 8.16-8.11 (m, 1H, CH_{Pyr}), 7.83 (d, J=8.5 Hz, 1H, CH_{Pyr}), 7.64-7.57 (m, 1H, CH_{Pyr}), 7.35 (t, J=7.3 Hz, 2H, CH_{Ph}), 7.30-7.20 (m, 3H, CH_{Ph}), 6.80-6.72 (m, 1H, CH_{Pyr}), 6.47 (s, 2H, NH₂), 5.06 (s, 2H, CH₂). ¹³C NMR (101 MHz, DMSO-d₆) δ 155.8, 154.4, 154.3, 147.6, 137.6, 137.4, 128.4 (2C), 127.2, 127.2 (2C), 114.9, 109.5, 48.7. LCMS: t_{R}=2.71; 6.42 min. [M+H]⁺=267.13. HPLC (λ₂₅₄): Purity 98.8%; HPLC (λ₂₈₀): Purity 95.6%; t_{R}: 5.55 min (method 1).

### MCK254: 1-benzyl-N³-(pyridin-3-yl)-1H-1,2,4-triazole-3,5-diamine

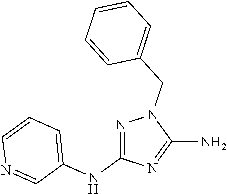 [SMILES: Nc1nc(Nc2cccnc2)nn1Cc1ccccc1]

N³-(pyridin-3-yl)-1H-1,2,4-triazole-3,5-diamine (50 mg, 0.284 mmol) was dissolved in degassed DMF, then Cs₂CO₃ (92 mg, 0.284 mmol) was added. Benzyl chloride (0.03 mL, 0.284 mmol) was added and the reaction mixture was stirred at r.t. overnight. Cs₂CO₃ was filtered and DMF was evaporated under reduced pressure. Purification by silica gel flash chromatography (CH₂Cl₂/MeOH, 10/0 then 9/1) afforded the titled compound as a brown solid (12.5 mg, 0.047 mmol, 17% yield). Brown solid, 12.5 mg, 0.047 mmol, 17% yield. Rf (CH₂Cl₂/MeOH, 9/1, v/v)=0.12. M.p.=140° C. ¹H NMR (400 MHz, DMSO-d₆) δ 8.97 (s, 1H, NH), 8.64 (d, J=2.3 Hz, 1H, CH_{Ar}), 7.93 (dd, J=12.0, 3.5 Hz, 2H, CH_{Ar}), 7.35 (t, J=7.3 Hz, 2H, CH_{Ar}), 7.30-7.24 (m, 3H, CH_{Ar}), 7.18 (dd, J=8.3, 4.6 Hz, 1H), CH_{Ar}, 6.37 (s, 2H, NH₂), 5.04 (s, 2H, CH₂). ¹³C NMR (101 MHz, DMSO-d₆) δ 156.5, 154.1, 139.2, 138.7, 137.7, 137.3, 128.2 (2C), 127.1 (2C), 127.0, 123.1, 121.3, 48.5. LCMS: t_{R}=2.69; 4.92 min. [M+H]⁺=267.20. HPLC (λ₂₅₄): Purity 97.6%; HPLC (λ₂₈₀): Purity 97.1%; t_{R}: 5.21 min (method 1).

### MCK257:1-(2,6-difluorobenzyl)-N⁵-phenyl-1H-1,2,4-triazole-3,5-diamine

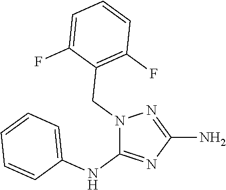 [SMILES: Nc1nc(Nc2ccccc2)n(Cc2c(F)cccc2F)n1]

N³-phenyl-1H-1,2,4-triazole-3,5-diamine (500 mg, 2.86 mmol) was dissolved in degassed DMF, then Cs₂CO₃ (930 mg, 2.86 mmol) was added. 2,6-Difluorobenzyl chloride (463 mg, 2.86 mmol) was added and the reaction mixture was stirred at r.t. overnight. Cs₂CO₃ was filtered and DMF was evaporated under reduced pressure. Purification by silica gel flash chromatography (CH₂Cl₂/THF, 10/0 then 6/4) afforded the titled compound as a white solid (46.9 mg, 0.155 mmol, 5% yield) along with the N₃-benzylated regioisomer (257 mg, 0.851 mmol, 30% yield). White solid, 46.9 mg, 0.155 mmol, 5% yield. Rf (CH₂Cl₂/THF, 1/1, v/v)=0.65. M.p.=240-242° C. ¹H NMR (400 MHz, DMSO-d₆) δ 8.86 (s, 1H, NH), 7.59 (d, J=7.7 Hz, 2H, CH_{Ar}), 7.42 (ddd, J=15.0, 8.4, 6.7 Hz, 1H, CH_{Ar}), 7.29-7.22 (m, 2H, CH_{Ar}), 7.09 (t, J=8.0 Hz, 2H, CH_{Ar}), 6.92-6.84 (m, 1H, CH_{Ar}), 5.18 (s, 2H, NH₂), 5.11 (s, 2H, CH₂). ¹³C NMR (101 MHz, DMSO-d₆) δ 160.9 (dd, J=248.3, 8.0 Hz, 2C), 160.7, 150.0, 141.1, 130.48 (t, J=10.3 Hz), 128.6 (2C), 120.4, 116.8 (2C), 112.64 (t, J=19.1 Hz), 111.6 (dd, J=18.8, 6.2 Hz, 2C), 37.21 (t, J=3.4 Hz). ¹⁹F NMR (376 MHz, DMSO-d₆) δ −114.99 (2F). LCMS: t_{R}=9.77 min. [M+H]⁺=302.07. HPLC (λ₂₅₄): Purity 95.3%; HPLC (λ₂₈₀): Purity 97.5%; t_{R}: 7.68 min (method 1).

### MCK259: 1-(2,6-difluorobenzyl)-N³-(pyridin-2-yl)-1H-1,2,4-triazole-3,5-diamine

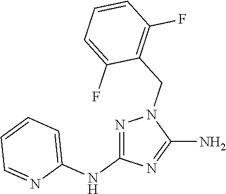 [SMILES: Nc1nc(Nc2ccccn2)nn1Cc1c(F)cccc1F]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 2,6-Difluorobenzyl chloride (230 mg, 1.42 mmol), dissolved in degassed DMF (5 mL), was added dropwise and the reaction mixture was stirred at r.t. for 1 h 30. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a white solid (87.8 mg, 0.291 mmol, 20% yield). White solid, 87.8 mg, 0.291 mmol, 20% yield. Rf (CH₂Cl₂/MeOH:9/1, v/v)=0.56. M.p.>260° C. ¹H NMR (400 MHz, DMSO-d₆) δ 9.69 (s, 1H, NH), 8.16-8.08 (m, 1H, CH_{Ar}), 7.73 (d, J=8.4 Hz, 1H, CH_{Ar}), 7.57 (ddd, J=8.8, 7.2, 2.0 Hz, 1H, CH_{Ar}), 7.43 (ddd, J=15.0, 8.5, 6.6 Hz, 1H, CH_{Ar}), 7.12 (t, J=7.9 Hz, 2H, CH_{Ar}), 6.74 (ddd, J=7.2, 4.9, 1.0 Hz, 1H, CH_{Ar}), 6.56 (s, 2H, NH₂), 5.10 (s, 2H, CH₂). ¹³C NMR (101 MHz, DMSO-d₆) δ 160.9 (dd, J=248.9, 7.9 Hz, 2C), 155.7, 154.2, 154.2, 147.5, 137.5, 130.5 (t, J=10.4 Hz), 114.8, 112.3 (t, J=19.3 Hz), 111.6 (dd, J=18.8, 6.1 Hz, 2C), 109.3, 37.44-37.1 (m). ¹⁹F NMR (376 MHz, DMSO-d₆) δ −114.67 (2F). LCMS: t_{R}=2.48; 3.80 min. [M+H]⁺=303.07. HPLC (λ₂₅₄): Purity 99.8%; HPLC (λ₂₈₀): Purity 95.3%; t_{R}: 5.53 min (method 1).

### MCK260: 1-(3-methoxybenzyl)-N⁵-phenyl-1H-1,2,4-triazole-3,5-diamine

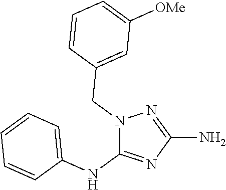 [SMILES: COc1cccc(Cn2nc(N)nc2Nc2ccccc2)c1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 3-Methoxybenzyl chloride (0.22 mL, 1.43 mmol), dissolved in degassed DMF (5 mL), was added dropwise and the reaction mixture was stirred at 0° C. for 30 min, then at r.t. for 4 h. Water was added to the mixture reaction and was extracted with ethyl acetate, washed with water, dried over magnesium sulfate and the solvents were evaporated under reduced pressure. Purification by silica gel flash chromatography (CH₂Cl₂/THF, 10/0 then 40/60) afforded the titled compound as a white solid (3 mg, 0.0102 mmol, 0.7% yield) along with the N₃-benzylated regioisomer. White solid, 3 mg, 0.0102 mmol, 0.7% yield. Rf (CH₂Cl₂/THF, 1/1, v/v)=0.74. M.p.=130-132° C. ¹H NMR (400 MHz, DMSO-d₆) δ 8.78 (s, 1H, NH), 7.58 (d, J=7.7 Hz, 2H, CH_{Ar}), 7.24 (t, J=7.6 Hz, 3H, CH_{Ar}), 6.87 (t, J=7.3 Hz, 1H, CH_{Ar}), 6.83 (dd, J=8.2, 1.8 Hz, 1H, CH_{Ar}), 6.77-6.71 (m, 2H, CH_{Ar}), 5.11 (s, 2H, NH₂), 5.09 (s, 2H, CH₂), 3.70 (s, 3H, OCH₃). ¹³C NMR (101 MHz, DMSO-d₆) δ 160.6, 159.2, 150.2, 141.1, 139.1, 129.5, 128.6 (2C), 120.4, 119.2, 116.8 (2C), 113.0, 112.2, 55.0, 48.6. LCMS: t_{R}=9.82 min. [M+H]⁺=296.07. HPLC (λ₂₅₄): Purity 90.7%; HPLC (λ₂₈₀): Purity 92.4%; t_{R}: 7.75 min (method 2).

### MCK261: 1-(3-methoxybenzyl)-N³-phenyl-1H-1,2,4-triazole-3,5-diamine

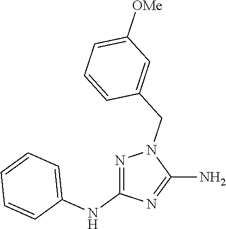 [SMILES: COc1cccc(Cn2nc(Nc3ccccc3)nc2N)c1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 3-Methoxybenzyl chloride (0.22 mL, 1.43 mmol), dissolved in degassed DMF (5 mL), was added dropwise and the reaction mixture was stirred at 0° C. for 30 min, then at r.t. for 4 h. Water was added to the mixture reaction and was extracted with ethyl acetate, washed with water, dried over magnesium sulfate and the solvents were evaporated under reduced pressure. Purification by silica gel flash chromatography (CH₂Cl₂/THF, 10/0 then 40/60) afforded the titled compound as a white solid (33.7 mg, 0.114 mmol, 8% yield) along with the N₅-benzylated regioisomer. White solid, 33.7 mg, 0.114 mmol, 8% yield. Rf (CH₂Cl₂/THF, 1/1, v/v)=0.69. M.p.=106-108° C. ¹H NMR (400 MHz, DMSO-d₆) δ 8.69-8.64 (m, 1H, NH), 7.46 (d, J=7.7 Hz, 2H, CH_{Ar}), 7.25 (td, J=7.5, 1.3 Hz, 1H, CH_{Ar}), 7.14 (t, J=7.9 Hz, 2H, CH_{Ar}), 6.86-6.78 (m, 3H, CH_{Ar}), 6.70 (t, J=7.3 Hz, 1H, CH_{Ar}), 6.27 (s, 2H, NH₂), 5.03-4.94 (m, 2H, CH₂), 3.72 (s, 3H, CH₃). ¹³C NMR (101 MHz, DMSO-d₆) δ 159.2, 157.1, 154.1, 142.3, 139.1, 129.4, 128.4 (2C), 119.4, 118.3, 115.5 (2C), 113.2, 112.3, 54.9, 48.5. LCMS: t_{R}=9.71 min. [M+H]⁺=296.00. HPLC (λ₂₅₄): Purity 96.7%; t_{R}: 8.18 min (method 2).

### MCK262: 1-(4-isopropylbenzyl)-N⁵-phenyl-1H-1,2,4-triazole-3,5-diamine

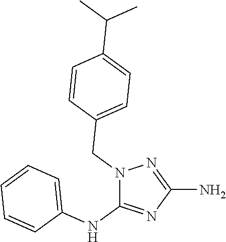 [SMILES: CC(C)c1ccc(Cn2nc(N)nc2Nc2ccccc2)cc1]

N³-phenyl-1H-1,2,4-triazole-3,5-diamine (250 mg, 1.43 mmol) was dissolved in anhydrous DMF (1 mL), then potassium tert-butoxide (160 mg, 1.43 mmol) was added. 4-Isopropylbenzyl chloride (0.22 mL, 1.43 mmol), dissolved in anhydrous CH₂Cl₂ (1 mL), was added dropwise and the reaction mixture was stirred at 0° C. for 1 h and at r.t. for 2 h. Water was added to the mixture reaction and the solvents were evaporated under reduced pressure. Purification by reversed-phase chromatography (H₂O/CH₃CN, 10/0 then 45/55) afforded the titled compound as a white solid (51.8 mg, 0.169 mmol, 12% yield) along with the N₃-benzylated regioisomer. White solid, 51.8 mg, 0.169 mmol, 12% yield). Rf (CH₂Cl₂/THF, 1/1, v/v)=0.81. M.p.=146° C. ¹H NMR (400 MHz, DMSO-d₆) δ 8.78 (s, 1H, NH), 7.59 (d, J=7.7 Hz, 2H, CH_{Ar}), 7.27-7.21 (m, 2H, CH_{Ar}), 7.19 (d, J=8.1 Hz, 2H, CH_{Ar}), 7.11 (d, J=8.1 Hz, 2H, CH_{Ar}), 6.87 (t, J=7.3 Hz, 1H, CH_{Ar}), 5.09 (s, 2H, NH₂), 5.07 (s, 2H, CH₂), 2.84 (p, J=6.9 Hz, 1H, CH), 1.16 (d, J=6.9 Hz, 6H, CH₃). ¹³C NMR (101 MHz, DMSO-d₆) δ 160.6, 150.0, 147.4, 141.1, 134.9, 128.6 (2C), 127.2 (2C), 126.2 (2C), 120.4, 116.8 (2C), 48.4, 33.1, 23.8 (2C). LCMS: t_{R}=11.15 min. [M+H]⁺=308.07. HPLC (λ₂₅₄): Purity 91.1%; t_{R}: 9.47 min (method 2).

### MCK263: 1-(4-isopropylbenzyl)-N³-phenyl-1H-1,2,4-triazole-3,5-diamine

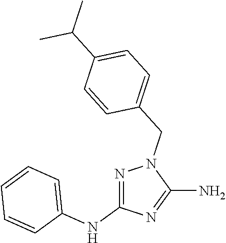 [SMILES: CC(C)c1ccc(Cn2nc(Nc3ccccc3)nc2N)cc1]

N³-phenyl-1H-1,2,4-triazole-3,5-diamine (250 mg, 1.43 mmol) was dissolved in anhydrous DMF (1 mL), then potassium tert-butoxide (160 mg, 1.43 mmol) was added. 4-Isopropylbenzyl chloride (0.22 mL, 1.43 mmol), dissolved in anhydrous CH₂Cl₂ (1 mL), was added dropwise and the reaction mixture was stirred at 0° C. for 1 h and at r.t. for 2 h. Water was added to the mixture reaction and the solvents were evaporated under reduced pressure. Purification by reversed-phase chromatography (H₂O/CH₃CN, 10/0 then 45/55) afforded the titled compound as a white solid (122 mg, 0.397 mmol, 28% yield) along with the N₅-benzylated regioisomer. White solid, 122 mg, 0.397 mmol, 28% yield. Rf (CH₂Cl₂/THF, 1/1, v/v)=0.79. M.p.=140-142° C. ¹H NMR (400 MHz, DMSO-d₆) δ 8.66 (s, 1H, NH), 7.47 (d, J=7.8 Hz, 2H, CH_{Ar}), 7.17 (dq, J=15.6, 8.2 Hz, 6H, CH_{Ar}), 6.70 (t, J=7.3 Hz, 1H, CH_{Ar}), 6.27 (s, 2H, NH₂), 4.98 (s, 2H, CH₂), 2.84 (hept, J=6.9 Hz, 1H, CH), 1.16 (s, 6H, CH₃). ¹³C NMR (101 MHz, DMSO-d₆) δ 157.0, 154.0, 147.3, 142.3, 135.0, 128.4 (2C), 127.3 (2C), 126.2 (2C), 118.3, 115.5 (2C), 48.4, 33.1, 23.9 (2C). LCMS: t_{R}=11.15 min. [M+H]⁺=308.07. HPLC (λ₂₅₄): Purity 97.3%; t_{R}: 9.82 min (method 2).

### MCK264: 1-(3-bromobenzyl)-N³-phenyl-1H-1,2,4-triazole-3,5-diamine

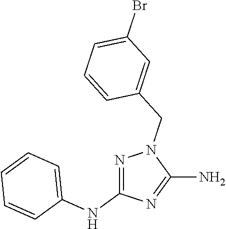 [SMILES: Nc1nc(Nc2ccccc2)nn1Cc1cccc(Br)c1]

N³-phenyl-1H-1,2,4-triazole-3,5-diamine (150 mg, 0.857 mmol) was dissolved in anhydrous DMF (1 mL), then potassium tert-butoxide (96 mg, 0.857 mmol) was added. 3-Bromobenzyl bromide (225 mg, 0.857 mmol), dissolved in anhydrous CH₂Cl₂ (1 mL), was added dropwise and the reaction mixture was stirred at 0° C. for 1 h and at r.t. for an additional 1 h. Water was added to the mixture reaction and the solvents were evaporated under reduced pressure. Purification by reversed-phase chromatography (H₂O/CH₃CN, 10/0 then 45/55) afforded the titled compound as a purple solid (89 mg, 0.259 mmol, 30% yield) along with the N₅-benzylated regioisomer. Purple solid, 89 mg, 0.259 mmol, 30% yield. Rf (CH₂Cl₂/THF, 1/1, v/v)=0.56. M.p.=108-110° C. ¹H NMR (400 MHz, DMSO-d₆) δ 8.69 (s, 1H, NH), 7.46 (q, J=6.2, 5.5 Hz, 4H, CH_{Ar}), 7.32 (t, J=7.8 Hz, 1H, CH_{Ar}), 7.25 (d, J=7.7 Hz, 1H, CH_{Ar}), 7.14 (t, J=7.7 Hz, 2H, CH_{Ar}), 6.71 (t, J=7.3 Hz, 1H, CH_{Ar}), 6.34 (s, 2H, NH₂), 5.04 (s, 2H, CH₂). ¹³C NMR (101 MHz, DMSO-d₆) δ 157.2, 154.2, 142.2, 140.3, 130.6, 130.0, 128.4 (2C), 126.3, 121.6, 118.4, 115.5 (2C), 48.0. LCMS: t_{R}=10.75 min. [M+H]⁺=244.00; 246.00. HPLC (λ₂₅₄): Purity 99.4%; HPLC (λ₂₈₀): Purity 96.7%; t_{R}: 8.85 min (method 2).

### MCK268: 1-(2,6-dichlorobenzyl)-N³-phenyl-1H-1,2,4-triazole-3,5-diamine

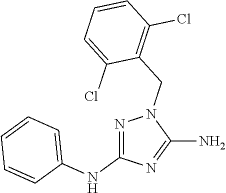 [SMILES: Nc1nc(Nc2ccccc2)nn1Cc1c(Cl)cccc1Cl]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 2,6-Dichlorobenzyl chloride (276 mg, 1.43 mmol), dissolved in degassed DMF (5 mL), was added dropwise and the reaction mixture was stirred at 0° C. for 30 min, then at r.t. for 3 h. Water was added to the mixture reaction and was extracted with ethyl acetate, washed with water, dried over magnesium sulfate and the solvents were evaporated under reduced pressure. Purification by silica gel flash chromatography (CH₂Cl₂/THF, 10/0 then 50/50) afforded the titled compound as a brown solid (93.3 mg, 0.028 mmol, 20% yield). Brown solid, 93.3 mg, 0.028 mmol, 20% yield. Rf (CH₂Cl₂/THF, 8/2, v/v)=0.57. M.p.=206-208° C. ¹H NMR (400 MHz, DMSO-d₆) δ 8.61 (s, 1H, NH), 7.51 (d, J=8.0 Hz, 2H, CH_{Ar}), 7.43-7.33 (m, 3H, CH_{Ar}), 7.07 (t, J=7.9 Hz, 2H, CH_{Ar}), 6.66 (t, J=7.3 Hz, 1H, CH_{Ar}), 6.37 (s, 2H, NH₂), 5.16 (s, 2H, CH₂). ¹³C NMR (101 MHz, DMSO-d₆) δ 157.1, 154.0, 142.4, 135.6 (2C), 131.7, 130.4, 128.5 (2C), 128.2 (2C), 118.2, 115.4 (2C), 44.6. LCMS: t_{R}=10.31 min. [M+H]⁺=334.00. HPLC (λ₂₅₄): Purity 100%; HPLC (λ₂₈₀): Purity 96.9%; t_{R}: 8.78 min (method 2).

### MCK269: 1-(2-methylbenzyl)-N³-(pyridin-2-yl)-1H-1,2,4-triazole-3,5-diamine

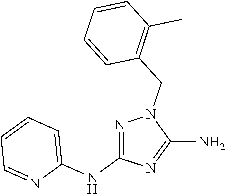 [SMILES: Cc1ccccc1Cn1nc(Nc2ccccn2)nc1N]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 2-Methylbenzyl chloride (0.19 mL, 1.42 mmol), dissolved in degassed DMF (5 mL), was added dropwise and the reaction mixture was stirred at r.t. for 3 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (210.9 mg, 0.753 mmol, 53% yield). Yellow solid, 210.9 mg, 0.753 mmol, 53% yield. Rf (CH₂Cl₂/MeOH:95/5, v/v)=0.44. M.p.=256-258° C. ¹H NMR (400 MHz, DMSO-d₆) δ 10.09 (s, 1H, NH), 8.17 (d, J=3.8 Hz, 1H, CH_{Ar}), 7.84 (d, J=8.5 Hz, 1H, CH_{Ar}), 7.71-7.55 (m, 1H, CH_{Ar}), 7.16 (dq, J=12.8, 7.0 Hz, 3H, CH_{Ar}), 6.90-6.75 (m, 2H, CH_{Ar}), 6.56 (s, 2H, NH₂), 5.07 (s, 2H, CH₂), 2.35 (s, 3H, CH₃). ¹³C NMR (101 MHz, DMSO-d₆) δ 155.6, 154.5, 154.2, 147.6, 137.7, 135.5, 135.3, 129.9, 127.0, 126.4, 125.8, 114.9, 109.5, 47.0, 18.6. LCMS: t_{R}=2.53; 3.64 min. [M+H]⁺=281.07. HPLC (λ₂₅₄): Purity 99.9%; HPLC (λ₂₈₀): Purity 97.4%; t_{R}: 5.85 min (method 1).

### MCK271: 1-(4-methylbenzyl)-N³-phenyl-1H-1,2,4-triazole-3,5-diamine

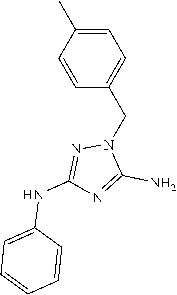 [SMILES: Cc1ccc(Cn2nc(Nc3ccccc3)nc2N)cc1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 4-Methylbenzyl chloride (0.19 mL, 1.43 mmol), dissolved in degassed DMF (5 mL), was added dropwise and the reaction mixture was stirred at 0° C. for 30 min, then at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/THF, 10/0 then 50/50) afforded the titled compound as a purple solid (28 mg, 0.100 mmol, 7% yield). Purple solid, 28 mg, 0.100 mmol, 7% yield. Rf (CH₂Cl₂/THF, 1/1, v/v)=0.56. M.p.=116-118° C. ¹H NMR (400 MHz, DMSO-d₆) δ 8.70 (s, 11H, NH), 7.45 (d, J=7.7 Hz, 2H, CH_{Ar}), 7.14 (s, 6H, CH_{Ar}), 6.72 (t, J=7.3 Hz, 1H, CH_{Ar}), 6.37 (s, 2H, NH₂), 4.97 (s, 2H, CH₂), 2.26 (s, 3H, CH₃). ¹³C NMR (101 MHz, DMSO-d₆) δ 156.3, 153.5, 142.1, 136.3, 134.4, 128.8 (2C), 128.4 (2C), 127.2 (2C), 118.5, 115.5 (2C), 48.5, 20.6. LCMS: t_{R}=9.82 min. [M+H]⁺=280.00. HPLC (λ₂₅₄): Purity 99.7%; HPLC (so): Purity 95.0%; t_{R}: 8.61 min (method 2).

### MCK272: 1-(4-chlorobenzyl)-N³-phenyl-1H-1,2,4-triazole-3,5-diamine

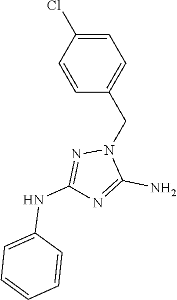 [SMILES: Nc1nc(Nc2ccccc2)nn1Cc1ccc(Cl)cc1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 4-Chlorobenzyl chloride (230 mg, 1.43 mmol), dissolved in degassed DMF (5 mL), was added dropwise and the reaction mixture was stirred at 0° C. for 30 min, then at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/THF, 10/0 then 40/60) afforded the titled compound as a purple solid (76.9 mg, 0.257 mmol, 18% yield).

Purple solid, 76.9 mg, 0.257 mmol, 18% yield. Rf (CH₂Cl₂/THF, 1/1, v/v)=0.47. M.p.=156-158° C. ¹H NMR (400 MHz, DMSO-d₆) δ 8.69 (s, 1H, NH), 7.43 (dd, J=14.6, 8.1 Hz, 4H, CH_{Ar}), 7.26 (d, J=8.1 Hz, 2H, CH_{Ar}), 7.14 (t, J=7.8 Hz, 2H, CH_{Ar}), 6.71 (t, J=7.3 Hz, 1H, CH_{Ar}), 6.34 (s, 2H, NH₂), 5.03 (s, 2H, CH₂). ¹³C NMR (101 MHz, DMSO-d₆) δ 157.0, 154.0, 142.2, 136.5, 131.8, 129.1 (2C), 128.4 (2C), 128.3 (2C), 118.4, 115.5 (2C), 48.0. LCMS: t_{R}=10.63 min. [M+H]⁺=300.07. HPLC (λ₂₅₄): Purity 99.7%; HPLC (λ₂₈₀): Purity 98.5%; t_{R}: 8.98 min (method 2).

### MCK273: N-phenyl-1-(3-(trifluoromethyl)benzyl)-1H-1,2,4-triazole-3,5-diamine

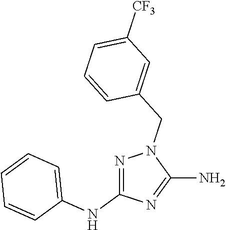 [SMILES: Nc1nc(Nc2ccccc2)nn1Cc1cccc(C(F)(F)F)c1]

N³-phenyl-1H-1,2,4-triazole-3,5-diamine (250 mg, 1.43 mmol) was dissolved in anhydrous DMF (1 mL), then potassium tert-butoxide (160 mg, 1.43 mmol) was added. 3-(Trifluoromethyl)benzyl chloride (0.22 mL, 1.43 mmol), dissolved in anhydrous CH₂Cl₂ (1 mL), was added dropwise and the reaction mixture was stirred at 0° C. for 1 h and at r.t. for 2 h. Water was added to the mixture reaction and the solvents were evaporated under reduced pressure. Purification by reversed-phase chromatography (H₂O/CH₃CN, 10/0 then 45/55) afforded the titled compound as a yellow solid (197.1 mg, 0.592 mmol, 41% yield) along with the N₅-benzylated regioisomer. Yellow solid, 197.1 mg, 0.592 mmol, 41% yield. Rf (CH₂Cl₂/THF, 1/1, v/v)=0.71. M.p.=118-120° C. ¹H NMR (400 MHz, DMSO-d₆) δ 8.69 (s, 1H, NH), 7.68-7.57 (m, 3H, CH_{Ar}), 7.54 (d, J=7.6 Hz, 1H, CH_{Ar}), 7.45 (d, J=7.8 Hz, 2H, CH_{Ar}), 7.14 (t, J=7.9 Hz, 2H, CH_{Ar}), 6.71 (t, J=7.3 Hz, 1H, CH_{Ar}), 6.35 (s, 2H, NH₂), 5.13 (s, 2H, CH₂). ¹³C NMR (101 MHz, DMSO-d₆) δ 157.3, 154.2, 142.2, 139.0, 131.4, 129.5 (2C), 129.2, 128.9, 128.4 (2C), 124.0-123.8 (m), 118.5, 115.5 (2C), 48.1. ¹⁹F NMR (376 MHz, DMSO-d₆) δ −61.11 (3F). LCMS: t_{R}=10.58 min. [M+H]⁺=334.07. HPLC (λ₂₅₄): Purity 98.1%; t_{R}: 9.59 min (method 2).

### MCK274: 1-(3,5-difluorobenzyl)-N³-phenyl-1H-1,2,4-triazole-3,5-diamine

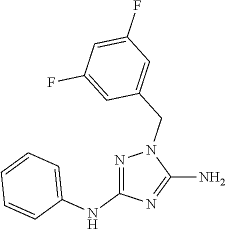 [SMILES: Nc1nc(Nc2ccccc2)nn1Cc1cc(F)cc(F)c1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 3,5-Difluorobenzyl chloride (230 mg, 1.43 mmol), dissolved in degassed DMF (5 mL), was added dropwise and the reaction mixture was stirred at 0° C. for 30 min, then at r.t. for 3 h. Water was added to the mixture reaction and was extracted with ethyl acetate, washed with water, dried over magnesium sulfate and the solvents were evaporated under reduced pressure. Purification by silica gel flash chromatography (CH₂Cl₂/THF, 10/0 then 50/50) afforded the titled compound as a purple solid (20.1 mg, 0.066 mmol, 5% yield).

Purple solid, 20.1 mg, 0.066 mmol, 5% yield. Rf (CH₂Cl₂/THF, 1/1, v/v)=0.61. M.p.=146-148° C. ¹H NMR (400 MHz, DMSO-d₆) δ 8.82 (s, 1H, NH), 7.46 (d, J=8.0 Hz, 2H, CH_{Ar}), 7.16 (t, J=7.9 Hz, 3H, CH_{Ar}), 6.94 (d, J=6.5 Hz, 2H, CH_{Ar}), 6.74 (t, J=7.3 Hz, 1H, CH_{Ar}), 6.54 (s, 2H, NH₂), 5.09 (s, 2H, CH₂). ¹³C NMR (101 MHz, DMSO-d₆) δ 162.36 (dd, J=246.4, 13.1 Hz, 2C), 156.4, 153.8, 142.0, 141.96 (t, J=4.4 Hz), 128.5 (2C), 118.8, 115.7 (2C), 110.3 (dd, J=18.7, 6.9 Hz, 2C), 102.79 (t, J=25.7 Hz), 47.9. ¹⁹F NMR (376 MHz, DMSO-d₆) δ −109.66 (2F). LCMS: t_{R}=10.13 min. [M+H]⁺=302.07. HPLC (λ₂₅₄): Purity 98.4%; ta: 8.88 min (method 2).

### MCK275: 1-(3-nitrobenzyl))-N³-phenyl-1H-1,2,4-triazole-3,5-diamine

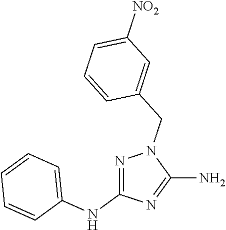 [SMILES: Nc1nc(Nc2ccccc2)nn1Cc1cccc([N+](=O)[O-])c1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 3-Nitrobenzyl bromide (307 mg, 1.43 mmol), dissolved in degassed DMF (5 mL), was added dropwise and the reaction mixture was stirred at 0° C. for 30 min, then at r.t. for 3 h. Water was added to the mixture reaction and was extracted with ethyl acetate, washed with water, dried over magnesium sulfate and the solvents were evaporated under reduced pressure. Purification by silica gel flash chromatography (CH₂Cl₂/THF, 10/0 then 50/50) afforded the titled compound as a yellow solid (37.3 mg, 0.120 mmol, 8% yield). Yellow solid, 37.3 mg, 0.120 mmol, 8% yield. Rf (CH₂Cl₂/THF, 1/1, v/v)=0.51. M.p.=180-182° C. ¹H NMR (400 MHz, DMSO-d₆) δ 9.12 (s, 1H, NH), 8.24-8.12 (m, 2H, CH_{Ar}), 7.76 (d, J=7.7 Hz, 1H, CH_{Ar}), 7.69 (t, J=7.8 Hz, 1H, CH_{Ar}), 7.43 (d, J=7.7 Hz, 2H, CH_{Ar}), 7.26-7.15 (m, 2H, CH_{Ar}), 6.82 (t, J=7.3 Hz, 1H, CH_{Ar}), 5.25 (s, 2H, CH₂). ¹³C NMR (101 MHz, DMSO-d₆) δ 149.4, 148.9, 147.8, 139.7, 137.8, 134.3, 130.2, 128.9 (2C), 122.8, 122.5, 120.9, 116.6 (2C), 48.6. LCMS: t_{R}=9.77 min. [M+H]⁺=311.07. HPLC (λ₂₅₄): Purity 98.0%; HPLC (λ₂₈₀): Purity 95.3%; t_{R}: 8.61 min (method 2).

### MCK276: 1-(3-bromobenzyl)-N³-(pyridin-2-yl)-1H-1,2,4-triazole-3,5-diamine

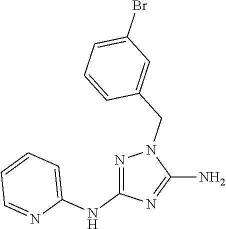 [SMILES: Nc1nc(Nc2ccccn2)nn1Cc1cccc(Br)c1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 3-Bromobenzyl bromide (352 mg, 1.42 mmol), dissolved in degassed DMF (5 mL), was added dropwise and the reaction mixture was stirred at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (121.9 mg, 0.352 mmol, 25% yield). Yellow solid, 121.9 mg, 0.352 mmol, 25% yield. Rf (CH₂Cl₂/MeOH:9/1, v/v)=0.50. M.p.=232-234° C. ¹H NMR (400 MHz, DMSO-d₆) δ 9.97 (s, 1H, NH), 8.18 (ddd, J=4.9, 2.0, 0.9 Hz, 1H, CH_{Ar}), 7.84 (dt, J=8.6, 1.0 Hz, 1H, CH_{Ar}), 7.63 (ddd, J=8.8, 7.2, 2.0 Hz, 1H, CH_{Ar}), 7.54-7.43 (m, 2H, CH_{Ar}), 7.33 (t, J=7.8 Hz, 1H, CH_{Ar}), 7.25 (dt, J=7.8, 1.3 Hz, 1H, CH_{Ar}), 6.79 (ddd, J=7.2, 4.9, 1.1 Hz, 1H, CH_{Ar}), 6.62 (s, 2H, NH₂), 5.10 (s, 2H, CH₂). ¹³C NMR (101 MHz, DMSO-d₆) δ 155.8, 154.4, 154.1, 147.6, 140.1, 137.8, 130.7, 130.1, 129.9, 126.3, 121.6, 115.0, 109.5, 48.0. LCMS: t_{R}=7.40 min. [M+H]⁺=345.07; 347.07. HPLC (λ₂₅₄): Purity 99.4%; t_{R}: 6.55 min (method 1).

### MCK278: 1-(2,6-dichlorobenzyl)-N³-(pyridin-2-yl)-1H-1,2,4-triazole-3,5-diamine

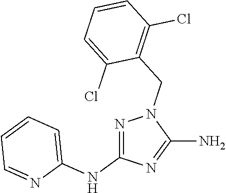 [SMILES: Nc1nc(Nc2ccccn2)nn1Cc1c(Cl)cccc1Cl]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 2,6-Dichlorobenzyl chloride (274 mg, 1.42 mmol), dissolved in degassed DMF (5 mL), was added dropwise and the reaction mixture was stirred at r.t. for 3 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a white solid (30.9 mg, 0.093 mmol, 7% yield). White solid, 30.9 mg, 0.093 mmol, 7% yield. Rf (CH₂Cl₂/MeOH:9/1, v/v)=0.42. M.p.>60° C. ¹H NMR (400 MHz, DMSO-d₆) δ 9.57 (s, 1H, NH), 8.11 (dd, J=5.0, 1.8 Hz, 1H, CH_{Ar}), 7.72-7.66 (m, 1H, CH_{Ar}), 7.52 (dq, J=5.7, 2.9, 1.9 Hz, 3H, CH_{Ar}), 7.39 (dd, J=8.7, 7.4 Hz, 1H, CH_{Ar}), 6.72 (ddd, J=7.2, 4.8, 1.0 Hz, 1H, CH_{Ar}), 6.57 (s, 2H, NH₂), 5.20 (s, 2H, CH₂). ¹³C NMR (101 MHz, DMSO-d₆) δ 155.6, 154.2, 154.2, 147.5, 137.4, 135.6 (2C), 131.5, 130.5, 128.6 (2C), 114.8, 109.3, 44.7. LCMS: t_{R}=2.57; 2.08 min. [M+H]⁺=335.00. HPLC (λ₂₅₄): Purity 97.2%; t_{R}: 6.26 min (method 1).

### MCK279: 1-(4-methylbenzyl)-N³-(pyridin-2-yl)-1H-1,2,4-triazole-3,5-diamine

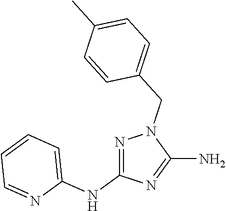 [SMILES: Cc1ccc(Cn2nc(Nc3ccccn3)nc2N)cc1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 4-Methylbenzyl chloride (0.19 mL, 1.42 mmol), dissolved in degassed DMF (5 mL), was added dropwise and the reaction mixture was stirred at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (99.9 mg, 0.357 mmol, 25% yield). Yellow solid, 99.9 mg, 0.357 mmol, 25% yield. Rf (CH₂Cl₂/MeOH:9/1, v/v)=0.52. M.p.=210-212° C. ¹H NMR (400 MHz, DMSO-d₆) δ 10.03 (s, 1H, NH), 8.23-8.13 (m, 1H, CH_{Ar}), 7.85 (d, J=8.5 Hz, 1H, CH_{Ar}), 7.63 (td, J=8.8, 8.1, 1.9 Hz, 1H, CH_{Ar}), 7.15 (s, 4H, CH_{Ar}), 6.78 (ddd, J=7.2, 4.9, 1.1 Hz, 1H, CH_{Ar}), 6.56 (s, 2H, NH₂), 5.03 (s, 2H, CH₂), 2.27 (s, 3H, CH₃). ¹³C NMR (101 MHz, DMSO-d₆) δ 155.5, 154.2, 154.2, 147.5, 137.8, 136.3, 134.3, 128.9 (2C), 127.2 (2C), 114.9, 109.5, 48.5, 20.6. LCMS: t_{R}=2.45; 3.95 min. [M+H]⁺=281.07. HPLC (λ₂₅₄): Purity 99.5%; HPLC (λ₂₈₀): Purity 97.1%; t_{R}: 6.13 min (method 1).

### MCK280: 1-(4-chlorobenzyl)-N³-(pyridin-2-yl)-1H-1,2,4-triazole-3,5-diamine

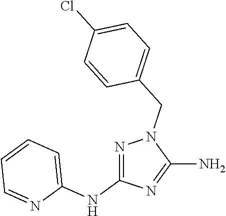 [SMILES: Nc1nc(Nc2ccccn2)nn1Cc1ccc(Cl)cc1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 4-Chlorobenzyl chloride (229 mg, 1.42 mmol), dissolved in degassed DMF (5 mL), was added dropwise and the reaction mixture was stirred at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (70.9 mg, 0.236 mmol, 17% yield). Yellow solid, 70.9 mg, 0.236 mmol, 17% yield. Rf (CH₂Cl₂/MeOH, 9/1, v/v)=0.47. M.p.=230-232° C. ¹H NMR (400 MHz, DMSO-d₆) δ 9.72 (s, 1H, NH), 8.15 (ddd, J=4.9, 1.9, 0.9 Hz, 1H, CH_{Ar}), 7.83 (d, J=8.5 Hz, 1H, CH_{Ar}), 7.62 (ddd, J=8.8, 7.2, 2.0 Hz, 1H, CH_{Ar}), 7.49-7.36 (m, 2H, CH_{Ar}), 7.26 (d, J=8.5 Hz, 2H, CH_{Ar}), 6.77 (ddd, J=7.2, 4.9, 1.0 Hz, 1H, CH_{Ar}), 6.54 (s, 2H, NH₂), 5.07 (s, 2H, CH₂). ¹³C NMR (101 MHz, DMSO-d₆) δ 155.8, 154.4, 154.1, 147.6, 137.7, 136.4, 131.8, 129.1 (2C), 128.4 (2C), 114.9, 109.5, 48.0. LCMS: t_{R}=2.50; 3.49 min. [M+H]⁺=301.13. HPLC (λ₂₅₄): Purity 98.4%; t_{R}: 6.47 min (method 1).

### MCK281: 4-((5-amino-3-(pyridin-2-ylamino)-1H-1,2,4-triazol-1-yl)methyl)benzonitrile

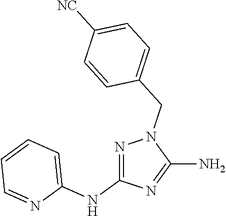 [SMILES: N#Cc1ccc(Cn2nc(Nc3ccccn3)nc2N)cc1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 4-(Chloromethyl)benzonitrile (220 mg, 1.42 mmol), dissolved in degassed DMF (5 mL), was added dropwise and the reaction mixture was stirred at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (49.8 mg, 0.171 mmol, 12% yield). Yellow solid, 49.8 mg, 0.171 mmol, 12% yield. Rf (CH₂Cl₂/MeOH:9/1, v/v)=0.47. M.p.=242° C. ¹H NMR (400 MHz, DMSO-d₆) δ 10.07 (s, 1H, NH), 8.20-8.15 (m, 1H, CH_{Ar}), 7.84 (d, J=8.2 Hz, 3H, CH_{Ar}), 7.63 (ddd, J=8.8, 7.2, 2.0 Hz, 1H, CH_{Ar}), 7.40 (d, J=8.3 Hz, 2H, CH_{Ar}), 6.79 (ddd, J=7.2, 4.9, 1.0 Hz, 1H, CH_{Ar}), 6.65 (s, 2H, NH₂), 5.21 (s, 2H, CH₂). ¹³C NMR (101 MHz, DMSO-d₆) δ 155.9, 154.6, 154.1, 147.6, 143.1, 137.8, 132.4 (2C), 127.9 (2C), 118.7, 115.0, 110.0, 109.5, 48.4. LCMS: t_{R}=2.68; 4.46 min. [M+H]⁺=292.07. HPLC (λ₂₅₄): Purity 99.8%; t_{R}: 5.26 min (method 1).

### MCK282: N³-(pyridin-2-yl)-1-(3-(trifluoromethyl)benzyl)-1H-1,2,4-triazole-3,5-diamine

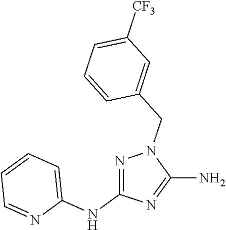 [SMILES: Nc1nc(Nc2ccccn2)nn1Cc1cccc(C(F)(F)F)c1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 3-(Trifluoromethyl)benzyl chloride (0.22 mL, 1.42 mmol), dissolved in degassed DMF (5 mL), was added dropwise and the reaction mixture was stirred at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a white solid (194.7 mg, 0.583 mmol, 41% yield). White solid, 194.7 mg, 0.583 mmol, 41% yield. Rf (CH₂Cl₂/MeOH:9/1, v/v)=0.42. M.p.=248-250° C. ¹H NMR (400 MHz, DMSO-d₆) δ 9.86 (s, 1H, NH), 8.23-8.12 (m, 1H, CH_{Ar}), 7.84 (d, J=8.5 Hz, 1H, CH_{Ar}), 7.69-7.58 (m, 4H, CH_{Ar}), 7.54 (d, J=7.8 Hz, 1H, CH_{Ar}), 6.78 (ddd, J=7.3, 4.9, 1.0 Hz, 1H, CH_{Ar}), 6.62 (s, 2H, NH₂), 5.19 (s, 2H, CH₂). ¹³C NMR (101 MHz, DMSO-d₆) δ 155.9, 154.5, 154.1, 147.6, 138.8, 137.7, 131.4, 129.6, 129.1 (q, J=31.5 Hz), 124.1 (q, J=272.2 Hz), 124.1 (q, J=3.8 Hz), 123.8 (q, J=3.9 Hz), 115.0, 109.5, 48.2. ¹⁹F NMR (376 MHz, DMSO-d₆) δ −61.12 (3F). LCMS: t_{R}=4.00 min. [M+H]⁺=335.07. HPLC (0.254): Purity 99.7%; HPLC (λ₂₈₀): Purity 99.3%; t_{R}: 6.75 min (method 1).

### MCK283: 1-(3,5-difluorobenzyl)-N-(pyridin-2-yl)-1H-1,2,4-triazole-3,5-diamine

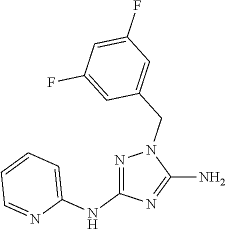 [SMILES: Nc1nc(Nc2ccccn2)nn1Cc1cc(F)cc(F)c1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 3,5-Difluorobenzyl chloride (232 mg, 1.42 mmol), dissolved in degassed DMF (5 mL), was added dropwise and the reaction mixture was stirred at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (124.8 mg, 0.413 mmol, 29% yield). Yellow solid, 124.8 mg, 0.413 mmol, 29% yield. Rf (CH₂Cl₂/MeOH:9/1, v/v)=0.45. M.p.=258° C. ¹H NMR (400 MHz, DMSO-d₆) δ 10.08 (s, 11H, NH), 8.25-8.15 (m, 1H, CH_{Ar}), 7.85 (d, J=8.5 Hz, 1H, CH_{Ar}), 7.64 (ddd, J=8.8, 7.2, 2.0 Hz, 1H, CH_{Ar}), 7.17 (tt, J=9.4, 2.4 Hz, 1H, CH_{Ar}), 6.97-6.89 (m, 2H, CH_{Ar}), 6.80 (ddd, J=7.2, 4.9, 1.0 Hz, 1H, CH_{Ar}), 6.66 (s, 2H, NH₂), 5.13 (s, 2H, CH₂). ¹³C NMR (101 MHz, DMSO-d₆) δ 162.3 (dd, J=246.4, 13.1 Hz, 2C), 155.9, 154.5, 154.1, 147.6, 141.9 (t, J=9.0 Hz), 137.88, 115.07, 110.2 (d, J=6.8, 18.7 Hz, 2C), 109.5, 102.8 (t, J=25.7 Hz), 47.9. ¹⁹F NMR (376 MHz, DMSO-d₆) δ −109.58 (2F). LCMS: t_{R}=2.66; 4.80 min. [M+H]⁺=303.13. HPLC (λ₂₅₄): Purity 98.7%; t_{R}: 6.03 min (method 1).

### MCK284: 1-(2,4-difluorobenzyl)-N³-(pyridin-2-yl)-1H-1,2,4-triazole-3,5-diamine

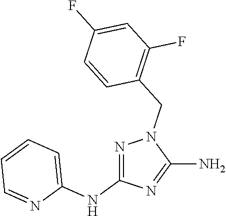 [SMILES: Nc1nc(Nc2ccccn2)nn1Cc1ccc(F)cc1F]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 2,4-Difluorobenzyl chloride (232 mg, 1.42 mmol), dissolved in degassed DMF (5 mL), was added dropwise and the reaction mixture was stirred at r.t. for 2 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (199.3 mg, 0.660 mmol, 46% yield). Yellow solid, 199.3 mg, 0.660 mmol, 46% yield. Rf (CH₂Cl₂/MeOH:9/1, v/v)=0.40. M.p.>60° C. ¹H NMR (400 MHz, DMSO-d₆) δ 9.92 (s, 1H, NH), 8.20-8.12 (m, 1H, CH_{Ar}), 7.82 (d, J=8.5 Hz, 1H, CH_{Ar}), 7.62 (ddd, J=8.8, 7.2, 2.0 Hz, 1H, CH_{Ar}), 7.27 (ddd, J=10.5, 9.3, 2.5 Hz, 1H, CH_{Ar}), 7.17 (td, J=8.6, 6.6 Hz, 1H, CH_{Ar}), 7.08 (td, J=8.6, 2.6 Hz, 1H, CH_{Ar}), 6.78 (ddd, J=7.2, 4.9, 1.0 Hz, 1H, CH_{Ar}), 6.60 (s, 2H, NH₂), 5.12 (s, 2H, CH₂). ¹³C NMR (101 MHz, DMSO-d₆) δ 161.9 (dd, J=182.2, 12.2 Hz), 159.5 (dd, J=184.6, 12.3 Hz), 155.9, 154.5, 154.1, 147.0, 137.7, 130.4 (dd, J=9.9, 5.9 Hz), 120.66 (dd, J=15.1, 3.7 Hz), 114.9, 111.5 (dd, J=21.3, 3.6 Hz), 109.5, 103.9 (t, J=25.7 Hz), 42.7 (d, J=2.8 Hz). ¹⁹F NMR (376 MHz, DMSO-d₆) δ −111.33, −113.74. LCMS: t_{R}=2.73; 6.94 min. [M+H]⁺=303.13. HPLC (λ₂₅₄): Purity 97.1%; t_{R}: 5.60 min (method 1).

### MCK288: 1-(4-nitrobenzyl))-N³-phenyl-1H-1,2,4-triazole-3,5-diamine

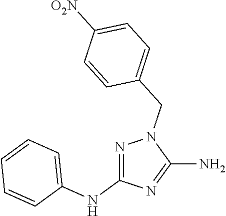 [SMILES: Nc1nc(Nc2ccccc2)nn1Cc1ccc([N+](=O)[O-])cc1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 4-Nitrobenzyl bromide (307 mg, 1.43 mmol), dissolved in degassed DMF (5 mL), was added dropwise and the reaction mixture was stirred at 0° C. for 30 min, then at r.t. for 3 h. Water was added to the mixture reaction and was extracted with ethyl acetate, washed with water, dried over magnesium sulfate and the solvents were evaporated under reduced pressure. Purification by silica gel flash chromatography (CH₂Cl₂/THF, 10/0 then 50/50) and second purification by silica gel flash chromatography (CH₂Cl₂/THF, iso 50/50) afforded the titled compound as a yellow solid (21.1 mg, 0.068 mmol, 5% yield). Yellow solid, 21.1 mg, 0.068 mmol, 5% yield. Rf (CH₂Cl₂/THF, 1/1, v/v)=0.32. M.p.=168-170° C. ¹H NMR (400 MHz, DMSO-d₆) δ 8.72 (s, 1H, NH), 8.24 (d, J=8.8 Hz, 2H, CH_{Ar}), 7.50-7.39 (m, 4H, CH_{Ar}), 7.21-7.08 (m, 2H, CH_{Ar}), 6.71 (t, J=7.3 Hz, 1H, CH_{Ar}), 6.37 (s, 2H, NH₂), 5.19 (s, 2H, CH₂). ¹³C NMR (101 MHz, DMSO-d₆) δ 157.4, 154.4, 146.7, 145.4, 142.2, 128.4 (2C), 128.2 (2C), 123.6 (2C), 118.5, 115.5 (2C), 48.1. LCMS: t_{R}=10.18 min. [M+H]⁺=311.13. HPLC (λ₂₅₄): Purity 97.2%; t_{R}: 8.41 min (method 2).

### MCK289: 1-(2-nitrobenzyl)-N³-(pyridin-2-yl)-1H-1,2,4-triazole-3,5-diamine

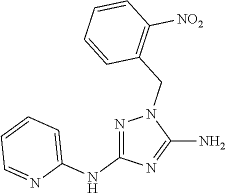 [SMILES: Nc1nc(Nc2ccccn2)nn1Cc1ccccc1[N+](=O)[O-]]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 2-Nitrobenzyl bromide (305 mg, 1.42 mmol), dissolved in degassed DMF (5 mL), was added dropwise and the reaction mixture was stirred at 0° C. for 1 h, then at r.t. for 4 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (94.1 mg, 0.303 mmol, 21% yield). Yellow solid, 94.1 mg, 0.303 mmol, 21% yield. Rf (CH₂Cl₂/MeOH:9/1, v/v)=0.22. M.p.=262° C. ¹H NMR (400 MHz, DMSO-d₆) δ 9.76 (s, 1H, NH), 8.20-8.10 (m, 2H, CH_{Ar}), 7.84 (d, J=8.5 Hz, 1H, CH_{Ar}), 7.74 (t, J=7.2 Hz, 1H, CH_{Ar}), 7.59 (ddd, J=14.3, 10.4, 4.5 Hz, 2H, CH_{Ar}), 6.98 (d, J=7.6 Hz, 1H, CH_{Ar}), 6.84-6.73 (m, 1H, CH_{Ar}), 6.56 (s, 2H, NH₂), 5.46 (s, 2H, CH₂). ¹³C NMR (101 MHz, DMSO-d₆) δ 156.2, 154.9, 154.1, 147.6, 147.3, 137.6, 134.2, 133.2, 128.5, 128.1, 124.9, 115.0, 109.6, 46.7. LCMS: t_{R}=2.68; 6.32 min. [M+H]⁺=312.13. HPLC (λ₂₅₄): Purity 98.6%; t_{R}: 5.23 min (method 1).

### MCK290: 1-(4-nitrobenzyl)-N³-(pyridin-2-yl)-1H-1,2,4-triazole-3,5-diamine

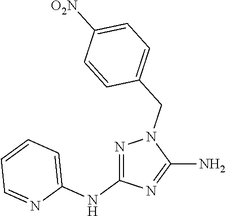 [SMILES: Nc1nc(Nc2ccccn2)nn1Cc1ccc([N+](=O)[O-])cc1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 4-Nitrobenzyl bromide (305 mg, 1.42 mmol), dissolved in degassed DMF (5 mL), was added dropwise and the reaction mixture was stirred at 0° C. for 1 h, then at r.t. for 4 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (107.3 mg, 0.345 mmol, 24% yield). Yellow solid, 107.3 mg, 0.345 mmol, 24% yield. Rf (CH₂Cl₂/MeOH:9/1, v/v)=0.28. M.p.=246° C. ¹H NMR (400 MHz, DMSO-d₆) δ 10.26 (s, 1H, NH), 8.28-8.22 (m, 2H, CH_{Ar}), 8.22-8.17 (m, 1H, CH_{Ar}), 7.85 (d, J=8.5 Hz, 1H, CH_{Ar}), 7.64 (ddd, J=8.8, 7.2, 2.0 Hz, 1H, CH_{Ar}), 7.48 (d, J=8.7 Hz, 2H, CH_{Ar}), 6.80 (ddd, J=7.2, 4.9, 1.0 Hz, 1H, CH_{Ar}), 6.72 (s, 2H, NH₂), 5.27 (s, 2H, CH₂). ¹³C NMR (101 MHz, DMSO-d₆) δ 156.0, 154.6, 154.0, 147.6, 146.7, 145.1, 137.9, 128.2 (2C), 123.7 (2C), 115.0, 109.6, 48.2. LCMS: t_{R}=2.69; 6.36 min. [M+H]⁺=312.13. HPLC (λ₂₅₄): Purity 98.8%; t_{R}: 5.43 min (method 1).

### MCK292: 1-(3-nitrobenzyl)-N³-(pyridin-2-yl)-1H-1,2,4-triazole-3,5-diamine

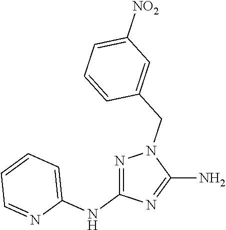 [SMILES: Nc1nc(Nc2ccccn2)nn1Cc1cccc([N+](=O)[O-])c1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 3-Nitrobenzyl chloride (305 mg, 1.42 mmol), dissolved in degassed DMF (5 mL), was added dropwise and the reaction mixture was stirred at r.t. for 3 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (89 mg, 0.286 mmol, 20% yield). Yellow solid, 89 mg, 0.286 mmol, 20% yield. Rf (CH₂Cl₂/MeOH:9/1, v/v)=0.32. M.p.=266° C. ¹H NMR (400 MHz, DMSO-d₆) δ 9.92 (s, 1H, NH), 8.21-8.12 (m, 3H, CH_{Ar}), 7.85 (dt, J=8.5, 1.0 Hz, 1H, CH_{Ar}), 7.73-7.67 (m, 2H, CH_{Ar}), 7.66-7.60 (m, 1H, CH_{Ar}), 6.79 (ddd, J=7.2, 4.9, 1.0 Hz, 1H, CH_{Ar}), 6.66 (s, 2H, NH₂), 5.24 (s, 2H, CH₂). ¹³C NMR (101 MHz, DMSO-d₆) δ 156.0, 154.5, 154.1, 147.8, 147.6, 139.6, 137.7, 134.0, 130.1, 122.3, 121.9, 115.0, 109.5, 48.0. LCMS: t_{R}=6.86 min. [M+H]⁺=312.07. HPLC (λ₂₅₄): Purity 99.5%; HPLC (λ₂₈₀): Purity 99.1%; t_{R}: 5.80 min (method 1).

### MCK295: 2-(5-amino-3-(pyridin-2-ylamino)-H-1,2,4-triazol-1-yl)-1-(3-nitrophenyl)ethan-1-one

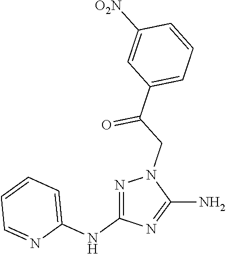 [SMILES: Nc1nc(Nc2ccccn2)nn1CC(=O)c1cccc([N+](=O)[O-])c1]

N³-(pyridin-2-yl)-1H-1,2,4-triazole-3,5-diamine (250 mg, 1.42 mmol) was dissolved in degassed DMF (10 mL), then potassium tert-butoxide (159 mg, 1.42 mmol) was added at 0° C. 2-Bromo-1-(3-nitrophenyl)ethan-1-one (344 mg, 1.42 mmol), dissolved in degassed DMF (5 mL), was added dropwise and the reaction mixture was stirred at 0° C. for 1 h, then at r.t. for 4 h. Water was added to the mixture reaction and was extracted with ethyl acetate, washed with water, dried over magnesium sulfate and the solvents were evaporated under reduced pressure. Purification by silica gel flash chromatography (CH₂Cl₂/MeOH, 10/0 then 9/1) afforded the titled compound as a yellow solid (56.4 mg, 0.166 mmol, 12% yield). Yellow solid, 56.4 mg, 0.166 mmol, 12% yield. Rf (CH₂Cl₂/MeOH:9/1, v/v)=0.30. M.p.=240° C. ¹H NMR (400 MHz, DMSO-d₆) δ 10.22 (s, 1H, NH), 8.74 (t, J=1.9 Hz, 1H, CH_{Ar}), 8.57-8.50 (m, 1H, CH_{Ar}), 8.47 (d, J=7.8 Hz, 1H, CH_{Ar}), 8.19-8.13 (m, 1H, CH_{Ar}), 7.94-7.83 (m, 2H, CH_{Ar}), 7.62 (ddd, J=8.8, 7.1, 2.0 Hz, 1H, CH_{Ar}), 6.79 (ddd, J=7.2, 4.9, 1.0 Hz, 1H, CH_{Ar}), 6.55 (s, 2H, NH₂), 5.66 (s, 2H, CH₂). ¹³C NMR (101 MHz, DMSO-d₆) δ 192.0, 155.7, 155.5, 154.1, 148.0, 147.5, 137.8, 135.8, 134.2, 130.7, 127.9, 122.4, 114.9, 109.6, 53.1. LCMS: t_{R}=2.76; 6.66 min. [M+H]⁺=340.07. HPLC (λ₂₅₄): Purity 96.1%; t_{R}: 5.71 min (method 1).

### MCK297: 1-(2-nitrobenzyl)-N³-phenyl-1H-1,2,4-triazole-3,5-diamine

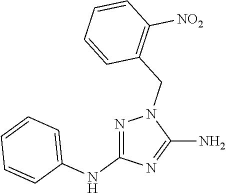 [SMILES: Nc1nc(Nc2ccccc2)nn1Cc1ccccc1[N+](=O)[O-]]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 2-Nitrobenzyl bromide (307 mg, 1.43 mmol), dissolved in degassed DMF (5 mL), was added dropwise and the reaction mixture was stirred at 0° C. for 30 min, then at r.t. for 3 h. Water was added to the mixture reaction and was extracted with ethyl acetate, washed with water, dried over magnesium sulfate and the solvents were evaporated under reduced pressure. Purification by reversed-phase chromatography (H₂O/CH₃CN, 10/0 then 60/40) afforded the titled compound as a yellow solid (60.9 mg, 0.196 mmol, 14% yield). Yellow solid, 60.9 mg, 0.196 mmol, 14% yield. Rf (CH₂Cl₂/THF, 1/1, v/v)=0.57. M.p.=78-80° C. ¹H NMR (400 MHz, DMSO-d₆) δ 8.76 (s, 1H, NH), 8.14 (dd, J=8.2, 1.3 Hz, 11H, CH_{Ar}), 7.73 (td, J=7.6, 1.3 Hz, 1H, CH_{Ar}), 7.62-7.52 (m, 1H, CH_{Ar}), 7.47 (dd, J=8.7, 1.2 Hz, 2H, CH_{Ar}), 7.14 (dd, J=8.6, 7.2 Hz, 2H, CH_{Ar}), 6.99 (dd, J=7.9, 1.3 Hz, 1H, CH_{Ar}), 6.72 (tt, J=7.3, 1.2 Hz, 1H, CH_{Ar}), 6.34 (s, 2H, NH₂), 5.41 (s, 2H, CH₂). ¹³C NMR (101 MHz, DMSO-d₆) δ 157.5, 154.6, 147.3, 142.2, 134.2, 133.5, 128.5, 128.4 (2C), 128.2, 124.8, 118.5, 115.6 (2C), 46.6. LCMS: t_{R}=10.47 min. [M+H]⁺=311.07. HPLC (λ₂₈₂): Purity 97.2%; t_{R}: 8.21 min (method 2).

### MCK298: N³-phenyl-1-(3,4,5-trimethoxybenzyl)-1H-1,2,4-triazole-3,5-diamine

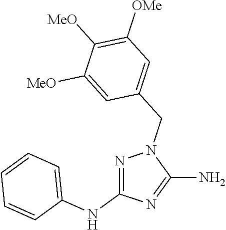 [SMILES: COc1cc(Cn2nc(Nc3ccccc3)nc2N)cc(OC)c1OC]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 5-(Chloromethyl)-1,2,3-trimethoxybenzene (309 mg, 1.43 mmol), dissolved in degassed DMF (5 mL), was added dropwise and the reaction mixture was stirred at 0° C. for 30 min, then at r.t. for 3 h. Water was added to the mixture reaction and was extracted with ethyl acetate, washed with water, dried over magnesium sulfate and the solvents were evaporated under reduced pressure. Purification by reversed-phase chromatography (H₂O/CH₃CN, 10/0 then 60/40) afforded the titled compound as a white solid (103.5 mg, 0.292 mmol, 20% yield). White solid, 103.5 mg, 0.292 mmol, 20% yield. Rf (CH₂Cl₂/THF, 1/1, v/v)=0.48. M.p.=168-170° C. ¹H NMR (400 MHz, DMSO-d₆) δ 8.65 (s, 1H, NH), 7.49 (dd, J=8.6, 1.2 Hz, 2H, CH_{Ar}), 7.21-7.07 (m, 2H, CH_{Ar}), 6.71 (tt, J=7.3, 1.2 Hz, 1H, CH_{Ar}), 6.66 (s, 2H, CH_{Ar}), 6.27 (s, 2H, NH₂), 4.92 (s, 2H, CH₂), 3.74 (s, 6H, CH₃), 3.63 (s, 3H, CH₃). ¹³C NMR (101 MHz, DMSO-d₆) δ 157.0, 153.9, 152.7 (2C), 142.4, 136.7, 133.0, 128.4 (2C), 118.4, 115.4 (2C), 105.0 (2C), 59.9, 55.7 (2C), 48.8. LCMS: t_{R}=9.36 min. [M+H]⁺=355.95. HPLC (λ₂₅₄): Purity 99.7%; HPLC (λ₂₈₂): Purity 98.8%; t_{R}: 7.44 min (method 2).

### MCK299: 2-(5-amino-3-(phenylamino)-1H-1,2,4-triazol-1-yl)-1-phenylethan-1-one

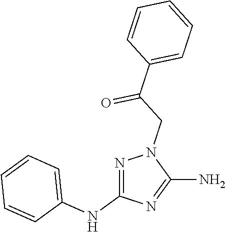 [SMILES: Nc1nc(Nc2ccccc2)nn1CC(=O)c1ccccc1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. 2-Bromo-1-phenylethan-1-one (283 mg, 1.43 mmol), dissolved in degassed DMF (5 mL), was added dropwise and the reaction mixture was stirred at 0° C. for 30 min, then at r.t. for 2 h. Water was added to the mixture reaction and was extracted with ethyl acetate, washed with water, dried over magnesium sulfate and the solvents were evaporated under reduced pressure. Purification by reversed-phase chromatography (H₂O/CH₃CN, 10/0 then 60/40) afforded the titled compound as a white solid (139.2 mg, 0.475 mmol, 33% yield). White solid, 139.2 mg, 0.475 mmol, 33% yield. Rf (CH₂Cl₂/THF, 1/1, v/v)=0.50. M.p.=170-172° C. ¹H NMR (400 MHz, DMSO-d₆) δ 8.65 (s, 1H, NH), 8.09-7.99 (m, 2H, CH_{Ar}), 7.70 (t, J=7.4 Hz, 1H, CH_{Ar}), 7.59 (t, J=7.7 Hz, 2H, CH_{Ar}), 7.46 (d, J=7.7 Hz, 2H, CH_{Ar}), 7.13 (t, J=7.9 Hz, 2H, CH_{Ar}), 6.70 (t, J=7.3 Hz, 1H, CH_{Ar}), 6.16 (s, 2H, NH₂), 5.45 (s, 2H, CH₂). ¹³C NMR (101 MHz, DMSO-d₆) δ 193.2, 157.0, 155.2, 142.3, 134.6, 133.7, 128.8 (2C), 128.3 (2C), 128.0 (2C), 118.3, 115.5 (2C), 52.6. LCMS: t_{R}=9.80 min. [M+H]⁺=294.07. HPLC (λ₂₅₄): Purity 97.6%; HPLC (λ₂₈₀): Purity 94.2%; t_{R}: 7.73 min (method 2).

### MCK300: 1-phenethyl-N⁵-phenyl-1H-1,2,4-triazole-3,5-diamine

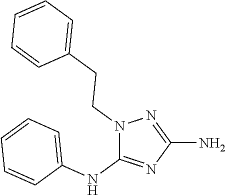 [SMILES: Nc1nc(Nc2ccccc2)n(CCc2ccccc2)n1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. (2-Chloroethyl)benzene (0.19 mL, 1.43 mmol), dissolved in degassed DMF (5 mL), was added dropwise and the reaction mixture was stirred at 0° C., then potassium iodide (1.19 g, 7.14 mmol) was added at r.t. for 24 h. Water was added to the mixture reaction and was extracted with ethyl acetate, washed with water, dried over magnesium sulfate and the solvents were evaporated under reduced pressure. Purification by reversed-phase chromatography (H₂O/CH₃CN, 10/0 then 60/40) afforded the titled compound as a white solid (12.7 mg, 0.046 mmol, 3% yield) along with the N₃-benzylated regioisomer. White solid, 12.7 mg, 0.046 mmol, 3% yield. Rf (CH₂Cl₂/THF, 1/1, v/v)=0.57. M.p.=158-160° C. ¹H NMR (400 MHz, DMSO-d₆) δ 8.48 (s, 1H, NH), 7.50-7.44 (m, 2H, CH_{Ar}), 7.31-7.14 (m, 7H, CH_{Ar}), 6.88-6.81 (m, 1H, CH_{Ar}), 5.11 (s, 2H, NH₂), 4.12-4.04 (m, 2H, CH₂), 3.03-2.94 (m, 2H, CH₂). ¹³C NMR (101 MHz, DMSO-d₆) δ 160.4, 149.6, 141.1, 138.4, 128.7 (2C), 128.4 (2C), 128.2 (2C), 126.2, 120.2, 116.9 (2C), 46.4, 34.8. LCMS: t_{R}=9.51 min. [M+H]⁺=280.07. HPLC (λ₂₅₄): Purity 94.2%; t_{R}: 6.47 min (method 2).

### MCK301: 1-phenethyl-N³-phenyl-1H-1,2,4-triazole-3,5-diamine

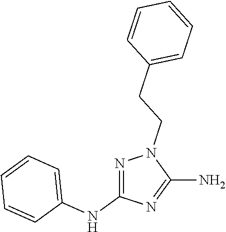 [SMILES: Nc1nc(Nc2ccccc2)nn1CCc1ccccc1]

N³-phenyl-1H-1,2,4-triazole-3,5-diamine (250 mg, 1.43 mmol) was dissolved in degassed DMF (10 mL), then potassium tert-butoxide (159 mg, 1.43 mmol) was added. (2-chloroethyl)benzene (0.19 mL, 1.43 mmol), dissolved in degassed DMF (5 mL), was added dropwise and the reaction mixture was stirred at 0° C., then potassium iodide (1.19 g, 7.14 mmol) was added at r.t. for 24 h. Water was added to the mixture reaction and was extracted with ethyl acetate, washed with water, dried over magnesium sulfate and the solvents were evaporated under reduced pressure. Purification by reversed-phase chromatography (H₂O/CH₃CN, 10/0 then 60/40) afforded the titled compound as a white solid (37.6 mg, 0.135 mmol, 9% yield) along with the N₅-benzylated regioisomer. White solid, 37.6 mg, 0.135 mmol, 9% yield. Rf (CH₂Cl₂/THF, 1/1, v/v)=0.56. M.p.=160-162° C. ¹H NMR (400 MHz, DMSO-d₆) δ 8.61 (s, 1H, NH), 7.53-7.43 (m, 2H, CH_{Ar}), 7.28 (h, J=5.7 Hz, 4H, CH_{Ar}), 7.21 (td, J=6.0, 2.5 Hz, 1H, CH_{Ar}), 7.16 (ddd, J=8.5, 5.7, 2.1 Hz, 2H, CH_{Ar}), 6.77-6.65 (m, 1H, CH_{Ar}), 6.06 (s, 2H, NH₂), 4.06-3.91 (m, 2H, CH₂), 3.04-2.92 (m, 2H, CH₂). ¹³C NMR (101 MHz, DMSO-d₆) δ 156.7, 153.5, 142.4, 138.5, 128.8 (2C), 128.4 (2C), 128.2 (2C), 126.2, 118.3, 115.5 (2C), 46.4, 34.6. LCMS: t_{R}=9.58 min. [M+H]⁺=280.07. HPLC (λ₂₅₄): Purity 97.3%; t_{R}: 7.00 min (method 2).

### MCK338: N⁵-phenyl-1-(3-(trifluoromethyl)benzyl)-1H-1,2,4-triazole-3,5-diamine

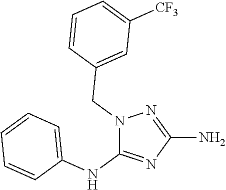 [SMILES: Nc1nc(Nc2ccccc2)n(Cc2cccc(C(F)(F)F)c2)n1]

N³-phenyl-1H-1,2,4-triazole-3,5-diamine (250 mg, 1.43 mmol) was dissolved in anhydrous DMF (1 mL), then potassium tert-butoxide (160 mg, 1.43 mmol) was added at 0° C. 3-(Trifluoromethyl)benzyl chloride (0.23 mL, 1.50 mmol), dissolved in anhydrous CH₂Cl₂ (1 mL), was added dropwise and the reaction mixture was stirred at 0° C. for 1 h and at r.t. for 2 h. The reaction mixture was quenched with water (1 mL) and the solvents were evaporated under reduced pressure. Purification by reversed-phase chromatography (H₂O/CH₃CN, 10/0 then 45/55) afforded the titled compound as a white solid (51.3 mg, 0.154 mmol, 11% yield) along with the N₃-benzylated regioisomer. White solid, 51.3 mg, 0.154 mmol, 11% yield. Rf (CH₂Cl₂/THF, 1/1, v/v)=0.83. M.p.=128-130° C. ¹H NMR (400 MHz, DMSO-d₆) δ 8.85 (s, 1H, NH), 7.68-7.60 (m, 2H, CH_{Ar}), 7.60-7.55 (m, 3H, CH_{Ar}), 7.49 (d, J=7.6 Hz, 1H, CH_{Ar}), 7.24 (t, J=7.9 Hz, 2H, CH_{Ar}), 6.88 (t, J=7.3 Hz, 1H, CH_{Ar}), 5.23 (s, 2H, NH₂), 5.17 (d, J=9.4 Hz, 2H, CH₂). ¹³C NMR (101 MHz, DMSO-d₆) δ 160.9, 150.4, 140.9, 139.0, 131.3, 129.6, 129.2, 128.9, 128.6 (2C), 124.0, 123.7, 120.6, 117.0 (2C), 48.2. LCMS: t_{R}=10.83 min. [M+H]⁺=334.16. HPLC (λ₂₈₀): Purity 94.5%; t_{R}: 10.7 min (method 2).

### MCK339: 1-(3-bromobenzyl)-N⁵-phenyl-1H-1,2,4-triazole-3,5-diamine

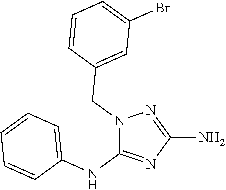 [SMILES: Nc1nc(Nc2ccccc2)n(Cc2cccc(Br)c2)n1]

N³-phenyl-1H-1,2,4-triazole-3,5-diamine (150 mg, 0.857 mmol) was dissolved in anhydrous DMF (1 mL), then potassium tert-butoxide (96 mg, 0.857 mmol) was added at 0° C. 3-Bromobenzyl bromide (225 mg, 0.857 mmol), dissolved in anhydrous CH₂Cl₂ (1 mL), was added dropwise and the reaction mixture was stirred at 0° C. for 1 h and at r.t. for an additional 1 h. The reaction mixture was quenched with water (1 mL) and the solvents were evaporated under reduced pressure. Purification by reversed-phase chromatography (H₂O/CH₃CN, 10/0 then 45/55) afforded the titled compound as a white solid (29.5 mg, 0.086 mmol, 10% yield) along with the N₃-benzylated regioisomer. White solid, 29.5 mg, 0.086 mmol, 10% yield. Rf (CH₂Cl₂/THF, 1/1, v/v)=0.63. M.p.=146-148° C. ¹H NMR (400 MHz, DMSO-d₆) δ 8.81 (s, 1H, NH), 7.62-7.55 (m, 2H, CH_{Ar}), 7.50-7.45 (m, 1H, CH_{Ar}), 7.39 (t, J=1.8 Hz, 1H, CH_{Ar}), 7.31 (t, J=7.8 Hz, 1H, CH_{Ar}), 7.27-7.18 (m, 3H, CH_{Ar}), 6.88 (tt, J=7.4, 1.1 Hz, 1H, CH_{Ar}), 5.16 (s, 2H, NH₂), 5.13 (s, 2H, CH₂). ¹³C NMR (101 MHz, DMSO-d₆) δ 160.8, 150.3, 140.9, 140.3, 130.6, 130.0, 129.9, 128.6 (2C), 126.2, 121.6, 120.5, 116.9 (2C), 48.0. LCMS: t_{R}=8.47 min. [M+H]⁺=342.30; 343.90. HPLC (λ₂₈₀): Purity 96.5%; t_{R}: 10.5 min (method 3).

### MCK340: 1-(3-bromobenzyl)-N³-(4-methoxyphenyl)-1H-1,2,4-triazole-3,5-diamine

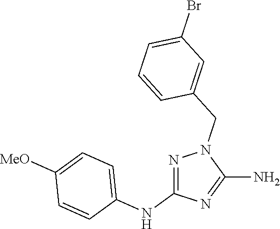 [SMILES: COc1ccc(Nc2nc(N)n(Cc3cccc(Br)c3)n2)cc1]

N³-(4-methoxyphenyl)-1H-1,2,4-triazole-3,5-diamine (250 mg, 1.22 mmol) was dissolved in anhydrous DMF (1 mL), then potassium tert-butoxide (137 mg, 1.22 mmol) was added at 0° C. 3-Bromobenzyl bromide (320 mg, 1.28 mmol), dissolved in anhydrous CH₂Cl₂ (1 mL), was added dropwise and the reaction mixture was stirred at 0° C. for 1 h and at r.t. for 2 h. The reaction mixture was quenched with water (1 mL) and the solvents were evaporated under reduced pressure. Purification by reversed-phase chromatography (H₂O/CH₃CN, 10/0 then 45/55) afforded the titled compound as a beige solid (88.4 mg, 0.237 mmol, 19% yield). Beige solid, 88.4 mg, 0.237 mmol, 19% yield. Rf (CH₂Cl₂/THF, 1/1, v/v)=0.35. M.p.=96-98° C. ¹H NMR (400 MHz, DMSO-d₆) δ 8.43 (s, 1H, NH), 7.47 (ddd, J=7.9, 2.0, 1.1 Hz, 1H, CH_{Ar}), 7.42 (t, J=1.8 Hz, 1H, CH_{Ar}), 7.41-7.35 (m, 2H, CH_{Ar}), 7.32 (t, J=7.8 Hz, 1H, CH_{Ar}), 7.24 (dt, J=7.8, 1.4 Hz, 1H, CH_{Ar}), 6.82-6.70 (m, 2H, NH₂), 6.28 (s, 2H, CH₂), 5.01 (s, 2H), 3.66 (s, 3H, OCH₃). ¹³C NMR (101 MHz, DMSO-d₆) δ 157.6, 154.1, 152.1, 140.4, 136.0, 130.6, 130.0, 129.9, 126.3, 121.5, 116.7 (2C), 113.8 (2C), 55.1, 47.9. LCMS: t_{R}=8.63 min. [M+H]⁺=374.30; 376.40. HPLC (λ₂₅₄): Purity 100%; HPLC (λ₂₈₀): Purity 98.8%; t_{R}: 10.5 min (method 3).

### MCK341: 1-(3-bromobenzyl)-NA-(4-methylphenyl)-1H-1,2,4-triazole-3,5-diamine

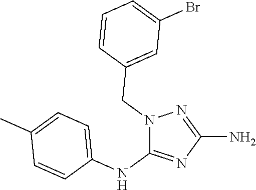 [SMILES: Cc1ccc(Nc2nc(N)nn2Cc2cccc(Br)c2)cc1]

N³-(4-methylphenyl)-1H-1,2,4-triazole-3,5-diamine (250 mg, 1.32 mmol) was dissolved in anhydrous DMF (1 mL), then potassium tert-butoxide (148 mg, 1.32 mmol) was added at 0° C. 3-Bromobenzyl bromide (347 mg, 1.39 mmol), dissolved in anhydrous CH₂Cl₂ (1 mL), was added dropwise and the reaction mixture was stirred at 0° C. for 1 h and at r.t. for 2 h. The reaction mixture was quenched with water (1 mL) and the solvents were evaporated under reduced pressure. Purification by reversed-phase chromatography (H₂O/CH₃CN, 10/0 then 45/55) afforded the titled compound as a pale pink solid (47.7 mg, 0.134 mmol, 10% yield) along with the N₃-benzylated regioisomer. Pale pink solid, 47.7 mg, 0.134 mmol, 10% yield. Rf (CH₂Cl₂/THF, 1/1, v/v)=0.54. M.p.=162-164° C. ¹H NMR (400 MHz, DMSO-d₆) δ 8.70 (s, 1H, NH), 7.47 (dd, J=8.8, 2.1 Hz, 3H, CH_{Ar}), 7.38 (t, J=1.8 Hz, 1H, CH_{Ar}), 7.31 (t, J=7.8 Hz, 1H, CH_{Ar}), 7.22-7.18 (m, 1H, CH_{Ar}), 7.08-7.02 (m, 2H, CH_{Ar}), 5.14 (s, 2H, NH₂), 5.11 (s, 2H, CH₂), 2.22 (s, 3H, CH₃). ¹³C NMR (101 MHz, DMSO-d₆)) S 160.8, 150.5, 140.3, 138.4, 130.6, 130.0, 129.9, 129.2, 129.0 (2C), 126.2, 121.6, 117.1 (2C), 47.9, 20.2. LCMS: t_{R}=9.06 min. [M+H]⁺=357.9; 358.9. HPLC (λ₂₅₄): Purity 99.3%; HPLC (λ₂₈₀): Purity 99.7%; t_{R}: 11.0 min (method 3).

### MCK342: 1-(3-bromobenzyl)-N³-(4-methylphenyl)-1H-1,2,4-triazole-3,5-diamine

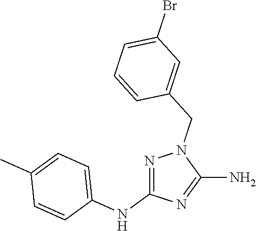 [SMILES: Cc1ccc(Nc2nc(N)n(Cc3cccc(Br)c3)n2)cc1]

N³-(4-methylphenyl)-1H-1,2,4-triazole-3,5-diamine (250 mg, 1.32 mmol) was dissolved in anhydrous DMF (1 mL), then potassium tert-butoxide (148 mg, 1.32 mmol) was added at 0° C. 3-Bromobenzyl bromide (347 mg, 1.39 mmol), dissolved in anhydrous CH₂Cl₂ (1 mL), was added dropwise and the reaction mixture was stirred at 0° C. for 1 h and at r.t. for 2 h. The reaction mixture was quenched with water (1 mL) and the solvents were evaporated under reduced pressure. Purification by reversed-phase chromatography (H₂O/CH₃CN, 10/0 then 45/55) afforded the titled compound as a yellow solid (157.4 mg, 0.441 mmol, 33% yield) along with the N₅-benzylated regioisomer. Yellow solid, 157.4 mg, 0.441 mmol, 33% yield. Rf (CH₂Cl₂/THF, 1/1, v/v)=0.46. M.p.=86-88° C. ¹H NMR (400 MHz, DMSO-d₆) δ 8.54 (s, 1H, NH), 7.50-7.45 (m, 1H, CH_{Ar}), 7.43 (t, J=1.8 Hz, 1H, CH_{Ar}), 7.37-7.29 (m, 3H, CH_{Ar}), 7.26-7.21 (m, 1H, CH_{Ar}), 7.02-6.90 (m, 2H, CH_{Ar}), 6.30 (s, 2H, NH₂), 5.02 (s, 2H, CH₂), 2.18 (s, 3H, CH₃). ¹³C NMR (101 MHz, DMSO-d₆) δ 157.4, 154.1, 140.4, 139.8, 130.6, 130.0, 129.9, 128.8 (2C), 126.8, 126.3, 121.5, 115.5 (2C), 47.9, 20.2. LCMS: t_{R}=9.57 min. [M+H]⁺=357.40; 358.40. HPLC (λ₂₅₄): Purity 96.6%; HPLC (λ₂₈₀): Purity 95.1%; t_{R}: 11.5 min (method 3).

### MCK343: 1-(3-bromobenzyl)-N⁵-(4-chlorophenyl)-1H-1,2,4-triazole-3,5-diamine

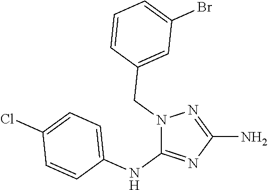 [SMILES: Nc1nc(Nc2ccc(Cl)cc2)n(Cc2cccc(Br)c2)n1]

N³-(4-chlorophenyl)-1H-1,2,4-triazole-3,5-diamine (250 mg, 1.20 mmol) was dissolved in anhydrous DMF (1 mL), then potassium tert-butoxide (134 mg, 1.20 mmol) was added at 0° C. 3-Bromobenzyl bromide (310 mg, 1.26 mmol), dissolved in anhydrous CH₂Cl₂ (1 mL), was added dropwise and the reaction mixture was stirred at 0° C. for 1 h and at r.t. for 2 h. The reaction mixture was quenched with water (1 mL) and the solvents were evaporated under reduced pressure. Purification by reversed-phase chromatography (H₂O/CH₃CN, 10/0 then 45/55) afforded the titled compound as a pale pink solid (22.4 mg, 0.059 mmol, 5% yield) along with the N₃-benzylated regioisomer. Pale pink solid, 22.4 mg, 0.059 mmol, 5% yield. Rf (CH₂Cl₂/THF, 1/1, v/v)=0.54. M.p.=82-84° C. ¹H NMR (400 MHz, DMSO-d₆) δ 9.00 (s, 1H, NH), 7.68-7.57 (m, 2H, CH_{Ar}), 7.47 (ddd, J=8.0, 2.1, 1.1 Hz, 1H, CH_{Ar}), 7.38 (t, J=1.8 Hz, 1H, CH_{Ar}), 7.36-7.27 (m, 3H, CH_{Ar}), 7.19 (dt, J=7.8, 1.3 Hz, 1H, CH_{Ar}), 5.20 (s, 2H, NH₂), 5.12 (s, 2H, CH₂). ¹³C NMR (101 MHz, DMSO-d₆) δ 160.8, 149.8, 140.1, 139.9, 130.6, 130.1, 129.9, 128.4 (2C), 126.2, 124.0, 121.6, 118.4 (2C), 48.0. LCMS: t_{R}=9.76 min. [M+H]⁺=378.4; 380.3. HPLC (λ₂₅₄): Purity 96.4%; HPLC (λ₂₈₀): Purity 98.7%; t_{R}: 11.9 min (method 3).

### MCK344: 1-(3-bromobenzyl)-N³-(4-chlorophenyl)-1H-1,2,4-triazole-3,5-diamine

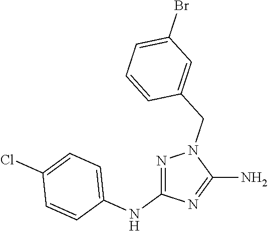 [SMILES: Nc1nc(Nc2ccc(Cl)cc2)nn1Cc1cccc(Br)c1]

N³-(4-chlorophenyl)-1H-1,2,4-triazole-3,5-diamine (250 mg, 1.20 mmol) was dissolved in anhydrous DMF (1 mL), then potassium tert-butoxide (134 mg, 1.20 mmol) was added. 3-Bromobenzyl bromide (310 mg, 1.26 mmol), dissolved in anhydrous CH₂Cl₂ (1 mL), was added dropwise and the reaction mixture was stirred at 0° C. for 1 h and at r.t. for 2 h. The reaction mixture was quenched with water (1 mL) and the solvents were evaporated under reduced pressure. Purification by reversed-phase chromatography (H₂O/CH₃CN, 10/0 then 45/55) afforded the titled compound as an orange solid (197.7 mg, 0.524 mmol, 44% yield) along with the N₅-benzylated regioisomer. Orange solid, 197.7 mg, 0.524 mmol, 44% yield. Rf (CH₂Cl₂/THF, 1/1, v/v)=0.42. M.p.=124-126° C. ¹H NMR (400 MHz, DMSO-d₆) δ 8.93 (s, 1H NH), 7.53-7.45 (m, 3H, CH_{Ar}), 7.43 (t, J=1.8 Hz, 1H, CH_{Ar}), 7.32 (t, J=7.8 Hz, 1H, CH_{Ar}), 7.27-7.22 (m, 1H, CH_{Ar}), 7.22-7.16 (m, 2H, CH_{Ar}), 6.38 (s, 2H, NH₂), 5.04 (s, 2H, CH₂). ¹³C NMR (101 MHz, DMSO-d₆) δ 157.0, 154.3, 141.2, 140.2, 130.6, 130.1, 129.9, 128.2 (2C), 126.3, 121.8, 121.6, 116.9 (2C), 48.0. LCMS: t_{R}=10.52 min. [M+H]⁺=378.4; 380.3. HPLC (λ₂₅₄): Purity 98.5%; HPLC (λ₂₈₀): Purity 98.7%; t_{R}: 12.6 min (method 3).

### MCK345: 1-(3-bromobenzyl)-N⁵-(4-bromophenyl)-1H-1,2,4-triazole-3,5-diamine

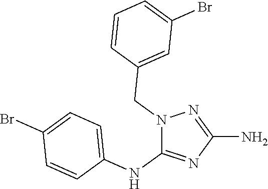 [SMILES: Nc1nc(Nc2ccc(Br)cc2)n(Cc2cccc(Br)c2)n1]

N³-(4-bromophenyl)-1H-1,2,4-triazole-3,5-diamine (250 mg, 0.988 mmol) was dissolved in anhydrous DMF (1 mL), then potassium tert-butoxide (111 mg, 0.988 mmol) was added at 0° C. 3-Bromobenzyl bromide (259 mg, 1.04 mmol), dissolved in anhydrous CH₂Cl₂ (1 mL), was added dropwise and the reaction mixture was stirred at 0° C. for 1 h and at r.t. for 2 h. The reaction mixture was quenched with water (1 mL) and the solvents were evaporated under reduced pressure. Purification by reversed-phase chromatography (H₂O/CH₃CN, 10/0 then 45/55) afforded the titled compound as a pink solid (3.5 mg, 0.008 mmol, 1% yield) along with the N₃-benzylated regioisomer. Pink solid (3.5 mg, 0.008 mmol, 1% yield. Rf (CH₂Cl₂/THF, 1/1, v/v)=0.54. M.p.=76-78° C. ¹H NMR (400 MHz, DMSO-d₆) δ 9.01 (s, 1H, NH), 7.59-7.55 (m, 2H, CH_{Ar}), 7.47 (ddd, J=8.0, 2.2, 1.1 Hz, 1H, CH_{Ar}), 7.44-7.37 (m, 3H, CH_{Ar}), 7.31 (t, J=7.8 Hz, 1H, CH_{Ar}), 7.19 (dt, J=7.8, 1.3 Hz, 1H, CH_{Ar}), 5.20 (d, J=7.7 Hz, 2H, NH₂), 5.12 (s, 2H, CH₂). ¹³C NMR (101 MHz, DMSO-d₆) δ 160.8, 149.8, 140.3, 140.1, 131.3 (2C), 130.6, 130.1, 129.9, 126.2, 121.6, 118.8 (2C), 111.8, 48.1. LCMS: t_{R}=10.01 min. [M+H]⁺=422.3; 424.4. HPLC (λ₂₈₀): Purity 95.1%; t_{R}: 12.2 min (method 3).

### MCK346: 1-(3-bromobenzyl)-N³-(4-bromophenyl)-1H-1,2,4-triazole-3,5-diamine

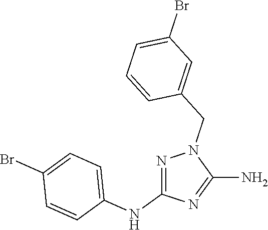 [SMILES: Nc1nc(Nc2ccc(Br)cc2)nn1Cc1cccc(Br)c1]

N³-(4-bromophenyl)-1H-1,2,4-triazole-3,5-diamine (250 mg, 0.988 mmol) was dissolved in anhydrous DMF (1 mL), then potassium tert-butoxide (111 mg, 0.988 mmol) was added at 0° C. 3-Bromobenzyl bromide (259 mg, 1.04 mmol), dissolved in anhydrous CH₂Cl₂ (1 mL), was added dropwise and the reaction mixture was stirred at 0° C. for 1 h and at r.t. for 2 h. The reaction mixture was quenched with water (1 mL) and the solvents were evaporated under reduced pressure. Purification by reversed-phase chromatography (H₂O/CH₃CN, 10/0 then 45/55) afforded the titled compound as a pink solid (132.4 mg, 0.314 mmol, 32% yield) along with the N₅-benzylated regioisomer. Pink solid (132.4 mg, 0.314 mmol, 32% yield. Rf (CH₂Cl₂/THF, 1/1, v/v)=0.42. M.p.=130-132° C. ¹H NMR (400 MHz, DMSO-d₆) δ 8.94 (s, 1H, NH), 7.50-7.45 (m, 1H, CH_{Ar}), 7.45-7.41 (m, 3H, CH_{Ar}), 7.35-7.28 (m, 3H, CH_{Ar}), 7.26-7.21 (m, 1H, CH_{Ar}), 6.38 (s, 2H, NH₂), 5.04 (s, 2H, CH₂). ¹³C NMR (101 MHz, DMSO-d₆) δ 156.9, 154.3, 141.6, 140.2, 131.0 (2C), 130.6, 130.1, 129.9, 126.3, 121.6, 117.5 (2C), 109.5, 48.0. LCMS: t_{R}=10.64 min. [M+H]⁺=422.3; 424.2. HPLC (λ₂₅₄): Purity 97.7%; HPLC (λ₂₈₀): Purity 98.8%; t_{R}: 13.0 min (method 3).

### MCK347: methyl-4-((3-amino-1-(3-bromobenzyl)-1H-1,2,4-triazol-5-yl)amino)benzoate

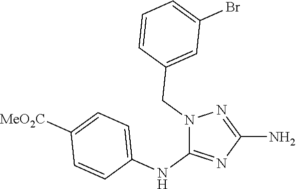 [SMILES: COC(=O)c1ccc(Nc2nc(N)nn2Cc2cccc(Br)c2)cc1]

Methyl-4-((5-amino-1H-1,2,4-triazol-3-yl)amino)benzoate (250 mg, 1.07 mmol) was dissolved in anhydrous DMF (1 mL), then potassium tert-butoxide (120 mg, 1.07 mmol) was added at 0° C. 3-Bromobenzyl bromide (280 mg, 1.13 mmol), dissolved in anhydrous CH₂Cl₂ (1 mL), was added dropwise and the reaction mixture was stirred at 0° C. for 1 h and at r.t. for 5 h 30. The reaction mixture was quenched with water (1 mL) and the solvents were evaporated under reduced pressure. Purification by reversed-phase chromatography (H₂O/CH₃CN, 10/0 then 45/55) afforded the titled compound as an off-white solid (9.9 mg, 0.025 mmol, 2% yield) along with the N₃-benzylated regioisomer. Rf (CH₂Cl₂/THF, 1/1, v/v)=0.56. M.p.=100° C. ¹H NMR (400 MHz, DMSO-d₆) δ 9.33 (s, 1H, NH), 7.90-7.82 (m, 2H, CH_{Ar}), 7.72-7.64 (m, 2H, CH_{Ar}), 7.47 (ddd, J=8.0, 2.1, 1.1 Hz, 1H, CH_{Ar}), 7.39 (t, J=1.8 Hz, 1H, CH_{Ar}), 7.31 (t, J=7.8 Hz, 1H, CH_{Ar}), 7.22-7.15 (m, 1H, CH_{Ar}), 5.28 (s, 2H, NH₂), 5.16 (s, 2H, CH₂), 3.80 (s, 3H, CH₃). ¹³C NMR (101 MHz, DMSO-d₆) δ 166.0, 161.0, 145.4, 140.0, 130.6 (2C), 130.3 (2C), 130.1, 129.9 (2C), 126.2, 121.6, 115.9 (2C), 51.6, 48.2. LCMS: t_{R}=8.92 min. [M+H]⁺=400.4; 401.8. HPLC (λ₂₈₀): Purity 96.7%; t_{R}: 11.0 min (method 3).

### MCK348: methyl-4-((5-amino-1-(3-bromobenzyl)-1H-1,2,4-triazol-3-yl)amino)benzoate

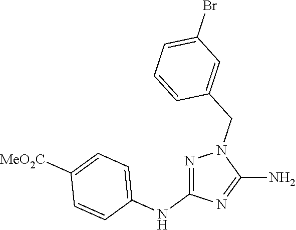 [SMILES: COC(=O)c1ccc(Nc2nc(N)n(Cc3cccc(Br)c3)n2)cc1]

Methyl-4-((5-amino-1H-1,2,4-triazol-3-yl)amino)benzoate (250 mg, 1.07 mmol) was dissolved in anhydrous DMF (1 mL), then potassium tert-butoxide (120 mg, 1.07 mmol) was added at 0° C. 3-Bromobenzyl bromide (280 mg, 1.13 mmol), dissolved in anhydrous CH₂Cl₂ (1 mL), was added dropwise and the reaction mixture was stirred at 0° C. for 1 h and at r.t. for 5 h 30. The reaction mixture was quenched with water (1 mL) and the solvents were evaporated under reduced pressure. Purification by reversed-phase chromatography (H₂O/CH₃CN, 10/0 then 45/55) afforded the titled compound as an off-white solid (83.7 mg, 0.209 mmol, 20% yield) along with the N₅-benzylated regioisomer. Rf (CH₂Cl₂/THF, 1/1, v/v)=0.39. M.p.=182° C. ¹H NMR (400 MHz, DMSO-d₆) δ 9.37 (s, 1H, NH), 7.81-7.75 (m, 2H, CH_{Ar}), 7.56-7.51 (m, 2H, CH_{Ar}), 7.48 (ddd, J=7.9, 2.1, 1.1 Hz, 1H, CH_{Ar}), 7.44 (t, J=1.8 Hz, 1H, CH_{Ar}), 7.33 (t, J=7.8 Hz, 1H, CH_{Ar}), 7.25 (dt, J=7.8, 1.3 Hz, 1H, CH_{Ar}), 6.45 (s, 2H, NH₂), 5.06 (s, 2H, CH₂), 3.77 (s, 3H, CH₃). ¹³C NMR (101 MHz, DMSO-d₆) δ 166.1, 156.5, 154.3, 146.6, 140.1, 130.7, 130.3, 130.1, 130.0 (2C), 126.4, 121.6, 119.0, 114.7 (2C), 51.4, 48.0. LCMS: t_{R}=9.63 min. [M+H]⁺=402.3; 404.3. HPLC (λ₂₈₀): Purity 96.0%; HPLC (λ₂₈₀): Purity 98.1%; t_{R}: 11.6 min (method 3).

### MCK349: 1-(3-bromobenzyl)-N³-(3,4-dichlorophenyl)-1H-1,2,4-triazole-3,5-diamine

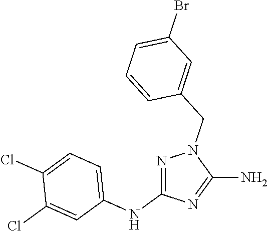 [SMILES: Nc1nc(Nc2ccc(Cl)c(Cl)c2)nn1Cc1cccc(Br)c1]

N³-(3,4-dichlorophenyl)-1H-1,2,4-triazole-3,5-diamine (250 mg, 1.03 mmol) was dissolved in anhydrous DMF (1 mL), then potassium tert-butoxide (115 mg, 1.03 mmol) was added at 0° C. 3-Bromobenzyl bromide (270 mg, 1.08 mmol), dissolved in anhydrous CH₂Cl₂ (1 mL), was added dropwise and the reaction mixture was stirred at 0° C. for 1 h and at r.t. for 16 h. The reaction mixture was quenched with water (1 mL) and the solvents were evaporated under reduced pressure. Purification by reversed-phase chromatography (H₂O/CH₃CN, 10/0 then 45/55) afforded the titled compound as a white solid (93.9 mg, 0.229 mmol, 22% yield). Rf (CH₂Cl₂/THF, 1/1, v/v)=0.39. M.p.=150° C. ¹H NMR (400 MHz, DMSO-d₆) δ 9.18 (s, 1H, NH), 7.82 (d, J=2.3 Hz, 1H, CH_{Ar}), 7.49 (ddd, J=7.9, 2.2, 1.1 Hz, 1H, CH_{Ar}), 7.44 (t, J=1.8 Hz, 1H, CH_{Ar}), 7.40-7.30 (m, 3H, CH_{Ar}), 7.24 (dt, J=7.8, 1.3 Hz, 1H, CH_{Ar}), 6.45 (s, 2H, NH₂), 5.05 (s, 2H, CH₂). ¹³C NMR (101 MHz, DMSO-d₆) δ 156.6, 154.3, 142.3, 140.0, 130.7, 130.7, 130.2, 130.1, 130.0, 126.4, 121.6, 119.4, 116.3, 115.8, 48.0. LCMS: t_{R}=11.64 min. [M+H]⁺=412.3; 414.4. HPLC (λ₂₅₄): Purity 97.7%; HPLC (λ₂₈₀): Purity 96.4%; t_{R}: 15.0 min (method 3).

### MCK350: N³-(3,4-dichlorophenyl)-1-(4-isopropylbenzyl)-1H-1,2,4-triazole-3,5-diamine

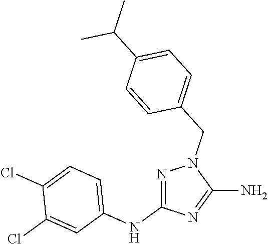 [SMILES: CC(C)c1ccc(Cn2nc(Nc3ccc(Cl)c(Cl)c3)nc2N)cc1]

N³-(3,4-dichlorophenyl)-1H-1,2,4-triazole-3,5-diamine (250 mg, 1.03 mmol) was dissolved in anhydrous DMF (1 mL), then potassium tert-butoxide (115 mg, 1.03 mmol) was added at 0° C. 4-Isopropylbenzyl chloride (0.18 mL, 1.08 mmol), dissolved in anhydrous CH₂Cl₂ (1 mL), was added dropwise and the reaction mixture was stirred at 0° C. for 1 h and at r.t. for 6 h. The reaction mixture was quenched with water (1 mL) and the solvents were evaporated under reduced pressure. Purification by reversed-phase chromatography (H₂O/CH₃CN, 10/0 then 45/55) afforded the titled compound as an orange solid (162 mg, 0.432 mmol, 42% yield) along with the N₅-benzylated regioisomer. Rf (CH₂Cl₂/THF, 1/1, v/v)=0.53. M.p.=138° C. ¹H NMR (400 MHz, DMSO-d₆) δ 9.14 (s, 1H, NH), 7.83 (dd, J=1.8, 1.0 Hz, 1H, CH_{Ar}), 7.45-7.30 (m, 2H, CH_{Ar}), 7.26-7.11 (m, 4H, CH_{Ar}), 6.38 (s, 2H, NH₂), 4.99 (s, 2H, CH₂), 2.84 (hept, J=6.9 Hz, 1H, CH), 1.16 (d, J=6.9 Hz, 6H, 2CH₃). ¹³C NMR (101 MHz, DMSO-d₆) δ 156.3, 154.1, 147.4, 142.4, 134.7, 130.7, 130.2, 127.3 (2C), 126.2 (2C), 119.3, 116.3, 115.8, 48.5, 33.1, 23.8 (2C). LCMS: t_{R}=12.49 min. [M+H]⁺=376.4; 378.4. HPLC (λ₂₅₄): Purity 95.4%; t_{R}: 17.1 min (method 3).

### MCK351: 1-(3-bromobenzyl)-N³-(pyridin-3-yl)-1H-1,2,4-triazole-3,5-diamine

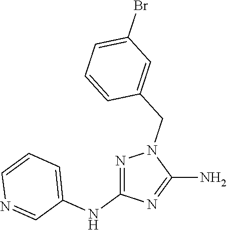 [SMILES: Nc1nc(Nc2cccnc2)nn1Cc1cccc(Br)c1]

N³-(pyridin-3-yl)-1H-1,2,4-triazole-3,5-diamine (250 mg, 1.42 mmol) was dissolved in anhydrous DMF (1 mL), then potassium tert-butoxide (159 mg, 1.42 mmol) was added at 0° C. 3-Bromobenzyl bromide (373 mg, 1.49 mmol), dissolved in anhydrous CH₂Cl₂ (1 mL), was added dropwise and the reaction mixture was stirred at 0° C. for 1 h and at r.t. for 4 h. The reaction mixture was quenched with water (1 mL) and the solvents were evaporated under reduced pressure. Purification by silica gel flash chromatography (CH₂Cl₂/MeOH, 10/0 then 9/1) afforded the titled compound as a brown solid (141.3 mg, 0.411 mmol, 29% yield). Rf (CH₂Cl₂/MeOH, 9/1, v/v)=0.44. M.p.=92-94° C. ¹H NMR (400 MHz, DMSO-d₆) δ 8.98 (s, 1H), 8.63 (dd, J=2.7, 0.7 Hz, 1H), 7.94 (dd, J=4.6, 1.5 Hz, 1H), 7.91 (ddd, J=8.4, 2.7, 1.5 Hz, 1H), 7.50-7.43 (m, 2H), 7.33 (t, J=7.7 Hz, 1H), 7.25 (dt, J=7.8, 1.4 Hz, 1H), 7.22-7.14 (m, 1H), 6.41 (s, 2H), 5.04 (s, 2H). ¹³C NMR (101 MHz, DMSO-d₆) δ 156.8, 154.4, 140.1, 139.5, 138.8, 138.0, 130.6, 130.1, 130.0, 126.4, 123.2, 121.1, 121.4, 48.0. LCMS: t_{R}=6.91 min. [M+H]⁺=345.5; 347.4. HPLC (λ₂₅₄): Purity 96.6%; HPLC (λ₂₈₀): Purity 96.5%; t_{R}: 7.39 min (method 4).

### MCK352:1-(3-bromobenzyl)-N⁴-(pyridin-3-yl)-1H-1,2,4-triazole-3,5-diamine

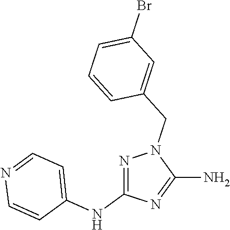 [SMILES: Nc1nc(Nc2ccncc2)nn1Cc1cccc(Br)c1]

N⁴-(pyridin-3-yl)-1H-1,2,4-triazole-3,5-diamine (250 mg, 1.42 mmol) was dissolved in anhydrous DMF (1 mL), then potassium tert-butoxide (159 mg, 1.42 mmol) was added at 0° C. 3-Bromobenzyl bromide (373 mg, 1.49 mmol), dissolved in anhydrous DMF (1 mL), was added dropwise and the reaction mixture was stirred at 0° C. for 1 h and at r.t. for 1 h 30. The reaction mixture was quenched with water (1 mL) and the solvents were evaporated under reduced pressure. Purification by reversed-phase chromatography (H₂O/CH₃CN, 10/0 then 45/55) afforded the titled compound as an orange solid (68.7 mg, 0.200 mmol, 14% yield). Rf (CH₂Cl₂/THF, 1/1, v/v)=0.72. ¹H NMR (400 MHz, DMSO-d₆) δ 9.37 (s, 1H, NH), 8.19 (d, J=6.4 Hz, 2H, CH_{Ar}), 7.49 (ddd, J=7.9, 2.1, 1.2 Hz, 1H, CH_{Ar}), 7.45 (t, J=1.8 Hz, 1H, CH_{Ar}), 7.40-7.36 (m, 2H, CH_{Ar}), 7.33 (t, J=7.8 Hz, 1H, CH_{Ar}), 7.25 (dt, J=7.8, 1.3 Hz, 1H, CH_{Ar}), 6.47 (s, 2H, NH₂), 5.07 (s, 2H, CH₂). ¹³C NMR (101 MHz, DMSO-d₆) δ 156.2, 154.4, 149.5 (2C), 148.0, 140.0, 130.7, 130.2, 130.0, 126.4, 121.6, 110.3 (2C), 48.1. LCMS: t_{R}=5.50 min. [M+H]⁺=345.5; 347.4. HPLC (λ₂₅₄): Purity 97.7%; HPLC (λ₂₈₀): Purity 100%; t_{R}: 7.22 min (method 4).

### Example B: Biology

### I. Material and Methods

### Cell Culture

ARPE-19 (ATCC CRL-2302) and TIME (ATCC CRL-4025) endothelial cells were purchased from the American Tissue Culture Collection. ARPE-19 cells were cultured in DMEM:F12 medium and 10% FBS. TIME cells were cultured in Vascular Cell Basal Medium (ATCC® PCS-100-030) and 6% FBS.

### Cell Viability (XTT Assay)

ARPE-19 cells were incubated in a 96-well plate with different effectors for 48 hours. Fifty microliters of sodium 3′-[1-phenylaminocarbonyl)-3,4-tetrazolium]-bis(4-methoxy-6-nitro) benzene sulfonic acid hydrate (XTT) reagent was added to each well. The test is based on the cleavage of the yellow tetrazolium salt XTT to form an orange formazan dye by metabolically active cells. This bio-reduction occurs in viable cells only and is associated with NAD(P)H production through glycolysis. Therefore, the amount of formazan dye measured at 490 nm, correlates directly with the number of metabolically active cells reflecting cell proliferation, viability, and cytotoxicity. Each assay was performed in quadruplicate.

### ROS Labeling

ARPE-19 cells were treated with MCK349 30 minutes then with 200 μM H2O2 for 2 h. 10 μM DCF-DA (2′,7′-Dichlorofluorescein diacetate) were added on cell media for 30 min. Cells were rinsed then detached (trypsin) and resuspended in PBS 0.5% BSA and 2 mM EDTA. The cells are analyzed by cytometry (ROS labeled with DCFDA-FITC).

Quantitative Real-Time PCR (qPCR) Experiments

One microgram of total RNA was used for the reverse transcription, using the QuantiTect Reverse Transcription kit (QIAGEN, Hilden, Germany), with blend of oligo (dT) and random primers to prime first-strand synthesis. SYBR master mix plus (Eurogentec) was used for qPCR. The mRNA level was normalized to 36B4 mRNA

### Immunoblotting

Cells were lysed in buffer containing 3% SDS, 10% glycerol and 0.825 mM Na₂HPO₄. 30 to 50 μg of proteins separated on 10% SDS-PAGE, transferred onto a PVDF membrane and then exposed to the appropriate antibodies. Proteins were visualized with the ECL system using horseradish peroxidase-conjugated anti-rabbit or anti-mouse antibodies.

### Vessels Formation Assays

300 μL of Matrigel Matrix were coated in 24-well plates and put 1 h a 37° C. Then Time cells were plated (300 000 cells/well). Plates were then incubated at 37° C., 5% CO₂ for 24 hours. Representative images were taken with the Evos XL Core Cell Imaging System at low magnification (4×).

### Endothelial Cells Proliferation

TIME cells were plated in 12-well plates (25 000 cells/well) in 0.5% SVF media and then stimulated with 100 ng/mL CXCL8 or CXCL7 for 72 h. Cell proliferation is assessed by cell count. The results are expressed as % of day 0 relative to the condition tested.

### LPS Stimulated Whole Blood

Human peripheral volunteers were obtained from healthy donors with informed consent following the Declaration of Helsinki according to recommendations of an independent scientific review board. The project has been validated by The Etablissement Français du Sang, the French national agency for blood collection (protocol 7952NQ). Blood samples were collected using ethylene diamine tetraacetic acid-containing tubes.

Whole blood will be removed from a single donor (K3-EDTA) and diluted 1:5 with RPMI 1640. Within 30 minutes of sampling, blood will be incubated with 5 or 10 μM MCK349 for 1 h prior to 10 ng/ml LPS stimulation for 18 h. Blood is centrifuged to recover plasma and cytokines are analyzed by multiplex ELISA (Meso Scale Discovery).

### ARPE-19 Stimulation to Mimic Diabetic Retinopathy In Vitro

ARPE-19 cells were cultured in hyperglycaemic conditions (D-glucose, 25 mmol/l) for 16 days at 37° C. under 5% CO₂ in medium (DMEM/F12) supplemented with 10% FBS and 1% penicillin. On day 16, cells were pre-treated with MCK349. On day 17, cells were washed and treated with MCK349 and cytokines (10 ng/ml IL1ß, 25 ng/ml VEGF and 25 ng/ml TNFα) for 24 h. Cytokines are analyzed by multiplex ELISA (Meso Scale Discovery) on the supernatant. Cells were recovered to analyze the mRNA level of different markers by qPCR.

### Multiplex ELISA (Meso Scale Discovery)

Cytokines from plasma (whole blood) or supernatant (ARPE-19 cells) were analyzed by multiplex ELISA. A multiplex ELISA was designed from U-PLEX Biomarker Group 1 (hu) Assays (K15069L-1). The protocol performed follows the instructions of the supplier.

### Zebrafish Metastatic Tumor Model

All animal experiments were approved by the Experimental Animal Ethical Committee. Transgenic zebrafish embryos (fli: EGFP, blood vessels marked in GFP) were raised at 28° C. under standard experimental conditions. Zebrafish embryos at the age of 24 hpf were incubated in aquarium water containing 0.2 mmol/L 1-phenyl-2-thio-urea (PTU, Sigma). At 48-hpf, zebrafish embryos were placed for 72 h in normoxia (Nx) or hypoxia 5% oxygen (Nx), in the presence of treatment. Retinal neo-angiogenesis was analyzed by fluorescent confocal microscope and quantified by ImageJ.

### Laser Induced Choroidal Neovascularization and Vascular Leakage in Mice

Laser photocoagulation (shoot retina with laser beam (50 μm, 0.1 s, and 250~300 mW or any other parameters reasonable) for 4 spots avoiding blood vessels) were performed binocularly on each animal at Day 1 on male C57 mice (8 mice per group).

Vehicle and MCK349 (52 μg/ml) were delivered by binocular intravitreal injection (2 μl) four times in total. Once on Day 1 (right after modeling), Day 4, Day 7 and Day 11. Optical Coherence Tomography (OCT) was performed once on modeling day right after laser photocoagulation and once on Day 9 and Day 15. CNV volume of each lesion will be measured. Fundus Fluorescein Angiography (FFA) was conducted once on Day 9 and Day 15. The angiograms will be graded as follows: Grade 0, no bright hyperfluorescence; Grade 1, hyperfluorescence without leakage; Grade 2a, hyperfluorescence and late leakage; and Grade 2b, bright hyperfluorescence and late leakage beyond treated areas.

### Statistical Analysis

All data are expressed as the mean±the standard error (SD). Statistical significance and p values were determined with Prism 5.0b (GraphPad Software) by a one-way ANOVA with Bonferroni post hoc or with the two-tailed Student's 1-test.

### Screening of MCK Compound (Table 1 Below)

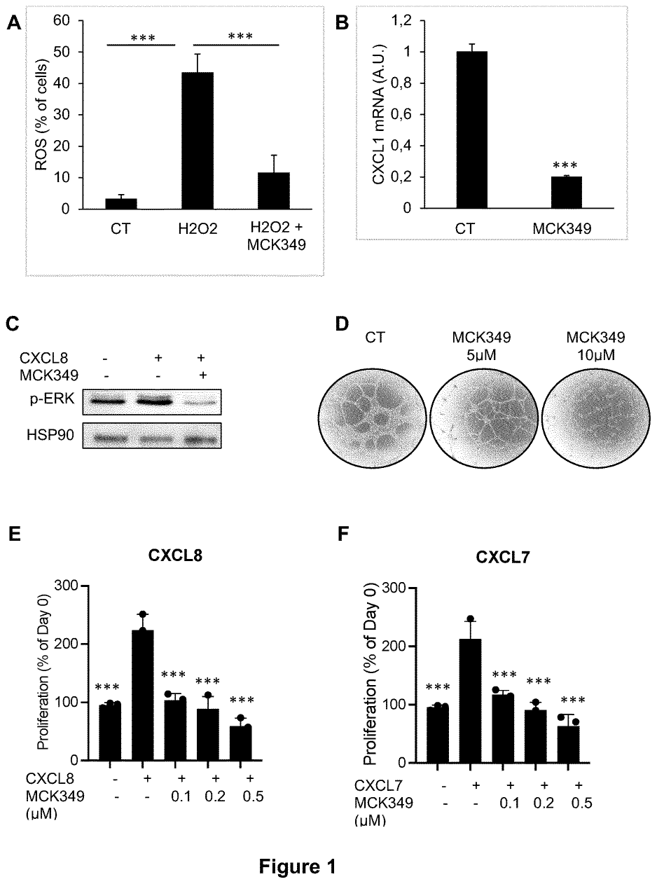

All compounds were evaluated in different experiments. Results are represented as the mean of three independent experiments. A. ARPE-19 cells were treated with dose response of compound for 48 h. Values are reported as IC50 measured with the XTT assay. B and C. ARPE-19 cells were treated with 5 μM (B) or 1 μM (C) compound then with 200 μM H₂O₂ for 2 h. ROS were analyzed by cytometry. A score of 1 means that the compound inhibited ROS by at least 50% (significant inhibition). A score of 0 means that the compound inhibited ROS by less than 50% (non-significant inhibition). As illustrated in FIG. 1A. D. ARPE-19 cells were treated with 2.5 μM compound for 48 h and the level of CXCL1 mRNA was evaluated by qPCR. A score of 1 means that the compound inhibited CXCL1 mRNA by at least 50% (significant inhibition). A score of 0 means that the compound inhibited CXCL1 mRNA by less than 50% (non-significant inhibition). As illustrated in FIG. 1B. E. TIME cells were pre-treated with 2.5 μM compound for 1 h then stimulated with 100 ng/ml CXCL8 for 15 min p-ERK levels were analyzed by immunoblotting. HSP90 served as loading control. A score of 1 means that the compound inhibited ERK activation (p-ERK) by at least 50% (significant inhibition). A score of 0 means that the compound inhibited ERK activation (p-ERK) by less than 50% (non-significant inhibition). As illustrated in FIG. 1C.

### II. Results

TABLE 1

| | | B. ROS | C. ROS | D. CXCL1 | | |
|---|---|---|---|---|---|---|
| | A. Safety | inhibition | inhibition | mRNA | | |
| | IC50 on | 5 μM - | 1 μM - | inhibition | E. p-ERK | |
| MCK | ARPE-19 | ARPE-19 | ARPE-19 | ARPE-19 | inhibition - | Total |
| Compounds | cells | cells | cells | cells | TIME | score |
| 248 | >25 μM | 1 | 0 | 0 | 0 | 1 |
| 249 | >25 μM | 1 | 0 | 0 | 0 | 1 |
| 250 | >25 μM | 1 | 1 | 0 | 0 | 2 |
| 253 | >25 μM | 0 | 0 | 1 | 0 | 1 |
| 254 | >25 μM | 0 | 0 | 1 | 0 | 1 |
| 257 | >25 μM | 1 | 1 | 1 | 1 | 4 |
| 259 | >25 μM | 0 | 0 | 1 | 1 | 2 |
| 260 | >25 μM | 1 | 1 | 0 | 1 | 3 |
| 261 | >25 μM | 1 | 0 | 0 | 1 | 2 |
| 262 | >25 μM | 1 | 1 | 0 | 1 | 3 |
| 263 | >25 μM | 1 | 1 | 1 | 1 | 4 |
| 264 | >25 μM | 1 | 1 | 1 | 1 | 4 |
| 268 | >25 μM | 1 | 0 | 0 | 1 | 2 |
| 269 | >25 μM | 1 | 1 | 0 | 1 | 3 |
| 271 | >25 μM | 1 | 1 | 0 | 1 | 3 |
| 272 | >25 μM | 1 | 0 | 1 | 1 | 3 |
| 273 | >25 μM | 1 | 1 | 0 | 1 | 3 |
| 274 | >25 μM | 1 | 1 | 0 | 0 | 2 |
| 275 | >25 μM | 1 | 1 | 0 | 0 | 2 |
| 276 | >25 μM | 0 | 0 | 0 | 1 | 1 |
| 278 | >25 μM | 0 | 0 | 0 | 1 | 1 |
| 279 | >25 μM | 0 | 0 | 0 | 1 | 1 |
| 280 | >25 μM | 0 | 0 | 0 | 1 | 1 |
| 281 | >25 μM | 0 | 0 | 0 | 1 | 1 |
| 282 | >25 μM | 0 | 0 | 1 | 1 | 2 |
| 283 | >25 μM | 0 | 0 | 1 | 1 | 2 |
| 284 | >25 μM | 0 | 0 | 0 | 1 | 1 |
| 288 | >25 μM | 1 | 1 | 0 | 0 | 2 |
| 289 | >25 μM | 1 | 1 | 0 | 0 | 2 |
| 290 | >25 μM | 1 | 1 | 0 | 1 | 3 |
| 292 | >25 μM | 0 | 0 | 0 | 1 | 1 |
| 295 | >25 μM | 0 | 0 | 0 | 1 | 1 |
| 297 | >25 μM | 0 | 0 | 0 | 1 | 1 |
| 298 | >25 μM | 0 | 0 | 1 | 1 | 2 |
| 299 | >25 μM | 0 | 0 | 0 | 1 | 1 |
| 300 | >25 μM | 0 | 0 | 0 | 1 | 1 |
| 301 | >25 μM | 0 | 0 | 1 | 0 | 1 |
| 338 | >25 μM | 1 | 1 | 1 | 0 | 3 |
| 339 | >25 μM | 1 | 0 | 1 | 0 | 2 |
| 340 | >25 μM | 0 | 0 | 1 | 0 | 1 |
| 341 | >25 μM | 1 | 1 | 1 | 0 | 3 |
| 342 | >25 μM | 1 | 1 | 1 | 0 | 3 |
| 343 | >25 μM | 1 | 0 | 1 | 1 | 3 |
| 344 | >25 μM | 1 | 1 | 1 | 1 | 4 |
| 345 | >25 μM | 1 | 1 | 1 | 1 | 4 |
| 346 | >25 μM | 1 | 1 | 0 | 1 | 3 |
| 347 | >25 μM | 0 | 0 | 1 | 1 | 2 |
| 348 | >25 μM | 1 | 1 | 1 | 1 | 4 |
| 349 | >25 μM | 1 | 1 | 1 | 1 | 4 |
| 350 | >25 μM | 1 | 1 | 1 | 1 | 4 |
| 351 | >25 μM | 0 | 0 | 1 | 1 | 2 |
| 352 | >25 μM | 1 | 0 | 1 | 1 | 3 |

The inventors evaluated the compounds via a screen for their ability to:
i) be non-toxic on retinal cells (IC50>25 μM), Table 1. A),
ii) inhibit reactive oxygen species (ROS, Table 1. B-C and FIG. 1A),
iii) inhibit CXCR2 and its signaling: decrease of CXCL1 transcription (Table 1. D and FIG. 1B) and inhibition of ERK (p-ERK) activation after stimulation by CXCL8 (Table 1. E and FIG. 1C).

The inventors also demonstrated, that the compound MCK349 is able to inhibit angiogenesis in vitro by inhibiting vessel formation on matrigel (FIG. 1D) and by inhibiting endothelial cell proliferation induced by CXCL8 (FIG. 1E) and CXCL7 (FIG. 1F).

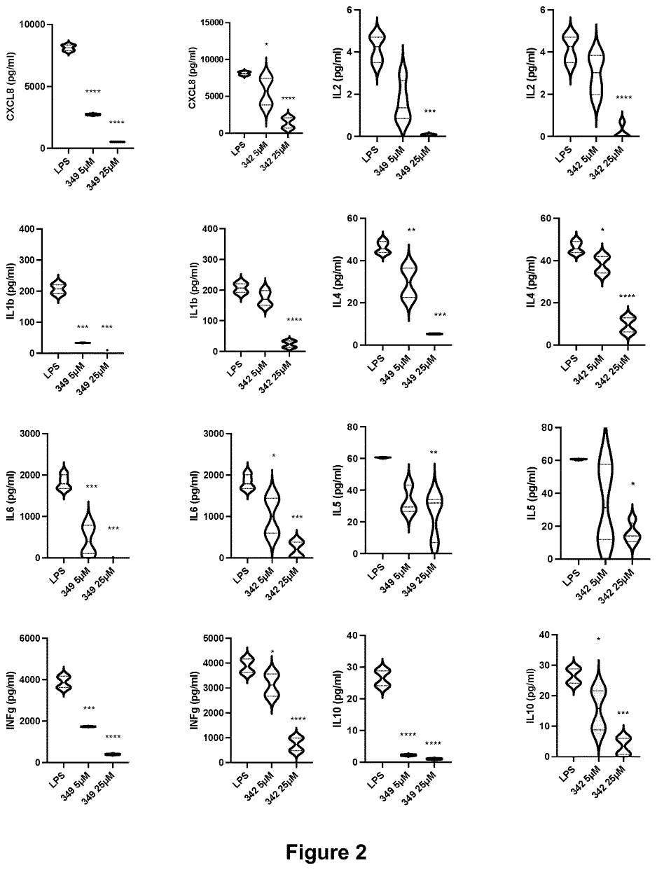

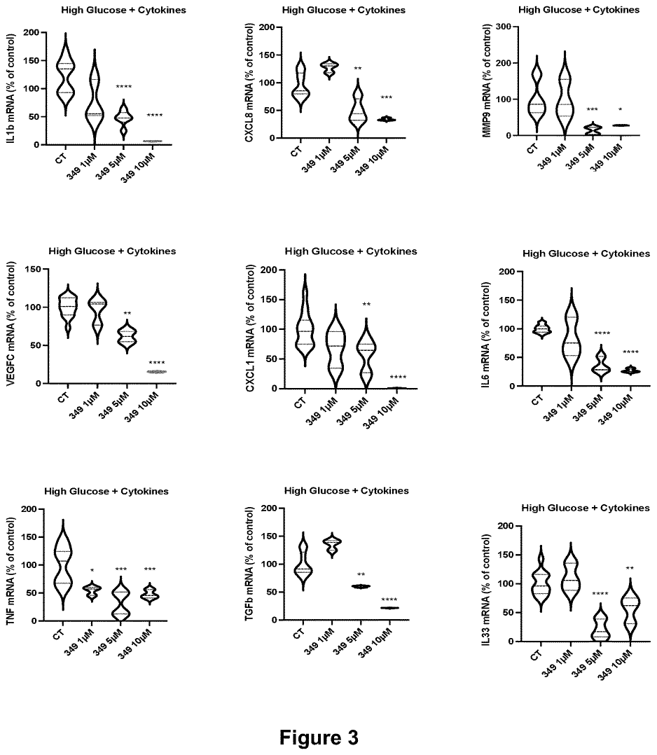

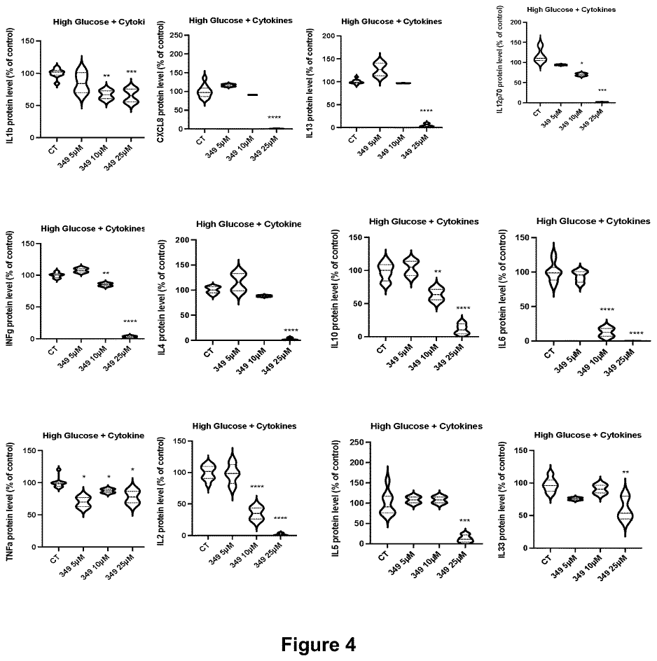

They also demonstrated that MCK342 and MCK349 were able to inhibit inflammation. More particularly, they have shown that MCK342 and MCK349 were able to inhibit pro-inflammatory cytokines (CXCL8, IL2, IL1b, IL4, IL6, IL5, INFg, IL10) induced by LPS stimulation in whole blood (FIG. 2) and inhibit the expression (mRNA, FIG. 3) and secretion (FIG. 4) of pro-angiogenic and/or pro-inflammatory cytokines (IL1b, CXCL8, MMP9, VEGFC, CXCL1, IL6, TNFa, TGFb, IL33, IL13, IL12p70, INFg, IL4, IL10, IL6, IL5, IL33) in diabetic retinopathy model in vitro.

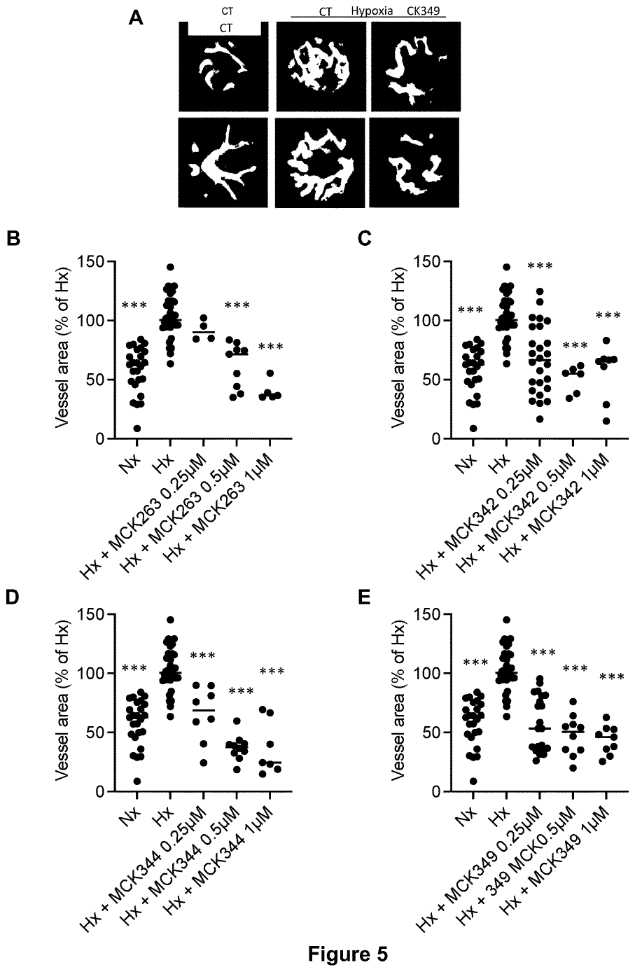

The inventors have further developed a model of retinal neoangiogenesis in zebrafish (fluorescent blood vessels, in white in the FIG. 5A) induced by hypoxia. They demonstrated that the compounds MCK263, MCK342, MCK344, and MCK349 were able to inhibit ocular neoangiogenesis in this in vivo model (FIG. 5).

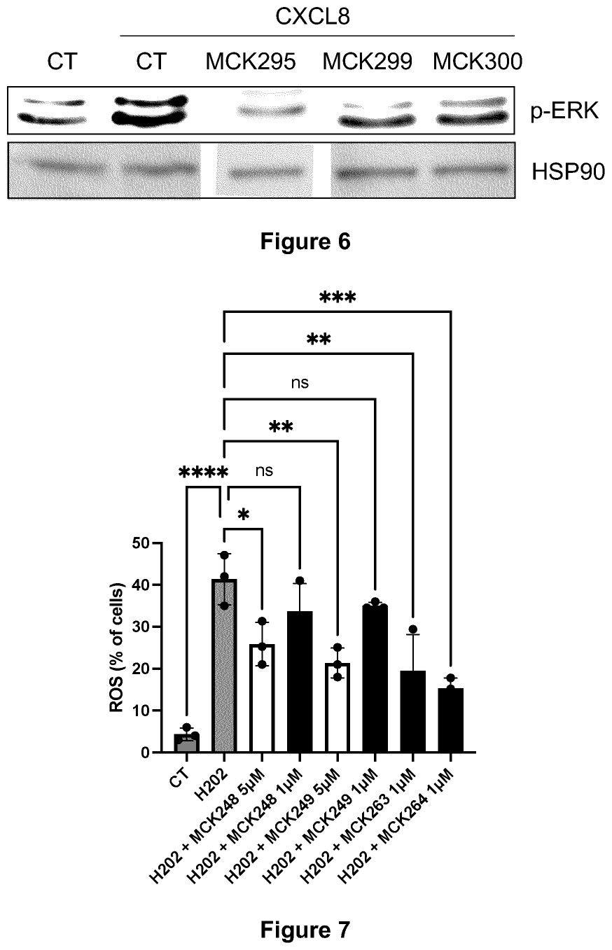

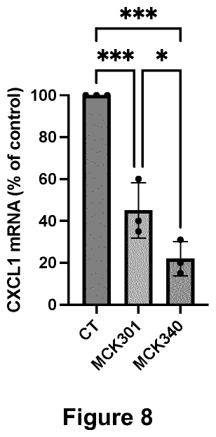

They also showed that substituted compounds comprising at least one substituent R₂ as defined herein exhibit an improved efficacy compared to unsubstituted compounds (n=0), for inhibiting the activation of p-ERK induced by CXCL8 (FIG. 6), for inhibiting of ROS (FIG. 7), and for inhibiting the expression of CXCL1 (FIG. 8).

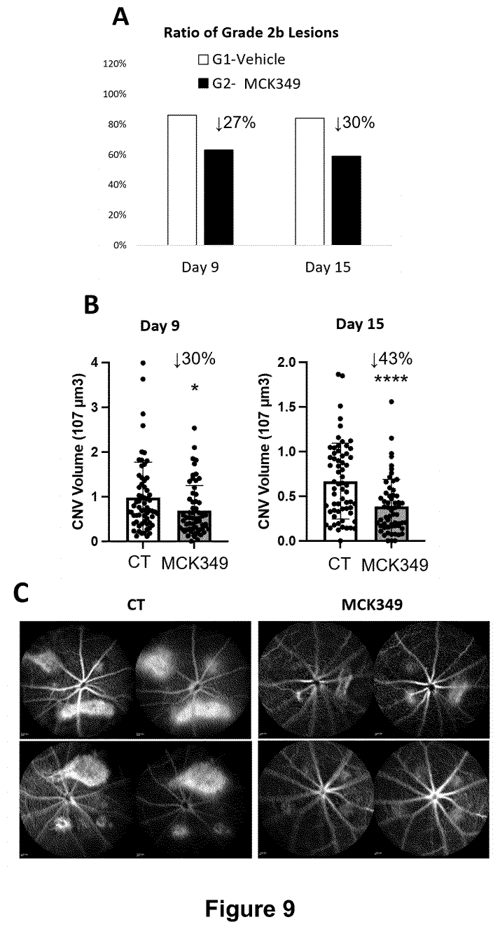

In addition, in a laser-induced Choroidal neovascularization (CNV) model in mice, MCK349 (intravitreal treatment) inhibits vascular leakage (FIGS. 9A and B) and neovascularization (FIG. 9C).

1-16. (canceled)

17. A compound of formula (I): 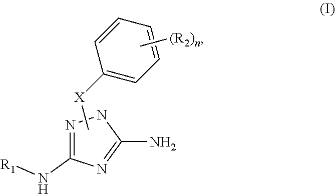
wherein:
X is —(CH₂)—, —(CH₂)—CO—, or —(CH₂)—(CH₂)—;
R₁ is an aryl or a heteroaryl group, said aryl or heteroaryl groups are optionally substituted by at least one radical selected from the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro;
n is 0, 1, 2, 3, 4, or 5;
each R₂ is independently a radical selected from the group consisting of a (C₁-C₆)alkyl optionally substituted by at least one halogen, a (C₁-C₆)alkyloxy optionally substituted by at least one halogen, a halogen, a —CO₂R₃ with R₃ being a hydrogen or a (C₁-C₆)alkyl, and a —C(O)R₄ with R₄ being a hydrogen, a (C₁-C₆)alkyl, or a —NR₅R₆ with R₅ and R₆ are independently a hydrogen or a (C₁-C₆)alkyl, a nitrile, and a nitro;
with the proviso that said compound is not a compound selected from the group consisting of:
1-benzyl-N³-phenyl-1H-1,2,4-triazole-3,5-diamine; and
1-phenethyl-N³-phenyl-1H-1,2,4-triazole-3,5-diamine
or a pharmaceutically acceptable salt thereof.

18. The compound according to claim 17, wherein n is 1, 2, 3, 4, or 5.

19. The compound according to claim 17, wherein said compound is of the formula (IA):

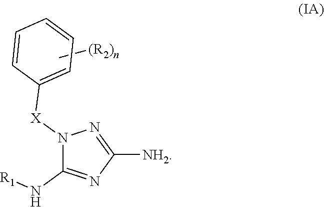

20. The compound according to claim 17, wherein said compound is of the formula (IB):

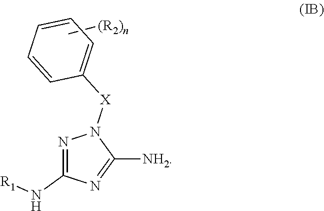

21. The compound according to claim 17, wherein X is —(CH₂)—.

22. The compound according to claim 17, wherein R₁ is a phenyl or a pyridinyl group, said phenyl or pyridinyl groups are optionally substituted by at least one radical selected from the group consisting of a (C₁-C₆)alkyloxy, a (C₁-C₆)alkyl, a halogen and a —CO₂R₃ with R₃ being a (C₁-C₆)alkyl.

23. The compound according to claim 17, wherein n is 1, 2, or 3.

24. The compound according to claim 17, wherein each R₂ is independently a radical selected from the group consisting of a (C₁-C₆)alkyl optionally substituted by at least one halogen, a (C₁-C₆)alkyloxy, a halogen, a nitro, and a nitrile.

25. The compound according to claim 24, wherein each R₂ is independently a radical selected from the group consisting of a methyl, a trifluoromethyl, an isopropyl, a methoxy, a fluorine, a bromine, a chlorine, a nitro, and a nitrile.

26. The compound according to claim 17, wherein each R₂ is independently a radical selected from the group consisting of an isopropyl and a bromine.

27. The compound according to claim 17, wherein:
X is —(CH₂)—;
R₁ is a phenyl or a pyridinyl group, said phenyl or pyridinyl groups are optionally substituted by at least one radical selected from the group consisting of a methyl, a chlorine, a bromine, and a —CO₂R₃ with R₃ being a methyl;
n is 1; and
R₂ is a radical selected from the group consisting of an isopropyl and a bromine.

28. The compound according to claim 17, wherein said compound is selected from the group consisting of:
MCK248: 1-benzyl-N⁵-phenyl-1H-1,2,4-triazole-3,5-diamine;
MCK250: 1-(2-methylbenzyl)-N³-phenyl-1H-1,2,4-triazole-3,5-diamine;
MCK253: 1-benzyl-N³-(pyridin-2-yl)-1H-1,2,4-triazole-3,5-diamine;
MCK254: 1-benzyl-N³-(pyridin-3-yl)-1H-1,2,4-triazole-3,5-diamine;
MCK257: 1-(2,6-difluorobenzyl)-N⁵-phenyl-1H-1,2,4-triazole-3,5-diamine;
MCK259: 1-(2,6-difluorobenzyl)-N³-(pyridin-2-yl)-1H-1,2,4-triazole-3,5-diamine;
MCK260: 1-(3-methoxybenzyl)-N⁵-phenyl-1H-1,2,4-triazole-3,5-diamine;
MCK261: 1-(3-methoxybenzyl)-N³-phenyl-1H-1,2,4-triazole-3,5-diamine;
MCK262: 1-(4-isopropylbenzyl)-N⁵-phenyl-1H-1,2,4-triazole-3,5-diamine;
MCK263: 1-(4-isopropylbenzyl)-N³-phenyl-1H-1,2,4-triazole-3,5-diamine;
MCK264: 1-(3-bromobenzyl)-N³-phenyl-1H-1,2,4-triazole-3,5-diamine;
MCK268: 1-(2,6-dichlorobenzyl)-N³-phenyl-1H-1,2,4-triazole-3,5-diamine;
MCK269: 1-(2-methylbenzyl)-N³-(pyridin-2-yl)-1H-1,2,4-triazole-3,5-diamine;
MCK271: 1-(4-methylbenzyl)-N³-phenyl-1H-1,2,4-triazole-3,5-diamine;
MCK272: 1-(4-chlorobenzyl)-N³-phenyl-1H-1,2,4-triazole-3,5-diamine;
MCK273: N³-phenyl-1-(3-(trifluoromethyl)benzyl)-1H-1,2,4-triazole-3,5-diamine;
MCK274: 1-(3,5-difluorobenzyl)-N³-phenyl-1H-1,2,4-triazole-3,5-diamine;
MCK275: 1-(3-nitrobenzyl))-N³-phenyl-1H-1,2,4-triazole-3,5-diamine;
MCK276: 1-(3-bromobenzyl)-N³-(pyridin-2-yl)-1H-1,2,4-triazole-3,5-diamine;
MCK278: 1-(2,6-dichlorobenzyl)-N³-(pyridin-2-yl)-1H-1,2,4-triazole-3,5-diamine;
MCK279: 1-(4-methylbenzyl)-N³-(pyridin-2-yl)-1H-1,2,4-triazole-3,5-diamine;
MCK280: 1-(4-chlorobenzyl)-N³-(pyridin-2-yl)-1H-1,2,4-triazole-3,5-diamine;
MCK281: 4-((5-amino-3-(pyridin-2-ylamino)-1H-1,2,4-triazol-1-yl)methyl)benzonitrile;
MCK282: N³-(pyridin-2-yl)-1-(3-(trifluoromethyl)benzyl)-1H-1,2,4-triazole-3,5-diamine;
MCK283: 1-(3,5-difluorobenzyl)-N³-(pyridin-2-yl)-1H-1,2,4-triazole-3,5-diamine;
MCK284: 1-(2,4-difluorobenzyl)-N³-(pyridin-2-yl)-1H-1,2,4-triazole-3,5-diamine;
MCK288: 1-(4-nitrobenzyl))-N³-phenyl-1H-1,2,4-triazole-3,5-diamine;
MCK289: 1-(2-nitrobenzyl)-N³-(pyridin-2-yl)-1H-1,2,4-triazole-3,5-diamine;
MCK290: 1-(4-nitrobenzyl)-N³-(pyridin-2-yl)-1H-1,2,4-triazole-3,5-diamine;
MCK292: 1-(3-nitrobenzyl)-N³-(pyridin-2-yl)-1H-1,2,4-triazole-3,5-diamine;
MCK295: 2-(5-amino-3-(pyridin-2-ylamino)-1H-1,2,4-triazol-1-yl)-1-(3-nitrophenyl)ethan-1-one;
MCK297: 1-(2-nitrobenzyl)-N³-phenyl-1H-1,2,4-triazole-3,5-diamine;
MCK298: N³-phenyl-1-(3,4,5-trimethoxybenzyl)-1H-1,2,4-triazole-3,5-diamine;
MCK299: 2-(5-amino-3-(phenylamino)-1H-1,2,4-triazol-1-yl)-1-phenylethan-1-one;
MCK300: 1-phenethyl-N⁵-phenyl-1H-1,2,4-triazole-3,5-diamine;
MCK338: N⁵-phenyl-1-(3-(trifluoromethyl)benzyl)-1H-1,2,4-triazole-3,5-diamine;
MCK339: 1-(3-bromobenzyl)-N⁵-phenyl-1H-1,2,4-triazole-3,5-diamine;
MCK340: 1-(3-bromobenzyl)-N³-(4-methoxyphenyl)-1H-1,2,4-triazole-3,5-diamine;
MCK341: 1-(3-bromobenzyl)-N⁵-(4-methylphenyl)-1H-1,2,4-triazole-3,5-diamine;
MCK342: 1-(3-bromobenzyl)-N³-(4-methylphenyl)-1H-1,2,4-triazole-3,5-diamine;
MCK343: 1-(3-bromobenzyl)-N⁵-(4-chlorophenyl)-1H-1,2,4-triazole-3,5-diamine;
MCK344: 1-(3-bromobenzyl)-N³-(4-chlorophenyl)-1H-1,2,4-triazole-3,5-diamine;
MCK345: 1-(3-bromobenzyl)-N⁵-(4-bromophenyl)-1H-1,2,4-triazole-3,5-diamine;
MCK346: 1-(3-bromobenzyl)-N³-(4-bromophenyl)-1H-1,2,4-triazole-3,5-diamine;
MCK347: methyl-4-((3-amino-1-(3-bromobenzyl)-1H-1,2,4-triazol-5-yl)amino)benzoate;
MCK348: methyl-4-((5-amino-1-(3-bromobenzyl)-1H-1,2,4-triazol-3-yl)amino)benzoate;
MCK349: 1-(3-bromobenzyl)-N³-(3,4-dichlorophenyl)-1H-1,2,4-triazole-3,5-diamine;
MCK350: N³-(3,4-dichlorophenyl)-1-(4-isopropylbenzyl)-1H-1,2,4-triazole-3,5-diamine;
MCK351: 1-(3-bromobenzyl)-N³-(pyridin-3-yl)-1H-1,2,4-triazole-3,5-diamine; and
MCK352: 1-(3-bromobenzyl)-N-(pyridin-3-yl)-1H-1,2,4-triazole-3,5-diamine.

29. A pharmaceutical or veterinary composition comprising a compound according to claim 17 and a pharmaceutically acceptable excipient.

30. A method of treating an ocular disease comprising administering a compound according to claim 17 or a pharmaceutical or veterinary composition thereof to a subject having an ocular disease.

31. The method according to claim 30, wherein the ocular disease is corneal graft angiogenesis, neovascular glaucoma, diabetic retinopathy, corneal diseases induced by new blood vessels, macular degeneration, pterygium, retinal degeneration, retrolental fibroplasia, granular conjunctivitis, macular edema, or Stargardt's disease.

32. The method according to claim 30, wherein said compound or composition is administered by an orally, parenterally, or topically route.

33. The method according to claim 32, wherein said compound or composition is administered by an ocular topical route.

34. The method according to claim 32, wherein the ocular disease is diabetic retinopathy, macular degeneration, neovascular glaucoma, macular edema, or Stargardt's disease.
